(19) United States
(12) Patent Application Publication
(10) Pub. No.: US 2024/0270817 A1
(43) Pub. Date: Aug. 15, 2024

(54) THERAPEUTIC MOLECULES

(71) Applicant: PetMedix Ltd, Cambridge (GB)

(72) Inventor: Marco BARDELLI, London (GB)

(21) Appl. No.: 18/187,414

(22) Filed: Mar. 21, 2023

(30) Foreign Application Priority Data
Feb. 9, 2023 (GB) 2301838.5
Feb. 9, 2023 (GB) 2301839.3

(51) Int. Cl. C07K 14/705; A61P 19/02; C12N 15/63

(52) U.S. Cl. CPC C07K 14/70578; A61P 19/02; C12N 15/63; C07K 2319/30

(57) ABSTRACT

The present invention relates to effective pain therapies. The invention provides an isolated polypeptide comprising a variant p75 neurotrophin receptor (p75NTR) extracellular domain or a fusion protein containing the same or portions thereof. Nucleic acids encoding the proteins are also encompassed in the invention as well as methods of using the same.

### RELATED APPLICATIONS AND INCORPORATION BY REFERENCE

This application claims the benefit of priority from GB Application No. 2301838.5 and GB Application No. 2301839.3, both applications filed Feb. 9, 2023.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing, which has been submitted electronically and is hereby incorporated by reference in its entirety. Said XML copy, was created Mar. 21, 2023, is named Y9736_00006.xml and is 187,854 bytes in size.

### FIELD OF THE INVENTION

The present invention relates to effective pain therapies in humans and companion animals.

### BACKGROUND OF THE INVENTION

There is a huge need for therapies for pain relief. Moreover, there is a need for effective pain treatments with minimal side effects.

Pain relief treatments currently include nonsteroidal anti-inflammatory drugs (NSAIDs), and with several nonsteroidal anti-inflammatory drugs which help to control pain and inflammation associated with osteoarthritis. Several NSAIDs have been approved by the FDA. However, there is a need for further effective pain treatments with minimal side effects.

Neurotrophins are a family of proteins involved in proliferation, maintenance, and survival of neurons. Since nerve growth factor (NGF) was discovered in 1950s, numerous biological processes involving NGF have been identified. NGF is critical for proliferation and maintenance of neurons and also plays a role in inflammation and maintenance of pancreatic beta cells. NGF binds to least two receptors, tropomyosin receptor kinase A (TrkA) and low-affinity NGF receptor (LNGFR/p75NTR). The neurotrophin family also includes structurally related brain-derived neurotrophic factor (BDNF), neurotrophin-3 (NT-3), and neurotrophin-4 (NT-4) (also known as NT-5 or NT-4/5). BDNF and NT-4 function primarily through tropomyosin receptor kinase B (TrkB). NT-3 binds with high affinity to tropomyosin receptor kinase C (TrkC) but also is capable of signaling through TrkB. All four neurotrophins bind with low affinity to p75NTR.

NGF causes peripheral sensitization both in vitro and in vivo, as illustrated by the increased response of DRG neurons to temperature or capsaicin in its presence. NGF also leads to transcriptional regulation after retrograde axonal transport, as illustrated by immunostaining showing upregulation of BDNF after intrathecal NGF treatment. Furthermore, NGF can cause sprouting of peripheral afferents into diseased joints and cancerous tissue (Denk et al, Annual Review of Neuroscience, Vol. 40:307-325, 2017).

NGF is expressed at low levels in adulthood, but injury, inflammation or release of NGF cause activation of inflammatory cells. These cells in turn produce and secrete NGF as well and this leads to short-term and long-term effects. NGF has a well-known and multifunctional role in nociceptive processing, although the precise signaling pathways downstream of NGF receptor activation that mediate nociception are complex and not completely understood. The role of NGF in nociception and the generation and/or maintenance of chronic pain has led to it becoming an attractive target of pain therapeutics for the treatment of chronic pain conditions (Barker et al, Journal of Pain Research, 2020:13 1223-1241).

Very low doses of monoclonal antibodies (mAbs) directed against NGF can reduce chronic pain. However, during clinical trials in humans, a small subset of patients treated with mAbs directed against NGF developed rapidly progressive joint degeneration due to interaction with NSAIDs treatment. Complete NGF removal is shown to cause impaired bone and cartilage repairing (Denk et al, Annual Review of Neuroscience, Vol. 40:307-325, 2017). Treatments for use in dogs and cats based on species-specific mAbs that target NGF are now being developed for the management of osteoarthritis (OA)-associated pain (Enomoto et al, Vet Rec. 2019 Jan. 5; 184(1):23 and WO2019177690). However, given the side effects that occurred in clinical trials in humans, there is a need to develop alternative treatments that target NGF.

Citation or identification of any document in this application is not an admission that such document is available as prior art to the present invention.

### SUMMARY OF THE INVENTION

There is a need for effective pain therapies in humans and companion animals. The invention provides a pain therapy with reduced side effects compared to anti-NGF antibody therapies. The proteins of the invention bind NGF which is at elevated levels in pain conditions, thus binding to excess NGF to restore normal NGF levels without completely blocking NGF signalling. Without wishing to be bound by theory, the inventors believe that this ensures a level of NGF signalling which is required for healthy functions. Furthermore, it is believed that the fusion proteins of the invention can operate at a very low dose, but are highly efficacious.

The inventors have used an analgesic strategy to reduce, but not completely deplete, NGF in circulation. To this end, the Extracellular Domain (ECD) of p75 neurotrophin receptor (p75NTR) was used, fused to Fc to increase its half-life. p75NTR binds NGF and other brain-derived neurotrophic factors (BDNF, NT3, NT4) and mediates different cellular activities. The inventors have further used a structure guided mutation strategy to generate p75NTR variants with advantageous physiochemical properties. Point mutations have been introduced into the extracellular domain of p75NTR and used to modulate the binding affinity of p75NTR to its ligand NGF. These variant p75NTR molecules have altered binding characteristics compared to wild-type p75NTR which may allow efficient binding of NGF without completely removing NGF which has been shown to lead to negative side effects such as joint deterioration.

In a first aspect the invention relates to an isolated polypeptide comprising a p75 neurotrophin receptor (p75NTR) extracellular domain, wherein said p75NTR extracellular domain comprises a variant amino acid at one or more of position 75, 109, 133 and/or 134.

The invention also relates to an isolated nucleic acid encoding the isolated polypeptide comprising a p75NTR extracellular domain, wherein said p75NTR extracellular domain comprises a variant amino acid at one or more of position 75, 109, 133 and/or 134.

The invention also relates to a vector comprising a nucleic acid described above.

The invention also relates to a host cell comprising a nucleic acid described above or a vector as described above.

An aspect of the invention relates to a fusion protein comprising a p75NTR extracellular domain, wherein said p75NTR extracellular domain comprises a variant amino acid at one or more of position 75, 109, 133 and/or 134, and a half-life extending moiety.

The invention also relates to nucleic acid encoding a fusion protein as described above.

The invention also relates to a vector comprising a nucleic acid encoding a fusion protein as described above.

The invention also relates to a host cell comprising a nucleic acid encoding a fusion protein as described above, or a vector comprising a nucleic acid encoding a fusion protein as described above.

An aspect of the invention relates to a pharmaceutical composition comprising an isolated polypeptide as described above and herein or a fusion protein as described above and herein.

An aspect of the invention relates to a method for treating an NGF-related disorder in a subject comprising administering an isolated p75NTR protein as described above and herein, a fusion protein as described above and herein, or a pharmaceutical composition as described above and herein.

An aspect of the invention relates to the use of an isolated p75NTR protein as described above and herein, a fusion protein as described above and herein, or a pharmaceutical composition as described above and herein in the treatment of an NGF-related disorder in a subject.

An aspect of the invention relates to an isolated p75NTR protein as described above and herein, or a fusion protein as described above and herein for use in the treatment of an NGF-related disorder in a subject.

An aspect of the invention relates to a method of inhibiting NGF activity in a subject comprising administering an isolated p75NTR protein as described above and herein, a fusion protein as described above and herein, or a pharmaceutical composition as described above and herein.

An aspect of the invention relates to a kit comprising an isolated p75NTR protein as described above and herein, a fusion protein as described above and herein, or a pharmaceutical composition as described above and herein and optionally instructions for use.

In certain embodiments, the p75NTR is from a human. In certain embodiments, the p75NTR is from a non-human primate. In certain embodiments, the p75NTR is from an animal. In certain embodiments, the p75NTR is from a companion animal, including but not limited to a dog, a cat, a horse, a cow, a sheep, or a camel.

In certain embodiments, an isolated p75NTR protein of the invention is used to treat or to inhibit NGF activity in a human. In certain embodiments, an isolated p75NTR protein of the invention is used to treat or to inhibit NGF activity in a non-human primate. In certain embodiments, an isolated p75NTR protein of the invention is used to treat or to inhibit NGF activity in an animal. In certain embodiments, an isolated p75NTR protein of the invention is used to treat or to inhibit NGF activity in a companion animal.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises,” “comprised,” “comprising” and the like can have the meaning attributed to it in U.S. Patent law; e.g., they can mean “includes,” “included,” “including,” and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The invention is described in the following non-limiting figures.

FIG. 1. Sequence alignment of human (SEQ ID NO:73), canine (SEQ ID NO:7), feline (SEQ ID NO:38), equine (SEQ ID NO:5), camel (SEQ ID NO:101), bovine (SEQ ID NO:36), and porcine (SEQ ID NO:110) p75NTR extracellular domains. Amino acids 1-160 are shown. The stalk region and alpha and gamma secretase cleavage sites are not depicted. Sequence alignment performed using EXPASY Clustal Omega. Mutated residues according to the invention are indicated by boxes.

FIG. 2. 3D model of canine p75NTR-ECD (shown as cartoon with transparent surface) in complex with canine NGF (Shown as cartoon) based generated using homology modelling to human pdb 1SG1. Circled are residues of p75NTR-ECD important for binding to NGF.

FIG. 3A-3F. Binding affinity of p75NTR variants. Each panel represent a representative SPR sensogram (showing both raw data as dots and fitted data using 1:1 Langmuir fitting as lines) including 5 different concentrations, of PetML119wt, variants and Bedinvetmab. Table with kinetics values obtained from fitting are also shown.

FIG. 4. Thermal stability analysis of p75NTR variants. Intrinsic fluorescence measurements obtained from each molecules are shown in the graph. Inflection points, highlighted with vertical bars coloured differently, indicate calculated Tm for these molecules.

FIG. 5A-5B. TF-1 cell proliferation assay. In Y-axis Mean Fluorescence Intensity values normalised to controls are shown while in X-axis are shown concentration of either PetML119wt, variants or Bedinvetmab used. Fitted curved using a sigmoidal function are shown as well as IC50 and ICMAX values derived from this for each molecule tested.

FIG. 6A-6B. TrkA activation assay. In Y-axis Luminescence AU values normalised to controls are shown while in X-axis are shown concentration of either PetML119wt, variants or Bedinvetmab used. Fitted curved using a sigmoidal function are shown as well as IC50 and ICMAX values derived from this for each molecule tested.

FIG. 7. Schematic of in vivo study.

FIG. 8A-8C. Human and rat Nerve Growth Factor (h-rNGF) binding affinity determination. Each panel represent a representative SPR sensogram (showing both raw data as dots and fitted data using 1:1 Langmuir fitting as lines) including 5 different concentrations, of PetML308, PetML309 and PetML319. Table with kinetics values obtained from fitting are also shown.

FIG. 9. Thermal denaturation by UnCle. Intrinsic fluorescence measurements obtained from each molecules are shown in the graph for each tested molecules. A table with melting temperatures for each molecules is shown.

FIG. 10. TrkA activation assay. In Y-axis Luminescence AU values normalised to controls are shown while in X-axis are shown concentration of either PetML308, PetML309 and PetML319 used. Fitted curved using a sigmoidal function as well as IC50 and ICMAX values derived from this for each molecule tested.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The invention provides biological therapeutics for human and veterinary use, including p75NTR fusion proteins for use in the treatment of human or companion animals such as dogs, cats, bovines, horses or camels.

### P75NTR Variants

In a first aspect, the invention relates to isolated polypeptide comprising a p75 neurotrophin receptor (p75NTR) extracellular domain, comprising a variant amino acid at one or more of position 75, 109, 133 and/or 134 within said p75NTR.

As used herein, the term p75NTR protein refers to a p75NTR protein that binds NGF and/or other neurotrophins (BDNF, NT-3 and/or NT-4/5). As used herein, this means that the protein is capable of binding to NGF and inhibiting NGF biological activity and/or downstream pathway(s) mediated by NGF signalling. An NGF binding protein reduces NGF biological activity, including downstream pathways mediated by NGF signalling and/or reduces the amount of NGF that is in circulation, and which can bind to its receptors trkA and NGFR (p75NTR).

The term companion animal as used herein refers to a dog, cat or horse. In one embodiment, the companion animal is a dog. In another embodiment, the animal to be treated may be a cow or pig. In another embodiment, the animal to be treated may be a camel.

The term “isolated” molecule, protein or polypeptide refers to a molecule, protein or polypeptide that is substantially free of other proteins or polypeptides, having different antigenic specificities. Moreover, protein or polypeptide may be substantially free of other cellular material and/or chemicals. Thus, the protein, nucleic acids and polypeptides described herein are preferably isolated. Thus, as used herein, an “isolated” protein, or polypeptide means protein or polypeptide that has been identified and separated and/or recovered from a component of its natural cell culture environment. Contaminant components of its natural environment are materials that would interfere with diagnostic or therapeutic uses for the protein or polypeptide, and may include enzymes, hormones, and other proteinaceous or non-proteinaceous solutes.

The terms “polypeptide” and “protein” are used interchangeably to refer to a polymer of amino acid residues and are not limited to a minimum length. Peptides, oligopeptides, dimers, multimers, and the like, are also composed of linearly arranged amino acids linked by peptide bonds, and whether produced biologically, recombinantly, or synthetically and whether composed of naturally occurring or non-naturally occurring amino acids, are included within this definition. Both full-length proteins and fragments thereof are encompassed by the definition. The terms also include co-translational and post-translational modifications of the polypeptide, such as, for example, disulfide-bond formation, glycosylation, acetylation, phosphorylation, proteolytic cleavage (e.g., cleavage by furins or metalloproteases and prohormone convertases (PCs)), and the like. Furthermore, for purposes of the present invention, a “polypeptide” encompasses a protein that includes modifications, such as deletions, additions, substitutions and post-translational modifications (generally conservative in nature as would be known to a person in the art), to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods. Polypeptides or proteins are composed of linearly arranged amino acids linked by peptide bonds, but in contrast to peptides, have a well-defined conformation.

Proteins, as opposed to peptides, generally consist of chains of 50 or more amino acids. For the purposes of the present invention, the term “peptide” as used herein typically refers to a sequence of amino acids of made up of a single chain of D- or L-amino acids or a mixture of D- and L-amino acids joined by peptide bonds. Generally, peptides contain at least two amino acid residues and are less than about 50 amino acids in length.

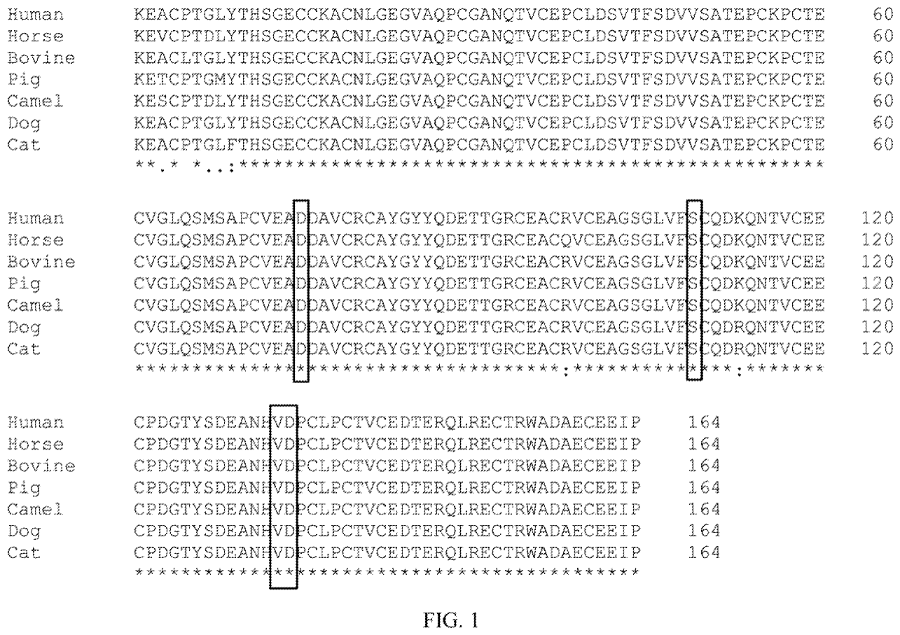

The present invention relates to molecules comprising a variant of a p75NTR extracellular domain. The variant p75NTR may be, without limitation, a human p75NTR variant, canine p75NTR variant, a feline p75NTR variant, an equine p75NTR variant, a bovine p75NTR variant, a cameline p75NTR variant, or a porcine p75NTR variant. Human, canine, feline, bovine, equine and cameline p75NTR proteins all have a very high degree of sequence similarity as shown in FIG. 1 and the conserved sequences extend to other animals as well. The variant extracellular domain of p75NTR comprises a different amino acid at one or more position within the polypeptide chain compared to that of the wild-type p75NTR. The variant amino acid may be present at one or more of position 75, 109, 133 and/or 134 of the extracellular domain of p75NTR. Positions 75, 109, 133 and 134 are highlighted in FIG. 1. Position numbering is based on the amino acid sequences of the canine, feline, equine, bovine and human p75NTR as set out in SEQ ID Nos. 1, 3, 5, 36 and 71 respectively.

The term “variant amino acid” as used herein refers to any amino acid that is not the amino acid present in the wild-type amino acid sequence, in this case the wild-type p75NTR sequence. The wild-type sequence may be a wild-type mammal p75NTR sequence. The wild-type sequence may be a wild-type human p75NTR sequence. The wild-type sequence may be the wild-type canine p75NTR sequence, wild-type feline p75NTR sequence, wild-type equine p75NTR sequence, wild-type bovine p75NTR sequence, or wild-type cameline sequence, which are disclosed herein. The wild-type canine, feline, equine, bovine and human p75NTR sequences are set out in FIG. 1 and SEQ ID Nos. 1, 3, 5, 36 and 71. When referring to the wild-type p75NTR sequence this may refer to the entire sequence or part thereof, for example the extracellular domain.

The variant amino acid may be a result of substituting, replacing, or modifying the original (e.g. wild-type or germline) amino acid, within a protein sequence, with a different amino acid. The process of substituting or replacing an amino acid can be done using standard techniques available to the skilled person, e.g. using recombinant DNA technology. Modification of an amino acid may be performed post-translationally and a variety of chemical or bioconjuagtion methods may be used to modify said amino acid. The amino acids are changed relative to the native (wild type/germline) sequence as found in nature in the wild type (WT). By “wild type” or “WT” or “native” herein is meant an amino acid sequence or a nucleotide sequence that is found in nature, including allelic variations. A wild-type protein or polypeptide has an amino acid sequence or a nucleotide sequence that has not been intentionally modified.

The term “amino acid” as used herein refers to one of the 20 naturally occurring (canonical) amino acids or any non-natural analogues (non-canonical amino acids) that may be present at a specific, defined position within a peptide sequence. “Amino acid” encompasses both naturally occurring and synthetic amino acids. Although in most cases, when the protein is to be produced recombinantly, only naturally occurring amino acids are used. The variant amino acid may comprise one of the twenty canonical amino acids. The variant amino acid may comprise a non-canonical amino acid, also known are non-natural amino acids for example hydroxyproline, hydroxylysine, phosphoserine, phosphothreonine, phosphotyrosine, N-acetyl lysine, methyllysine.

Mutant designations herein refer to a position and the amino acid replacement at that position. For example, the mutant designation E75T indicates the presence of threonine at position 75, not that the mutation was arrived at by substitution for glutamate.

The variant amino acid at position 75 may be an amino acid selected from alanine, arginine, asparagine, aspartic acid, cysteine, glutamine, glycine, histidine, isoleucine, leucine, lysine, methionine, phenylalanine, proline, serine, threonine, tryptophan, tyrosine, valine. The variant amino acid at position 109 may be selected from alanine, arginine, asparagine, aspartic acid, cysteine, glutamic acid, glutamine, glycine, histidine, isoleucine, leucine, lysine, methionine, phenylalanine, proline, threonine, tryptophan, tyrosine, valine. The variant amino acid at position 133 may be selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine. The variant amino acid at position 134 may be selected from alanine, arginine, asparagine, cysteine, glutamic acid, glutamine, glycine, histidine, isoleucine, leucine, lysine, methionine, phenylalanine, proline, serine, threonine, tryptophan, tyrosine, valine.

The variant amino acid at position 75 may be an amino acid comprising a polar side chain. For example, the variant amino acid a position 75 may be selected from serine, threonine, tyrosine, tryptophan, asparagine, glutamine or cysteine. Preferably the variant amino acid at position 75 comprises a small polar side chain for example serine or threonine. In one embodiment the variant amino acid at position 75 is threonine.

The variant amino acid at position 109 may be an amino acid comprising an aromatic side chain. For example, the variant amino acid a position 109 may be selected from histidine, tyrosine, phenylalanine, or tryptophan. In one embodiment the variant amino acid at position 109 is histidine. In one embodiment the variant amino acid at position 109 is tyrosine.

The variant amino acid at position 133 may be an amino acid comprising a charged side chain. For example, the variant amino acid a position 133 may be selected from arginine, histidine, lysine, aspartic acid, or glutamic acid. In one embodiment the variant amino acid a position 133 may be an amino acid comprising a negatively charged side chain. For example, the variant amino acid at position 133 is selected from arginine, histidine, or lysine. In one embodiment the variant amino acid at position 133 is arginine.

The variant amino acid at position 134 may be an amino acid comprising a hydrophobic side chain. For example, the variant amino acid a position 134 may be selected from alanine, valine, isoleucine, leucine, methionine, phenylalanine, tyrosine, or tryptophan. In one embodiment the variant amino acid a position 134 may be an amino acid comprising a non-aromatic hydrophobic side chain. For example the variant amino acid at position 134 is selected from alanine, valine, isoleucine, leucine, or methionine. In one embodiment the variant amino acid at position 134 is leucine or isoleucine. In one embodiment the variant amino acid is leucine.

Amino acid modifications in general refer to and include substitutions, insertions and deletions, with the former being preferred in many cases. The variant extracellular domain of p75NTR of the invention described herein, comprising a variant amino acid at one or more of positions 75, 109, 133 and/or 134, may comprise additional variant amino acids within the polypeptide sequence. These additional variant amino acids can include any number of further modifications, as long as the function of the protein is still present, as described herein. It will be clear to the skilled person that additional mutations may occur naturally within the amino acid sequence or additional mutations may be genetically engineered for example to increase stability or reduce glycosylation. In one embodiment, from 1, 2, 3, 4, 5, 6, 7, 8, 9 or 10 modifications are generally utilized as often the goal is to alter function with a minimal number of modifications. A variant polypeptide sequence will preferably possess at least 80%, at least 85%, at least 90%, at least 95%, at least 96%, at least 97%, at least 98%, or at least 99% identity to the wild-type sequences or the parent sequences. It should be noted that depending on the size of the sequence, the percent identity will depend on the number of amino acids.

By “protein variant” or “variant protein” herein is meant a protein that differs from a wild-type protein by virtue of at least one amino acid modification. The isolated polypeptide according to the invention comprises a p75 neurotrophin receptor (p75NTR) extracellular domain, comprising a variant amino acid at one or more of position 75, 109, 133 and/or 134 within said p75NTR, however the skilled person would understand that further variant amino acids may be present within said p75NTR compared to the wild-type p75NTR. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent. The variant polypeptide sequence herein will preferably possess at least about 80% identity with a parent polypeptide sequence, and most preferably at least about 90% identity, more preferably at least about 95% identity. Variants do not include human sequences.

By “parent polypeptide”, “parent protein” as used herein is meant an unmodified polypeptide that is subsequently modified to generate a variant. Said parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

The p75 neurotrophin receptor p75NTR in its native form exists as a transmembrane glycoprotein. Family members are characterised by multiple cysteine-rich domains for ligand binding, a single transmembrane sequence extracellular domain (ECD), and a non-catalytic cytoplasmic domain. As used herein, a portion of p75NTR or a portion of the ECD of p75NTR includes at least one neurotrophin binding domain.

Endogenous soluble ECD of p75NTR is produced by regulated proteolysis by α-secretase and γ-secretase that cleaves the protein near the membrane junction of the ECD. This is cleavage results in the release of the cytoplasmic domain which is free to bind NGF as a natural antagonist to NGF signalling.

In one embodiment, the extracellular domain of p75NTR according to the invention comprises the full-length extracellular domain (ECD) or part thereof. A part of the ECD of p75NTR may comprise various truncations of the full-length ECD. Truncations of the ECD of p75NTR may be produced using techniques known in the art such as recombinant DNA techniques. In one embodiment, the extracellular domain of p75NTR comprises or consists of the full-length ECD. In one embodiment, the extracellular domain of p75NTR comprises the extracellular domain (ECD) and additional C-terminal amino acids of the p75NTR protein adjacent to the ECD. For example, the portion of the p75NTR protein may comprise the ECD and at least 1-5 or 5-10 amino acids, e.g. 1, 2, 3, 4, 5, 6, 7, 8, 9 or 10 C-terminal amino acids of the p75NTR protein adjacent to the ECD; or the ECD and at least 10-20 amino acids, or the ECD and at least 20-30 amino acids, or the ECD and at least 30-40 amino acids, or the ECD and at least 40-50 amino acids, or the ECD and at least 50-60 amino acids, or the ECD and at least 60-70 amino acids, or the ECD and at least 70-80 amino acids, or the ECD and at least 80-90 amino acids, or the ECD and at least 90-100 amino acids, or the ECD and at least 100-110 amino acids, or the ECD and at least 110-120 amino acids, or the ECD and at least 120-130 amino acids, or the ECD and at least 130-140 amino acids, or the ECD and at least 140-150 amino acids, or the ECD and at least 150-160 amino acids, or the ECD and at least 160-170 amino acids or the ECD and at least 170-180 amino acids C-terminal amino acids of the companion animal p75NTR protein adjacent to the ECD.

In one embodiment, a-secretase and y-secretase cleavage sites within the ECD are removed. In one embodiment, all or a part of the stalk region is removed. As used herein, the stalk region refers to the amino acids downstream from the conserved EEIP sequence at positions 161-164. In one embodiment, the stalk region and α-secretase and γ-secretase cleavage sites within the ECD are removed. In one embodiment, a portion of the N-terminus of the mature p75NTR protein is removed, e.g., 1 2, 3, 4, 5, 6, 7, 8, 9, or 10 or more amino acids at the N-terminus. In one embodiment, a portion of the amino acids directly upstream of the stalk region are removed, e.g., 1, 2, 3, 4, 5, 6, 7, 8, 9, 10 or more contiguous amino acids.

In one embodiment, the p75NTR extracellular domain is canine and comprises or consists of SEQ ID NO:7 or a variant thereof or a portion thereof. Variants according to the invention comprise a variant amino acid at one or more of positions 75, 109, 133 and/or 134, for example SEQ ID NO:46, SEQ ID NO:49, SEQ ID NO:52, SEQ ID NO:55 or SEQ ID NO:58. In an embodiment the isolated polypeptide comprising a companion animal p75 neurotrophin receptor (p75NTR) extracellular domain, wherein said p75NTR comprises a variant amino acid at one or more of position 75, 109, 133 and/or 134 comprises or consists of SEQ ID NO:46, SEQ ID NO:49, SEQ ID NO:52, SEQ ID NO:55 or SEQ ID NO:58.

In another embodiment, the isolated companion animal p75NTR extracellular domain is feline and comprises or consists of SEQ ID NO:38 or a variant thereof or a portion thereof. Variants according to the invention comprise a variant amino acid at one or more of positions 75, 109, 133 and/or 134 for example SEQ ID NO:61, SEQ ID NO:63, SEQ ID NO:65, SEQ ID NO:67 or SEQ ID NO:69. In an embodiment the isolated polypeptide comprises a companion animal p75 neurotrophin receptor (p75NTR) extracellular domain, wherein said p75NTR comprising a variant amino acid at one or more of position 75, 109, 133 and/or 134 comprises or consists of SEQ ID NO:61, SEQ ID NO:63, SEQ ID NO:65, SEQ ID NO:67 or SEQ ID NO:69.

In another embodiment, the isolated p75NTR extracellular domain is equine and comprises or consists of SEQ ID NO:118 or a variant thereof or a portion thereof. Variants according to the invention comprise a variant amino acid at one or more of positions 75, 109, 133 and/or 134 for example SEQ ID NO:120, SEQ ID NO:121, SEQ ID NO:122, SEQ ID NO:123 or SEQ ID NO:124. In an embodiment the isolated polypeptide comprises an equine p75 neurotrophin receptor (p75NTR) extracellular domain, wherein said p75NTR comprising a variant amino acid at one or more of position 75, 109, 133 and/or 134 comprises or consists of SEQ ID NO:120, SEQ ID NO:121, SEQ ID NO:122, SEQ ID NO:123 or SEQ ID NO:124.

In another embodiment, the isolated p75NTR extracellular domain is bovine and comprises or consists of SEQ ID NO:125 or a variant thereof or a portion thereof. Variants according to the invention comprise a variant amino acid at one or more of positions 75, 109, 133 and/or 134 for example SEQ ID NO:127, SEQ ID NO:128, SEQ ID NO:129, SEQ ID NO:130 or SEQ ID NO:131. In an embodiment the isolated polypeptide comprises a bovine p75 neurotrophin receptor (p75NTR) extracellular domain, wherein said p75NTR comprising a variant amino acid at one or more of position 75, 109, 133 and/or 134 comprises or consists of SEQ ID NO:127, SEQ ID NO:128, SEQ ID NO:129, SEQ ID NO:130 or SEQ ID NO:131.

In another embodiment, the isolated p75NTR extracellular domain is cameline and comprises or consists of SEQ ID NO:101 or a variant thereof or a portion thereof. Variants according to the invention comprise a variant amino acid at one or more of positions 75, 109, 133 and/or 134 for example SEQ ID NO:103, SEQ ID NO:104, SEQ ID NO:105, SEQ ID NO:106 or SEQ ID NO:107. In an embodiment the isolated polypeptide comprises a companion animal p75 neurotrophin receptor (p75NTR) extracellular domain, wherein said p75NTR comprising a variant amino acid at one or more of position 75, 109, 133 and/or 134 comprises or consists of SEQ ID NO:103, SEQ ID NO:104, SEQ ID NO:105, SEQ ID NO:106 or SEQ ID NO:107.

In another embodiment, the isolated p75NTR extracellular domain is porcine and comprises or consists of SEQ ID NO:110 or a variant thereof or a portion thereof. Variants according to the invention comprise a variant amino acid at one or more of positions 75, 109, 133 and/or 134 for example SEQ ID NO:112, SEQ ID NO:113, SEQ ID NO:114, SEQ ID NO:115 or SEQ ID NO:116. In an embodiment the isolated polypeptide comprises a companion animal p75 neurotrophin receptor (p75NTR) extracellular domain, wherein said p75NTR comprising a variant amino acid at one or more of position 75, 109, 133 and/or 134 comprises or consists of SEQ ID NO:112, SEQ ID NO:113, SEQ ID NO:114, SEQ ID NO:115 or SEQ ID NO:116.

In another embodiment, the isolated p75NTR extracellular domain is human and comprises or consists of SEQ ID NO:71 or a variant thereof or a portion thereof. Variants according to the invention comprise a variant amino acid at one or more of positions 75, 109, 133 and/or 134 for example SEQ ID NO:84, SEQ ID NO:87, SEQ ID NO:90, SEQ ID NO:93 or SEQ ID NO:96. In an embodiment the isolated polypeptide comprising a human p75 neurotrophin receptor (p75NTR) extracellular domain, wherein said p75NTR comprises a variant amino acid at one or more of position 75, 109, 133 and/or 134 comprises or consists of SEQ ID NO:84, SEQ ID NO:87, SEQ ID NO:90, SEQ ID NO:93 or SEQ ID NO:96.

The ECD of p75NTR has a stalk region (e.g. SEQ ID NO:9, canine stalk region) that is prone to O-glycosylation. Glycosylation in proteins can cause manufacturing difficulties. Thus, in one embodiment, the isolated ECD may comprise deletions in the stalk region to reduce the number of O-glycosylation sites within the stalk region e.g. to form a truncated stalk region. A truncated stalk region may comprise any number of the amino acids of the stalk region. For example, the stalk region may comprise 1-10, 1-20, 1-30 amino acids. The stalk region may be removed in embodiments described herein. Point mutations may be introduced into the stalk region to produce a stalk region with a reduced number of o-glycosylation sites. For example point mutations may be used to introduced variant amino acids at the o-glycosylation sites to prevent glycosylation occurring at these sites. Thus, a portion of the ECD as used herein may be the ECD without the stalk region and 3′ sequences α-secretase and γ-secretase cleavage sites (e.g. SEQ ID NO:34).

Thus, in one embodiment, the ECD of the p75NTR is a truncated protein which has the O-glycosylation stalk region removed.

The isolated polypeptide comprising a p75NTR ECD wherein said p75NTR comprises a variant amino acid at one or more of position 75, 109, 133 and/or 134, has an altered binding affinity for its target compared to the wild-type p75NTR ECD. The p75NTR ECD of the invention may have an altered binding affinity for one or more or NGF, BDNF, NT3, NT4 compared to the wild-type p75NTR ECD. In an embodiment the binding affinity of the variant p75NTR ECD of the invention is increased for one or more or NGF, BDNF, NT3, NT4 compared to the wild-type p75NTR ECD. In an embodiment the binding affinity of the variant p75NTR ECD of the invention is decreased for one or more or NGF, BDNF, NT3, NT4 compared to the wild-type p75NTR ECD.

In an embodiment isolated polypeptide comprising a p75NTR ECD, wherein said p75NTR comprises a variant amino acid at one or more of position 75, 109, 133 and/or 134 comprises an altered binding affinity for NGF compared to the wild-type p75NTR ECD. In an embodiment the p75NTR ECD of the invention comprises an altered binding affinity for NGF compared to the wild-type p75NTR ECD but retains a similar binding affinity for BDNF, NT3 and/or NT4. In an embodiment the binding affinity of the variant p75NTR ECD is increased for NGF compared to the wild-type p75NTR ECD. In an embodiment the binding affinity of the variant p75NTR ECD of the invention is decreased for NGF compared to the wild-type p75NTR ECD.

### Nucleic Acids

In another aspect, the invention relates to an isolated nucleic acid encoding the isolated polypeptide comprising a p75 neurotrophin receptor (p75NTR) extracellular domain, wherein said p75NTR comprises a variant amino acid at one or more of position 75, 109, 133 and/or 134 within said p75NTR.

In one embodiment, the p75NTR is from a human and the isolated nucleic acid encodes SEQ ID NO:84, SEQ ID NO:87, SEQ ID NO:90, SEQ ID NO:93 or SEQ ID NO:96. In one embodiment, the companion animal is a dog and the isolated nucleic acid encodes SEQ ID NO:46, SEQ ID NO:49, SEQ ID NO:52, SEQ ID NO:55 or SEQ ID NO:58. In one embodiment the companion animal is a cat and the isolated nucleic acid encodes SEQ ID NO:61, SEQ ID NO:63, SEQ ID NO:65, SEQ ID NO:67 or SEQ ID NO:69. In one embodiment, the p75NTR is from a horse and the isolated nucleic acid encodes SEQ ID NO:120, SEQ ID NO:121, SEQ ID NO:122, SEQ ID NO:123 or SEQ ID NO:124. In one embodiment, the p75NTR is from a cow and the isolated nucleic acid encodes SEQ ID NO:127, SEQ ID NO:128, SEQ ID NO:129, SEQ ID NO:130 or SEQ ID NO:131. In one embodiment, the p75NTR is from a pig and the isolated nucleic acid encodes SEQ ID NO:112, SEQ ID NO:113, SEQ ID NO:114, SEQ ID NO:115 or SEQ ID NO:116. In one embodiment, the p75NTR is from a camel and the isolated nucleic acid encodes SEQ ID NO:103, SEQ ID NO:104, SEQ ID NO:105, SEQ ID NO:106 or SEQ ID NO:107.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature.

In another aspect, the invention relates to a vector, plasmid, transcription, expression cassette or nucleic acid construct comprising a nucleic acid encoding the ECD of a p75NTR, e.g. the ECD or portion thereof as described above.

The construct may include a suitable leader sequence. The term leader sequence is used interchangeably with signal sequence. Thus, in some embodiments, the nucleic acid sequence/nucleic acid construct encoding the fusion protein may also comprise a leader sequence. The leader sequence is made as part of the protein and then cleaved off when the protein is secreted. Any suitable leader sequence may be used, including a native immunoglobulin germline leader sequence, such as the endogenous p75 leader of the relevant species (e.g. human, canine, equine, feline, bovine, cameline), the endogenous p75 leader of a different species e.g. a mouse IgG leader or another leader sequences known in the art, e.g. the Campath leader sequence (see U.S. Pat. No. 8,362,208 B2) or an artificial sequence. Such leader sequences can aid in enhancing protein expression.

In another aspect, the invention relates to a host cell comprising a nucleic acid encoding an ECD of a human, companion animal, or other p75NTR, e.g. the ECD, or a vector, plasmid, vector, transcription, expression cassette or construct as described above.

Expression vectors of use in the invention may be constructed from a starting vector such as a commercially available vector. After the vector has been constructed and the nucleic acid molecule has been inserted into the proper site of the vector, the completed vector may be inserted into a suitable host cell for amplification and/or polypeptide expression.

The term “vector” means a construct, which is capable of delivering, and in some aspects expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The invention also relates to an isolated recombinant host cell comprising one or more nucleic acid molecule plasmid, vector, transcription or expression cassette as described above. The transformation of an expression vector into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

The host cell may be eukaryotic or prokaryotic, for example a bacterial, viral, plant, fungal, mammalian or other suitable host cell. In one embodiment, the cell is an E. coli cell. In another embodiment, the cell is a yeast cell. In another embodiment, the cell is a Chinese Hamster Ovary (CHO) cell, HeLa cell or other cell that would be apparent to the skilled person. Mammalian cell lines available as hosts for expression are well known in the art and include, but are not limited to, immortalized cell lines available from the American Type Culture Collection (ATCC) and any cell lines used in an expression system known in the art can be used to make the recombinant polypeptides of the invention.

In general, host cells are transformed with a recombinant expression vector that comprises DNA encoding a protein. Among the host cells that may be employed are prokaryotes, yeast or higher eukaryotic cells. Prokaryotes include gram negative or gram-positive organisms, for example E. coli or bacilli. Higher eukaryotic cells include insect cells and established cell lines of mammalian origin. Examples of suitable mammalian host cell lines include the COS-7 cells, L cells, CI27 cells, 3T3 cells, Chinese hamster ovary (CHO) cells, or their derivatives and related cell lines which grow in serum free media, HeLa cells, BHK cell lines, the CVIIEBNA cell line, human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays.

Other suitable host cells include insect cells, using expression systems such as baculovirus in insect cells, plant cells, transgenic plants and transgenic animals, and by viral and nucleic acid vectors.

Alternatively, it is possible to produce the polypeptide in lower eukaryotes such as fungal cell lines and yeast or in prokaryotes such as bacteria. Suitable yeasts include S. cerevisiae, S. pombe, Kluyveromyces strains, Pichia pastoris, Candida, or any yeast strain capable of expressing heterologous polypeptides. Suitable bacterial strains include E. coli, B. subtilis, S. typhimurium, or any bacterial strain capable of expressing heterologous polypeptides. If the protein is made in yeast or bacteria, it may be desirable to modify the product produced therein, for example by phosphorylation or glycosylation of the appropriate sites, in order to obtain a functional product. Such covalent attachments can be accomplished using known chemical or enzymatic methods.

A host cell, when cultured under appropriate conditions, can be used to express a protein that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule.

In another aspect, the invention also relates to the use of an isolated p75NTR protein or a portion thereof as described above in a fusion protein with another moiety, e.g. with a half-life extending moiety as described in more detail below. Therefore, the p75NTR protein or a portion thereof can be provided covalently linked or couple to a half-life extending moiety. Alternatively, it may be provided incorporated in a liposome. The invention further relates to an isolated p75NTR protein or a portion thereof for use in therapy. Further, there is provided an isolated p75NTR protein or a portion thereof for use in the treatment of a pain related disease. Such diseases are described in more detail below.

In some embodiments, to improve its pharmacokinetic (PK) properties, the half-life of the p75NTR protein is extended.

### Fusion Proteins

Thus, in another aspect, the invention relates to a fusion protein comprising a p75 neurotrophin receptor (p75NTR) extracellular domain, comprising a variant amino acid at one or more of position 75, 109, 133 and/or 134 within said p75NTR, and another moiety. The fusion protein may comprise an extracellular domain of a p75 neurotrophin receptor (p75NTR), wherein said p75NTR comprises a variant amino acid at one or more of position 75, 109, 133 and/or 134, and another moiety.

For example, the other moiety may be a half-life extending moiety. Thus, the p75NTR protein or portion thereof (e.g., extracellular domain) is coupled to a half-life extending moiety. As described above, the p75NTR protein or portion thereof may be human, canine, feline, bovine, equine, or cameline. The p75NTR protein or portion thereof used in the fusion protein may thus comprise or consist of a sequence selected from SEQ ID NO:1, 3, 5, 7, 34, 36, 38, 71, or 73 or a portion or a variant thereof, in particular the variant may comprise a variant amino acid at one or more of position 75, 109, 133 and/or 134. In certain embodiments, all or part of the stalk region of the p75NTR is removed. In certain embodiments of the canine p75NTR protein or portion, the stalk region (e.g. SEQ ID NO:9) is removed. In certain embodiments of the feline p75NTR protein or portion, the stalk region is removed. In certain embodiments of the human p75NTR protein or portion, the stalk region (e.g., SEQ ID NO:25) is removed.

Half-life extending moieties have been described. For example, the half-life extending moiety may be selected from the following non-limiting list: a human immunoglobulin Fc domain, a companion animal immunoglobulin Fc domain, polyethylene glycol (PEG), PEG derivatives, simple lipids, lipid dicarboxylic acids, lipids with additional moieties, human or companion animal serum albumin binders, e.g. small-molecule binders or antibodies/antibody fragments that bind human or companion animal serum albumin, companion animal serum albumin, or streptococcal protein G's albumin-binding domain (ABD). Examples of lipids include glucagon-like peptide 1 (GLP-1), the analogs GLP-1 liraglutide and semaglutide or cholesterol. Advantageously, using an immunoglobulin Fc domain facilitates purification of the protein. In particular, Fc binding to Protein A can be used in purification procedures. The presence of an immunoglobulin Fc domain can also stabilise the overall folding of the fusion protein as well as extending its half-life.

In certain embodiments, where the half-life extending moiety is an Fc domain, serum albumin binder, or serum albumin, the extracellular domain of p75NTR and half-life extending moiety are from/specific to the same species. For example, in one embodiment, the half-life extending moiety is a companion animal Fc domain of the corresponding companion animal. For example, if the extracellular domain of p75NTR is canine, the Fc domain is canine. If the extracellular domain of p75NTR is feline, the Fc domain is feline. If the extracellular domain of p75NTR is equine, the Fc domain is equine. If the extracellular domain of p75NTR is bovine, the Fc domain is bovine.

In one embodiment, where the half-life extending moiety is an Fc domain, human serum albumin binder or human serum albumin, the extracellular domain of p75NTR and half-life extending moiety are both derived from human.

The human serum albumin binder, e.g. antibody of fragment thereof, may be fully human or humaized. The human serum albumin binder binds to human serum albumin.

However, given the high sequence similarity between p75 proteins, in certain embodiments, where the half-life extending moiety is a companion animal Fc domain, companion animal serum albumin binder or companion animal serum albumin, the p75NTR protein or portion and half-life extending moiety are not from/specific to the same companion animal. For example, in one embodiment, the half-life extending moiety is the companion animal Fc domain of the corresponding companion animal, but the p75 protein or portion thereof is that of a different companion animal. For example, for treatment of dogs, if the Fc domain is canine, the p75NTR protein or portion thereof, e.g. the extracellular domain is may be from a different animal, e.g. cat, cow, horse, pig, or camel. For example, for treatment of cats, if the Fc domain is feline, the p75NTR protein or portion thereof, e.g. the extracellular domain is may be from a different animal, e.g. dog, cow, pig, horse, or camel. For example, for treatment of cats, if the Fc domain is equine, the p75NTR protein or portion thereof, e.g. the extracellular domain is may be from a different animal, e.g. cat, cow, dog, pig, or camel. In yet another embodiment, human p75 or a portion thereof fused to companion animal Fc can be used.

The companion animal serum albumin binder, e.g. antibody or fragment thereof, may be canine or caninized, feline or felinized, equine or equinized, bovine or bovinized, camel or camelized. The companion animal serum albumin binder may bind to canine, feline, bovine, equine, or cameline serum albumin.

In one embodiment, the half-life extending moiety is a wild type or variant Fc domain. The term variant is as defined above. For example, an Fc domain variant may have modified half-life compared to the wild type Fc domain. In one embodiment, the Fc domain is a Fc domain, that is a wild-type domain or a variant thereof. Variant Fc domains are described, for example in WO2020/142625.

By “Fc” or “Fc region” or “Fc domain” as used herein is meant the polypeptide comprising the constant region of an antibody excluding the first constant region immunoglobulin domain (CH1) and, in some cases, part of the hinge. In one embodiment, the Fc domain includes constant region immunoglobulin domains CH2, CH3 and the hinge region between CH1 and CH2 or part of the hinge region.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). Exemplary amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Amino acid sequences for IgG-A, IgG-B, IgG-C and IgG-D as used by the inventors and according to the aspects and embodiments of the invention are provided as SEQ ID Nos. 15, 16, 17, 18).

In human, Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains CH2 and CH3 and the lower hinge region between CH1 and CH2. Although the boundaries of the Fc region may vary, the human IgG heavy chain Fc region is usually defined to include residues C226 or P230 to its carboxyl-terminus, wherein the numbering is according to the EU index as in Kabat.

Fc as used herein may refer to the Fc region in isolation, or this region in the context of an Fc fusion (“fusion composition” or “fusion construct”), as described herein. Fc domains include all or part of an Fc region; that is, N- or C- terminal sequences may be removed from wild-type or variant Fc domains, as long as this does not affect function.

Briefly, IgG functions are generally achieved via interaction between the Fc region of the Ig and an Fcγ receptor (FcγR) or another binding molecule, sometimes on an effector cell. This can trigger the effector cells to kill target cells to which the antibodies are bound through their variable (V) regions. Also, antibodies directed against soluble antigens might form immune complexes which are targeted to FcγRs which result in the uptake (opsonisation) of the immune complexes or in the triggering of the effector cells and the release of cytokines.

By “Fc gamma receptor”, “ FcγR ” or “FcgammaR” as used herein is meant any member of the family of proteins that bind the IgG antibody Fc region and is encoded by an FcγR gene.

In humans, three classes of FcγR have been characterised, although the situation is further complicated by the occurrence of multiple receptor forms. The three classes are:
(i) FcγRI (CD64) including isoforms FcγRIa, FcγRIb, and FcγRIc binds monomeric IgG with high affinity and is expressed on macrophages, monocytes, and sometimes neutrophils and eosinophils;
(ii) FcγRII (CD32) binds complexed IgG with medium to low affinity and is widely expressed. These receptors can be divided into two important types, FcγRIIa and FcγRIIb. The ‘a’ form of the receptor is found on many cells involved in killing (e. g. macrophages, monocytes, neutrophils) and seems able to activate the killing process and occurs as two alternative alleles. The ‘b’ form seems to play a role in inhibitory processes and is found on B-cells, macrophages and on mast cells and eosinophils. On B-cells it seems to function to suppress further immunoglobulin production and isotype switching to for example, the IgE class. On macrophages, the b form acts to inhibit phagocytosis as mediated through FcγRIIa. On eosinophils and mast cells the b form may help to suppress activation of these cells through IgE binding to its separate receptor and
(iii) FcyRIII (CD16) binds IgG with medium to low affinity and exists as two types. FcγRIIIa is found on NK cells, macrophages, eosinophils and some monocytes and T cells and mediates ADCC.

FcγRIIIb is highly expressed on neutrophils. Both types have different allotypic forms.

Canine Fc receptors are described in Bergeron et al L.M. Bergeron et al.; Veterinary Immunology and Immunopathology 157 (2014) 31-41. Canine has RI, RIIb, RIII, but not Riia.

As well as binding to FcγRs, IgG antibodies can activate complement and this can also result in cell lysis, opsonisation or cytokine release and inflammation. The Fc region also mediates such properties as the transportation of IgGs to the neonate (via the so-called “FcRn”), increased half-life (also believed to be effected via an FcRn-type receptor) and self-aggregation. The Fc-region is also responsible for the interaction with protein A and protein G (which interaction appears to be analogous to the binding of FcRn).

By “effector function” as used herein is meant a biochemical event that results from the interaction of an antibody Fc region with an Fc receptor or ligand. Effector functions include but are not limited to antibody-dependent cell-mediated cytotoxicity (ADCC), antibody-dependent cell-mediated phagocytosis (ADCP) and complement-dependent cytotoxicity (CDC).

In one embodiment, the ECD of p75NTR, and the other moiety are linked with a linker moiety or otherwise conjugated, attached or covalently or non-covalently linked. Suitable linkers are known to the skilled person. For example, the linker is a peptide linker, such as a glycine and/or alanine and/or threonine and/or serine-rich linker e.g. a glycine-serine linker, such as (G4S)n wherein n is 1 to 4.

In another embodiment, the linker can be cleavable.

In one embodiment of the fusion protein, the companion animal p75NTR protein or portion thereof comprises or consists of a variant canine p75NTR ECD or portion thereof. In one embodiment, the ECD is canine and comprises of consists of a sequence selected from SEQ ID NO:46, SEQ ID NO:49, SEQ ID NO:52, SEQ ID NO:55 or SEQ ID NO:58. In one embodiment the ECD is feline and comprises or consists of a sequence selected from SEQ ID NO:61, SEQ ID NO:63, SEQ ID NO:65, SEQ ID NO:67 or SEQ ID NO:69. In one embodiment the ECD is human and comprises or consists of a sequence selected from SEQ ID NO:84, SEQ ID NO:87, SEQ ID NO:90, SEQ ID NO:93 or SEQ ID NO:96.

Thus, in one embodiment, the invention relates to a fusion protein comprising a canine p75NTR ECD linked to a canine Fc domain. In one embodiment, the ECD comprises of consists of a sequence selected from SEQ ID NO:46, SEQ ID NO:49, SEQ ID NO:52, SEQ ID NO:55 or SEQ ID NO:58 or a variant thereof operably linked to a canine Fc domain. In one embodiment, the invention relates to a fusion protein comprising a feline p75NTR ECD linked to a feline Fc domain. In one embodiment, the ECD comprises of consists of a sequence selected from SEQ ID NO:61, SEQ ID NO:63, SEQ ID NO:65, SEQ ID NO:67 or SEQ ID NO:69 or a variant thereof operably linked to a feline Fc domain. In one embodiment, the invention relates to a fusion protein comprising a human p75NTR ECD linked to a human Fc domain. In one embodiment, the ECD comprises of consists of a sequence selected from SEQ ID NO:84, SEQ ID NO:87, SEQ ID NO:90, SEQ ID NO:93 or SEQ ID NO:96 or a variant thereof operably linked to a human Fc domain.

In one embodiment, the fusion protein of the present invention preferably binds to any one or more of NGF, BDNF, NT3 or NT4/5 with a binding affinity (Kd) of between about 1 pM to about 100 nM. In some preferred embodiments, the binding affinity (Kd) is between about 5 pM and any of about 10 pM, 20 pM, 40 pM, 50 PM 100 PM, 0.2 nM, 0.5 nM, 1 nM 1.5 nM 2 nM, 2.5 nM, 3 nM, 3.5 nM, 4 nM, 4.5 nM, 5 nM, 5.5 nM, 6 nM, 6.5 nM, 7 nM, 7.5 nM, 8 nM, 8.5 nM, 9 nM, 9.5 nM, 10 nM, 15 nM, 20 nM, 25 nM, 30 nM, 35 nM, 40 nM, 45 nM, 50 nM or 100 nM as measured in an in vitro binding assay for NGF, BDNF, NT3 or NT4/5 such as described herein. Subnanomolar range is preferred.

In one embodiment, the fusion protein comprises SEQ ID NO:46, SEQ ID NO:49, SEQ ID NO:52, SEQ ID NO:55 or SEQ ID NO:58 or a variant thereof. Fusion proteins according to the invention may include a variant p75NTR ECD with a variant amino acid at one or more of position 75, 109, 133 and/or 134, operably linked to a canine Fc domain. The Fc domain in the fusion protein construct may be a wild type canine Fc domain such as SEQ ID NO:20. The Fc domain in the fusion protein construct may be a variant canine Fc domain which has been modified to increase half-life for example SEQ ID NO:21. In this domain, the mutation YTE has been introduced at residues Y252-T254 of the wt Fc domain using EU numbering. The fusion protein may be generated by combining a variant p75NTR ECD such as SEQ ID NO:46, SEQ ID NO:49, SEQ ID NO:52, SEQ ID NO:55 or SEQ ID NO:58 with a canine Fc domain such as SEQ ID NO:20 or 21. The fusion protein may comprise or consist of a sequence selected from SEQ ID NO:47, SEQ ID NO:50, SEQ ID NO:53, SEQ ID NO:56 or SEQ ID NO:59.

In an embodiment the fusion protein comprises a wild-type feline p75NTR ECD operably linked to a feline Fc. In an embodiment, the fusion protein comprises or consists of SEQ ID NO:39, such a fusion protein includes the wild-type feline p75 ECD operably linked to a feline Fc domain. The Fc domain in the construct of SEQ ID NO:39 is a wild type feline IgG2 Fc domain. In an embodiment, the fusion protein comprises or consists of SEQ ID NO:42, such a fusion protein includes the feline p75 ECD operably linked to a feline Fc domain. The Fc domain in the construct of SEQ ID NO:42 is a wild type feline IgG1 Fc domain. In another embodiment, the fusion protein comprises or consists of SEQ ID NO:44, such a fusion protein includes the feline p75 ECD operably linked to a feline Fc domain. The Fc domain in the construct of SEQ ID NO:44 is a wild type feline IgG3 Fc domain.

In one embodiment, the fusion protein comprises SEQ ID NO:61, SEQ ID NO:63, SEQ ID NO:65, SEQ ID NO:67 or SEQ ID NO:69 or a variant thereof. Fusion proteins according to the invention may include a variant feline p75NTR ECD with a variant amino acid at one or more of position 75, 109, 133 and/or 134, operably linked to a feline Fc domain. The Fc domain in the fusion protein construct may be a wild type feline Fc domain such as SEQ ID NO:24, 25, 26 or 41. For example the fusion protein may be generated by combining a variant p75NTR ECD such as SEQ ID NO:61, SEQ ID NO:63, SEQ ID NO:65, SEQ ID NO:67 or SEQ ID NO:69 with a feline Fc domain such as SEQ ID NO:24, SEQ ID NO:25, SEQ ID NO:26 or SEQ ID NO:41. The Fc domain in the fusion protein construct may be a variant feline Fc domain which has been modified to increase half-life, the skilled person would be able to determine suitable half-life extending variants. The fusion protein may comprise or consist of a sequence selected from SEQ ID NO:62, SEQ ID NO:64, SEQ ID NO:66, SEQ ID NO:68 or SEQ ID NO:70.

Modified companion animal Fc domains that include a half-life extending mutation or mutations, e.g. canine, feline or equine Fc domains, can be used in the fusion proteins of the invention. A skilled person would know that any other known mutations that increase half-life could also be introduced in the Fc domain.

In one embodiment, the fusion protein comprises or consists of SEQ ID NO:85, SEQ ID NO:88, SEQ ID NO:91, SEQ ID NO:94 or SEQ ID NO:97 or a variant thereof. Fusion proteins according to the invention may include a variant p75NTR ECD with a variant amino acid at one or more of position 75, 109, 133 and/or 134, operably linked to a human Fc domain. The Fc domain in the fusion protein construct may be a wild type human Fc domain such as SEQ ID NO:77, SEQ ID NO:78, SEQ ID NO:79, or SEQ ID NO:80. The Fc domain in the fusion protein construct may be a variant human Fc domain which has been modified to increase half-life. Suitable modifications to increase the half-life of a human Fc domain are known in the art. The fusion protein may be generated by combining a variant p75NTR ECD such as SEQ ID NO:84, SEQ ID NO:87, SEQ ID NO:90, SEQ ID No.93 or SEQ ID NO:96 with a human Fc domain such as SEQ ID NO:77, SEQ ID NO:78, SEQ ID NO:79, or SEQ ID NO:80. The fusion protein may comprise or consist of a sequence selected from SEQ ID NO:85, SEQ ID NO:88, SEQ ID NO:91, SEQ ID NO:94 or SEQ ID NO:97.

According to the present invention, the fusion proteins demonstrate advantageous biological properties including improved solubility, stability and/or improved serum half-life. The examples show that the described molecules are very stable in both temperature and chemical stress, showing unfolding only when incubated at temperature higher than 70° C. with no aggregation up to 95° C. with Tm1 around 67° C. Improved half-life allows for less frequent dosing (a single administration in comparison to existing treatments where daily administration is required). This effect is demonstrated whilst showing strong analgesic effects. In certain embodiments, the fusion protein of the invention has a half-life in-vivo of about or more than any one of 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 1 10, 1 12, 1 14, 1 16, 1 18, 120, 122, 124, 126, 128, 130, 132, 134, 136, 138, 140, 142, 144, 146, 148, 150, 152,154, 156, 158, 160, 62, 164, 166, 168, 170, 172, 174, 176, 178, 180, 182, 184, 186, 188, 190, 192, 194, 196, 198, 200, 202, 204, 206, 208 or 210 hours +/−1 hour, further preferably the p75NTR(NBP)-Fc fusion protein of the invention has a half-life in-vivo of about or more than 24 hours.

In another embodiment, the fusion protein of the invention has a half-life in-vitro of about or more than any one of 2, 4, 6, 8,10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 62, 164, 166, 168, 170, 172, 174, 176, 178, 180, 182, 184, 186, 188, 190, 192, 194, 196, 198, 200, 202, 204, 206, 208 or 210 days +/−1 day, further the fusion protein has a half-life in-vitro of about or more than 6 days or more than 1 month. In one embodiment, the half life is 14 days.

According to the foregoing preferred embodiments, the in-vivo half-life can be the half-life in rat or in the corresponding companion animal, e.g. in a dog or cat or horse, or in a human.

Fusion proteins of the invention can operate at a very low dose, but are highly efficacious.

According to the present invention, the fusion proteins display a good safety profile. This is, for example, where subjects maintain normal body weight and haematological parameters and do not generate anti-Drug Antibodies after administration of fusion proteins.

In another aspect, the invention relates to an isolated nucleic acid encoding a fusion protein as described above, for example a nucleic acid encoding a fusion protein such as SEQ ID NO:47, SEQ ID NO:50, SEQ ID NO:53, SEQ ID NO:56 or SEQ ID NO:59, SEQ ID NO:60, SEQ ID NO:62, SEQ ID NO:64, SEQ ID NO:66, SEQ ID NO:68, SEQ ID NO:70, SEQ ID NO:85, SEQ ID NO:88, SEQ ID NO:91, SEQ ID NO:94 or SEQ ID NO:97. In one embodiment, the nucleic acid comprises or consists of a sequence selected from SEQ ID NO:48, SEQ ID NO:51, SEQ ID NO:54, SEQ ID NO:57 or SEQ ID NO:60. In one embodiment, the nucleic acid comprises or consists of a sequence selected from SEQ ID NO:86, SEQ ID NO:89, SEQ ID NO:92, SEQ ID NO:95, or SEQ ID NO:98.

In another aspect, the invention relates to a vector, plasmid, vector, transcription, expression cassette or construct comprising a nucleic acid described above.

In another aspect, the invention relates to a host cell comprising a nucleic acid vector, plasmid, vector, transcription, expression cassette or construct as described above. Suitable host cells are described elsewhere herein.

In another embodiment, the P75NTR protein, portion thereof or fusion protein is labelled with a detectable or functional label. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

### Pharmaceutical Compositions

In another aspect, there is provided a pharmaceutical composition comprising an isolated polypeptide comprising a p75 neurotrophin receptor (p75NTR) extracellular domain, comprising a variant amino acid at one or more of position 75, 109, 133 and/or 134 within said p75NTR, or a fusion protein of the invention. The fusion protein or pharmaceutical composition described herein can be administered by any convenient route, including but not limited to oral, topical, parenteral, sublingual, rectal, vaginal, ocular, intranasal, pulmonary, intradermal, intravitrial, intratumoural, intramuscular, intraperitoneal, intravenous, subcutaneous, intracerebral, transdermal, transmucosal, by inhalation, or topical, particularly to the ears, nose, eyes, or skin or by inhalation. In another embodiment, delivery is of the nucleic acid encoding the drug, e.g. a nucleic acid encoding the molecule of the invention is delivered.

Parenteral administration includes, for example, intravenous, intramuscular, intraarterial, intraperitoneal, intranasal, rectal, intravesical, intradermal, topical, intra-articular or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the polypeptide of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition can be in the form of a liquid, e.g., a solution, syrup, solution, emulsion or suspension. The liquid can be useful for oral administration or for delivery by injection, infusion (e.g., IV infusion) or sub-cutaneous.

When intended for oral administration, the composition can be in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units.

In specific embodiments, it can be desirable to administer the composition locally to the area in need of treatment, or by intravenous injection or infusion.

The amount of the polypeptide, fusion protein or pharmaceutical composition described herein that is effective/active in the treatment of a particular disease or condition will depend on the nature of the disease or condition and can be determined by standard clinical techniques. In addition, in vitro or in vivo assays can optionally be employed to help identify optimal dosage ranges. The precise dose to be employed in the compositions will also depend on the route of administration, and the seriousness of the disease or disease, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of a polypeptide of the present invention by weight of the composition. When intended for oral administration, this amount can be varied to range from about 0.1% to about 80% by weight of the composition. Preferred oral compositions can comprise from about 4% to about 50% of the polypeptide of the present invention by weight of the composition.

Compositions can be prepared so that a parenteral dosage unit contains from about 0.01% to about 2% by weight of the polypeptide of the present invention.

For administration by injection, the composition can comprise from about typically about 0.1 mg/kg to about 250 mg/kg of the subject's body weight, preferably, between about 0.1 mg/kg and about 20 mg/kg of the subject's body weight, and more preferably about 1 mg/kg to about 10 mg/kg of the subject's body weight. In one embodiment, the composition is administered at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks or more.

Treatment can for example be once a month or bi-monthly. This is advantageous over daily administration as this improves compliance and minimises stress to the subject.

As used herein, “treat”, “treating” or “treatment” means inhibiting or relieving a disease or disease. For example, treatment can include a postponement of development of the symptoms associated with a disease or disease, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the subjects being treated. Many medical treatments are effective for some, but not all, patients that undergo the treatment.

The term “subject” or “patient” refers to a human or animal which is the object of treatment, observation, or experiment, suitably a human or a companion animal, such as a canine or a feline.

In another aspect, the invention relates to the use of an isolated polypeptide comprising a p75NTR protein or portion thereof, fusion protein or pharmaceutical composition described herein in the treatment or prevention of a disease. In another aspect, the disclosure relates to the use of a polypeptide, fusion protein or pharmaceutical composition described herein in the manufacture of a medicament for the treatment or prevention of a disease as listed herein. The invention further relates to a method of treating a disease in a subject comprising an effective amount of the polypeptide, fusion protein or pharmaceutical composition as described herein to said subject. The invention further relates to an isolated polypeptide comprising a p75NTR protein described herein or a fusion protein described herein for use in the treatment or prevention of a disease listed herein.

For example, the disease is a NGF related disorder.

In one embodiment, the NGF related disorder is selected from the group consisting of: cardiovascular diseases, atherosclerosis, obesity, type 2 diabetes, metabolic syndrome, pain and inflammation. In one embodiment, the NGF related disorder comprises pain. In one embodiment, the pharmaceutical composition is used in the treatment of pain. In one embodiment, the pharmaceutical composition is used for the treatment of a pain and the type of pain is selected from osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, and gastrointestinal pain. In one embodiment, the pain comprises osteoarthritis pain. In one embodiment, the pain comprises surgical and post-surgical pain. In one embodiment, the pain comprises cancer pain.

In one or more embodiments, the isolated polypeptide comprising a p75 neurotrophin receptor (p75NTR) extracellular domain, comprising a variant amino acid at one or more of position 75, 109, 133 and/or 134 within said p75NTR, fusion protein or pharmaceutical composition of the invention is for use in a human, canine, feline equine, bovine, or cameline. In one or more embodiments, the p75NTR protein or portion thereof, fusion protein or pharmaceutical composition of the invention is for use in a canine. In one or more embodiments, the p75NTR protein or portion thereof, fusion protein or pharmaceutical composition of the invention is for use in a feline. In one or more embodiments, the variant p75NTR protein or portion thereof, fusion protein or pharmaceutical composition of the invention is for use in an equine. In one or more embodiments, the p75NTR protein or portion thereof, fusion protein or pharmaceutical composition of the invention is for use in a bovine. In one or more embodiments, the p75NTR protein or portion thereof, fusion protein or pharmaceutical composition of the invention is for use in a cameline. In one or more embodiments, the p75NTR protein or portion thereof, fusion protein or pharmaceutical composition of the invention is for use a human.

In one embodiment, the isolated polypeptide comprising a p75 neurotrophin receptor (p75NTR) extracellular domain, comprising a variant amino acid at one or more of position 75, 109, 133 and/or 134 within said p75NTR,, fusion protein or pharmaceutical composition of the invention is administered together with one or more therapeutic agent, for example a therapeutic agent to treat pain.

The p75NTR proteins of the invention are optionally administered in combination with one or more active agents including other analgesic agents. Such active agents include analgesic, anti-histamine, antipyretic, anti-inflammatory, antibiotic, antiviral, and anti-cytokine agents. Active agents include agonists, antagonists, and modulators of TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include, without limitation, 2-arylpropionic acids, aceclofenac, acemetacin, acetylsalicylic acid (Aspirin), alclofenac, alminoprofen, amoxiprin, ampyrone, arylalkanoic acids, azapropazone, benorylate/benorilate, benoxaprofen, bromfenac, carprofen, celecoxib, choline magnesium salicylate, clofezone, COX-2 inhibitors, dexibuprofen, dexketoprofen, diclofenac, diflunisal, droxicam, ethenzamide, etodolac, etoricoxib, faislamine, fenamic acids, fenbufen, fenoprofen, flufenamic acid, flunoxaprofen, flurbiprofen, ibuprofen, ibuproxam, indometacin, indoprofen, kebuzone, ketoprofen, ketorolac, lomoxicam, loxoprofen, lumiracoxib, magnesium salicylate, meclofenamic acid, mefenamic acid, meloxicam, metamizole, methyl salicylate, mofebutazone, nabumetone, naproxen, n-arylanthranilic acids, oxametacin, oxaprozin, oxicams, oxyphenbutazone, parecoxib, phenazone, phenylbutazone, phenylbutazone, piroxicam, pirprofen, profens, proglumetacin, pyrazolidine derivatives, rofecoxib, salicyl salicylate, salicylamide, salicylates, sulfinpyrazone, sulindac, suprofen, tenoxicam, tiaprofenic acid, tolfenamic acid, tolmetin, and valdecoxib.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Antibiotics include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, meticillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilimide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclometasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

The most common form of current treatment for OA and pain related to OA is NSAIDs (which are also anti-pain medications). NSAIDs are not always sufficiently effective, typically need to be administered daily and none are approved for long-term use in cats in the US. Additionally, there are safety and tolerability concerns with the use of NSAIDS in both dogs and cats, especially with long-term treatment. NSAIDs are not recommended to be co-administered with anti-NGF mAbs for long periods.

In certain embodiments, treatment comprises coadministration of dietary supplements containing Omega-3 fatty acids, microlactin, and/or glucosamine/chondroitin as an aid to joint health. Adequan (polysulfated glycosaminoglycan) is an FDA-approved disease modifying drug that inhibits cartilage loss and may also be co-administered.

The isolated polypeptide comprising a p75 neurotrophin receptor (p75NTR) extracellular domain, comprising a variant amino acid at one or more of position 75, 109, 133 and/or 134 within said p75NTR fusion protein or pharmaceutical composition may be administered at the same time or at a different time as the other therapy or therapeutic compound or therapy, e.g., simultaneously, separately or sequentially.

The invention also provides an in vitro, ex vivo or in vivo method for inhibiting NGF activity in a subject comprising administering the isolated polypeptide comprising a p75 neurotrophin receptor (p75NTR) extracellular domain, comprising a variant amino acid at one or more of position 75, 109, 133 and/or 134 within said p75NTR, fusion protein or pharmaceutical composition of the invention.

In one or more aspects, the present invention provides a method of producing the fusion protein of the invention by culturing the host cell of the invention under conditions that result in production of the fusion protein and subsequently isolating the fusion protein from the host cell or culture medium of the host cell.

In another aspect, the invention provides a kit for the treatment or prevention of a disease, diagnosis, prognosis or monitoring disease comprising the isolated polypeptide comprising a p75 neurotrophin receptor (p75NTR) extracellular domain comprising a variant amino acid at one or more of positions 75, 109, 133 and/or 134 within said p75NTR, fusion protein or pharmaceutical composition of the invention. Such a kit may contain other components, packaging and/or instructions.

The invention in another aspect provides an isolated polypeptide comprising a p75 neurotrophin receptor (p75NTR) extracellular domain, comprising a variant amino acid at one or more of position 75, 109, 133 and/or 134 within said p75NTR, fusion protein or pharmaceutical composition of the invention packaged in lyophilized form or packaged in an aqueous medium.

In another aspect, isolated polypeptide comprising a p75 neurotrophin receptor (p75NTR) extracellular domain, comprising a variant amino acid at one or more of position 75, 109, 133 and/or 134 within said p75NTR, fusion protein or pharmaceutical composition of the invention as described herein is used for non-therapeutic purposes, such as diagnostic tests and assays. Thus, the present invention also provides the above p75NTR proteins and fusion proteins for use in diagnostic methods for detecting NGF in a subject, particularly a human, canine or feline subject but not limited thereto, known to be or suspected of having an NGF related disorder. Methods for detecting NGF in a subject known to be or suspected of having an NGF related disorder may include exposing a sample from the subject to a labelled protein of the invention and detecting said labelled protein. A diagnostic method may be used to quantitatively or qualitatively detect the NGF in a sample or to detect presence of cells that express the NGF.

Further aspects and embodiments of the invention will be apparent to those skilled in the art given the present disclosure including the following experimental exemplification.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including any references to gene accession numbers and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### Numbered Embodiments

1. An isolated polypeptide comprising a companion animal p75 neurotrophin receptor (p75NTR) extracellular domain, wherein said p75NTR comprises a variant amino acid at one or more of position 75, 109, 133 and/or 134.
2. The isolated polypeptide according to embodiment 1, wherein the variant amino acid at position 75 of p75NTR comprises a polar side chain.
3. The isolated polypeptide according to any preceding embodiment, wherein the variant amino acid at position 75 of p75NTR is selected from selected from serine, threonine, tyrosine, tryptophan, asparagine, glutamine or cysteine, preferably threonine.
4. The isolated polypeptide according to any preceding embodiment, wherein the variant amino acid at position 109 of p75NTR comprises an aromatic side chain.
5. The isolated polypeptide according to any preceding embodiment, wherein the variant amino acid at position 109 of p75NTR is selected from histidine, tyrosine, phenylalanine, or tryptophan.
6. The isolated polypeptide according to any preceding embodiment, wherein the variant amino acid at position 133 of p75NTR comprises a charged side chain.
7. The isolated polypeptide according to any preceding embodiment, wherein the variant amino acid at position 133 of p75NTR is selected from arginine, histidine, lysine, aspartic acid, or glutamic acid.
8. The isolated polypeptide according to any of embodiments 1 to 6, wherein the variant amino acid at position 133 of p75NTR comprises a negatively charged side chain.
9. The isolated polypeptide according to embodiment 8, wherein the variant amino acid at position 133 of p75NTR is selected from arginine, histidine, or lysine, preferably arginine.
10. The isolated polypeptide according to any preceding embodiment, wherein the variant amino acid at position 134 of p75NTR comprises a hydrophobic side chain.
11. The isolated polypeptide according to any preceding embodiment, wherein the variant amino acid at position 134 of p75NTR is selected from alanine, valine, isoleucine, leucine, methionine, phenylalanine, tyrosine, or tryptophan.
12. The isolated polypeptide according to any of embodiments 1 to 10, wherein the variant amino acid at position 134 of p75NTR comprises a non-aromatic hydrophobic side chain.
13. The isolated polypeptide according to embodiment 12, wherein the variant amino acid at position 134 of p75NTR is selected from alanine, valine, isoleucine, leucine, or methionine, preferably leucine.
14. The isolated polypeptide according to any preceding embodiment, wherein the companion animal is a cat, dog, pig, cow, horse, or camel.
15. The isolated polypeptide according to any preceding embodiment, wherein the companion animal is a dog and the p75NTR comprises or consists of a sequence selected from SEQ ID NO:46, SEQ ID NO:49, SEQ ID NO:52, SEQ ID NO:55 or SEQ ID NO:58.
16. The isolated polypeptide according any of embodiments 1 to 14, wherein the companion animal is a cat and the p75NTR comprises or consists of a sequence selected from SEQ ID NO:61, SEQ ID NO:63, SEQ ID NO:65, SEQ ID NO:67 or SEQ ID NO:69.
17. The isolated polypeptide according to a preceding embodiment wherein the p75NTR extracellular domain is truncated.
18. An isolated nucleic acid encoding the isolated polypeptide according to a preceding embodiment.
19. A vector comprising a nucleic acid according to embodiment 18.
20. A host cell comprising a nucleic acid according to embodiment 18 or a vector according to embodiment 19.
21. A fusion protein comprising a companion animal p75NTRextracellular domain, wherein said p75NTR comprises a variant amino acid at one or more of position 75, 109, 133 and/or 134, and a half-life extending moiety.
22. The fusion protein according to embodiment 21 wherein the half-life extending moiety is selected from an Fc domain, a serum albumin binder or PEG.
23. The fusion protein according to embodiment 21 wherein the half-life extending moiety is a wild type or mutant Fc domain.
24. The fusion protein according to any of embodiments 21 to 23 wherein the half-life extending moiety is an Fc domain and the p75NTR extracellular domain or portion thereof and the Fc domain are linked with a linker.
25. The fusion protein according to embodiment 24 wherein the linker is a peptide linker.
26. The fusion protein according to embodiment 25 wherein the peptide linker is (G₄S)ₙ wherein n is 1 to 4.
27. The fusion protein according to any of embodiments 21 to 26 wherein the companion animal is a cat, dog, pig, cow, horse, or camel.
28. The fusion protein according to any of embodiments 21 to 27 wherein the Fc domain is a canine Fc domain.
29. The fusion protein according to embodiment 28 wherein the fusion protein comprises or consists of a sequence selected from SEQ ID NO:47, SEQ ID NO:50 SEQ ID NO:53, SEQ ID NO:56 or SEQ ID NO:59.
30. The fusion protein according to any of embodiments 21 to 27 wherein the Fc domain is a feline Fc domain.
31. The fusion protein according to embodiment 30 wherein the fusion protein comprises or consists of a sequence selected from SEQ ID NO:62, SEQ ID NO:64, SEQ ID NO:66, SEQ ID NO:68 or SEQ ID NO:70.
32. A nucleic acid encoding a fusion protein according to any of embodiments 21 to 31
33. A vector comprising a nucleic acid according to embodiment 32.
34. A host cell comprising a nucleic acid according to embodiment 32 or a vector according to embodiment 33.
35. A pharmaceutical composition comprising an isolated polypeptide according to any of embodiments 1 to 17, or a fusion protein according to any of embodiments 21 to 31.
36. A method for treating an NGF-related disorder in a companion animal comprising administering an isolated companion animal p75NTR protein according to any of embodiments 1 to 17, a fusion protein according to any of embodiments 21 to 31 or a pharmaceutical composition of embodiment 35.
37. The use of an isolated companion animal p75NTR protein according to any of embodiments 1 to 17, a fusion protein according to any of embodiments 21 to 31 or a pharmaceutical composition of embodiment 35 in the treatment of an NGF-related disorder in a companion animal.
38. An isolated companion animal p75NTR protein according to any of embodiments 1 to 17, or a fusion protein according to any of embodiments 21 to 31 for use in the treatment of an NGF-related disorder in a companion animal.
39. The method of embodiment 36 or the use of embodiment 37 or the isolated companion animal p75NTR protein or a fusion protein for use according to embodiment 38 wherein the NGF-related disorder is cardiovascular diseases, atherosclerosis, obesity, type 2 diabetes, metabolic syndrome, pain and inflammation.
40. The method or use of embodiment 39 wherein the NGF-related disorder is a pain related disorder.
41. The method or use of embodiment 40 wherein pain is selected from osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, and gastrointestinal pain.
42. A method of inhibiting NGF activity in a companion animal comprising administering an isolated companion animal p75NTR protein according to any of embodiments 1 to 17, a fusion protein according to any of embodiments 21 to 31 or a pharmaceutical composition of embodiment 35.
43. The method or use of embodiment 40 or embodiment 41 or the method of embodiment 42 comprising administration of a second compound that treats pain.
44. A kit comprising an isolated companion animal p75NTR protein according to any of embodiments 1 to 17, a fusion protein according to any of embodiments 21 to 31 or a pharmaceutical composition of embodiment 35 and optionally instructions for use.

The invention is further described in the non-limiting examples.

### EXAMPLES

### Example 1—Structural Analyses of p75NTR-NGF Interaction.

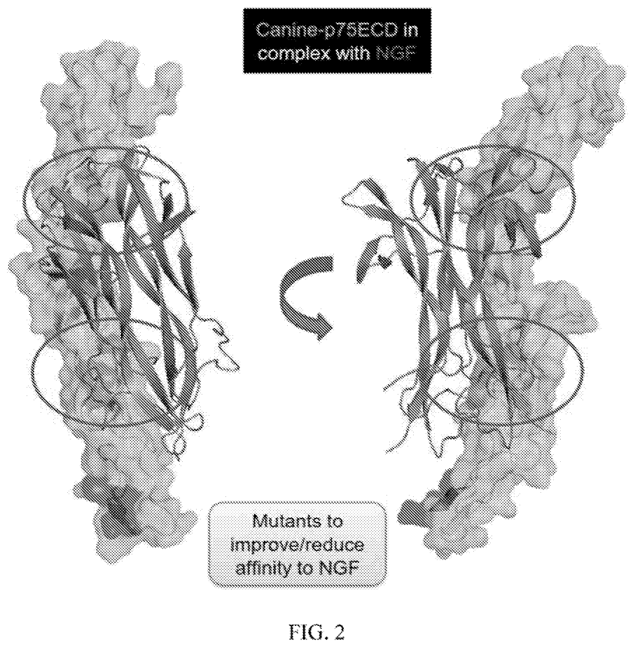

A model of p75NTR extracellular domain as well as NGF was generated using alpha fold server (human crystal structure used as template—pdb code 3BUK) (FIG. 2). Interacting residues between p75NTR and NGF were identified using distance and geometry cut-off. Mutations to improve/reduce binding as well as improve expression were identified.

FIG. 1 demonstrates alignment of canine, feline, bovine and equine p75NTR sequences which show high sequence identity between said species.
Canine vs Feline: 1/164 (more than 99% identical)
Canine vs Equine: 4/164 (97.6% identical)
Feline vs Equine: 5/164 (97% identical)
Canine vs Bovine: 2/164 (98.8% identical)

### Example 2—Protein Constructs and CHO-s Transfection/Expression

The amino acid sequences for PetML119 variants are given in the Sequences section below.

For protein production, DNA constructs were generated to encode chimeric Fc fusion protein comprising selected canine IgG constant regions (between hinge and C-terminus) fused to the extracellular domain of canine p75 lacking predicted O-glycosylation and γ-secretase sites.

Both the canine IgG-B Fc domain and the p75 extracellular domain (res 31-194 from UniProtKB—J9PAM0) were synthesised. Both genes were PCR amplified using Q5 high fidelity DNA polymerase (using specific primers including overlapping regions to allow assembly) and assembled into mammalian expression vector PetML119var using NEBuilder HIFI DNA Assembly (New England Biolabs). In the expression vector, the fusion protein chain and the antibiotic resistant gene expression units are flanked by DNA transposon piggyBac terminal inverted repeats to mediate stable integration into host cells in the presence of piggyBac transposase. The expression vector was then transfected into a suitable mammalian cell line such as CHO cells together with PiggyBac transposase to obtain stable expression. For fusion protein production, 1×10⁶/mL selected CHO cells are seeded in 800 mL culture media (F17+4 mM 1-Gln+0.3% P188+1:500 ACA) and incubated at 32° C., 8% CO₂ with shaking at 130 rpm. 2% HyClone Cell Boost 7a supplement+0.2% HyClone Cell Boost 7b supplement 2 mM glucose is added to the media daily from the 4^{th} day of overproduction. Culture supernatants are collected on day 10 and the protein concentration is determined using surface plasmon resonance using protein A chip (Biacore 8K, Cytiva Life Sciences).

Typically, PetML119var showed peak of expression at 10 day in production reaching between 20 to 150 mg/L, with high affinity molecules showing a 2× improvement in respect to WT molecule (SEQ ID NO:11), while low affinity showed slightly lower titres.
PetML119: 60 mg/L
PetML119-E75T: 20 mg/L
PetML119-S109Y: 130 mg/L
PetML119-S109H: 150 mg/L
PetML119-V133R: 120 mg/L
PetML119-D134L: 30 mg/L

### Example 3—PetML119var Purification

Cell suspensions from PetML119var stable transfected clones, cultured as described for at least 7 days, were filtered using 0.22 um filters after being incubated for 10 minutes with Sartoclear Dynamics® Lab V (SDLV-0500-20C—E). Cleared supernatants were loaded into Mabselect sure LX prepacked 20 mL column (17547402), pre-equilibrated with PBS. The column was washed with 40 mLs of PBS (2CV) and then fusion proteins have been eluted using gradient (0-100% in 2CV) of 0.1M Glycine pH2.7. Fractions containing fusion proteins were pooled together and neutralised with 100 mM TRIS pH8 (final concentration).

Neutralised fusion protein pooled fractions were concentrated to 5 mL and loaded into PBS pre-equilibrated HiLoad 16/600 Superdex 200 pg (28989335) as second step purification. Monomeric fractions (based on previously analysed protein standards' retention times) were pooled and protein concentration was assessed using NanoDrop™ One (Thermo Scientific™).

Around 10-60 mg/L of purified product was obtained following the above mentioned protocol. Variants were purified to the same extent as WT; yield of the variants were not altered during the purification process relative to WT.
PetML119: 30 mg/L after 2 steps purification
PetML119-E75T: 10 mg/L after 2 steps purification
PetML119-S109Y: 60 mg/L after 2 steps purification
PetML119-S109H: 63.3 mg/L after 2 steps purification
PetML119-V133R: 56.6 mg/L after 2 steps purification
PetML119-D134L: 13.3 mg/L after 2 steps purification

### Example 4—HPLC Analytical Chromatography

Purified material purity was assessed using both Size Exclusion Chromatography (SEC), for oligomerisation analyses, and cation exchange chromatography (SCX) for charge variants analyses.

HPLC-SEC chromatography (column: BioResolve SEC mAb 200A, 2.5 um column WATERS) was performed using ACQUITY H-class Bio from WATERS using PBS as mobile phase with isocratic 0.575 mL/min flow rate.

HPLC-SCX chromatography (column: BioResolve SCX mAb Column, 3 μm, 4.6 mm×100 mm) was performed using ACQUITY H-class Bio from WATERS using MES pH5 as mobile phase with salt gradient used to separate charge variants at 0.9 mL/min flow rate.

10 uL of each sample was injected into both H-SEC and H-SCX using the above-mentioned protocol. Percentage of monomeric species and Area (indicative of protein concentration) were determined for each molecule.

Both PetML119wt and var showed very high purity (more than 99%) by HSEC and few charge variants (potentially corresponding to different glycoforms) were observed by HSCX, confirming that variants of p75 do not impact final purity and homogeneity of the product.

### Example 5—Protein A Binding Affinity Validation

Purified fusion proteins in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to Protein A was assessed using Biacore 8K (Cytiva).

Briefly, Sensor Chip Protein A (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

Fusion protein dilutions were prepared diluting PetML 119var from 1 uM to 4 nM (6 concentrations with 1:3 dilutions) in Running Buffer and kinetics was assessed using single cycle kinetics method (Biacore Assay Handbook, Cytiva). Kinetics and/or Affinity quantification have been performed using Biacore Insight following standard analyses methods.

The results show that none of p75NTR mutations induced changes in protA binding.

### Example 6—Human and Rat Nerve Growth Factor (h-rNGF) Binding Affinity Determination

Purified fusion proteins in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to human and rat NGF was assessed using Biacore 8K (Cytiva). Briefly, Protein A Sensor Chip (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

PetML 119var was diluted into running buffer at 6 nM concentration. These were immobilised using 90 sec association at 10 uL/min as capturing step, followed by injection of running buffer to remove any unbound product.

Human and rat NGF (from Bio-Techne Ltd—556-NG/CF/256-GF-100/CF) was diluted in Running Buffer at 100 nM with 1:2 further dilutions down to 4.68 nM. Kinetics were assessed using multi-cycle kinetics with capture step method (30 sec association—300 sec dissociation) followed by regeneration step (0.1M Glycine pH2.2 contact time 60 sec FR 30 uL/min). Kinetics quantification have been performed using Biacore Insight following standard analyses methods.

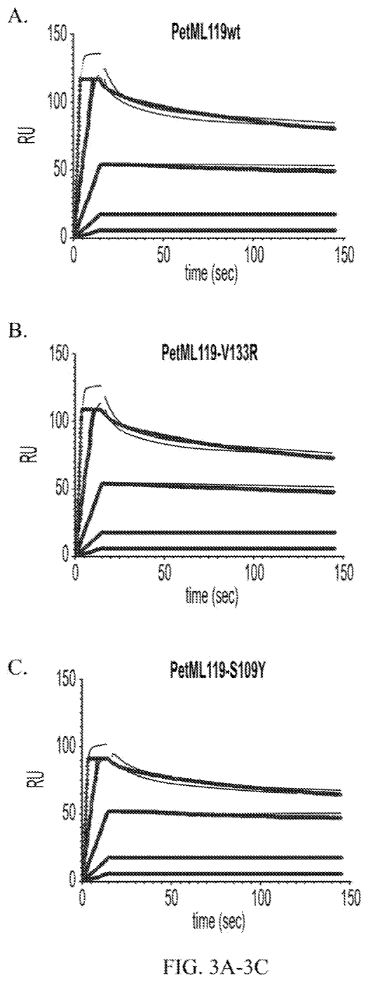

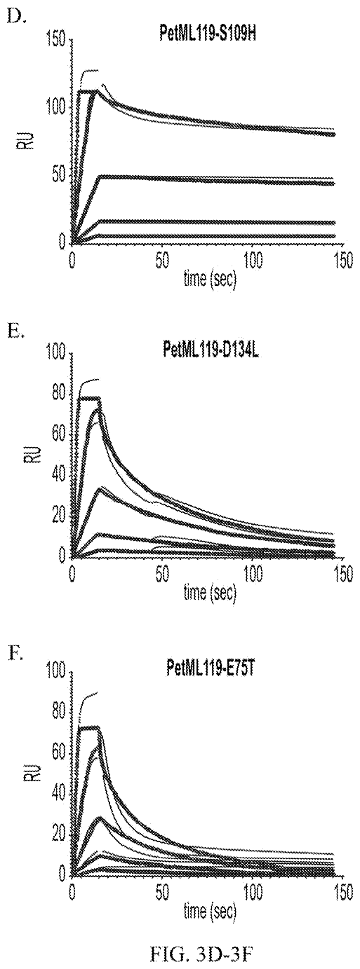

Results showed subnanomolar KD for both human and rat NGF with PetML119wt improved variants (FIG. 3 and Table 1). Low affinity mutants to NGF showed 20× and 40× reduced affinity relative to that observed with PetML119wt. Both PetML119wt and variants displayed dissociation from NGF over time, indicating that the binding is reversible. This contrasts with that observed with anti-NGF mAb (Bedinvetmab- anti-NGF IgG control, unrelated fusion protein and RPMI without NGF as negative control WHO Drug Information, Vol. 32, No. 4, 2018-pg568)) where the binding remained constant over time.

TABLE 1
Binding affinity using hNGF

| Antibody/Fc-protein | KD (nM) | RMAX |
|---|---|---|
| Ant-NGF mAb | 0.0073 | 837.1 |
| PetML119 | 0.309 | 116.7 |
| PetML119-E75T | 12.4 | 77.1 |
| PetML119-S109Y | 0.225 | 91.1 |
| PetML119-S109H | 0.267 | 111.6 |
| PetML119-V133R | 0.346 | 109.2 |
| PetML119-D134L | 6.8 | 80.9 |

### Example 7—Unfolding and Oligomerisation Determination

Purified fusion proteins in PBS were concentrated using centrifugal concentrators (Sartorious—VS02H22) to 5 mg/mL Protein concentration was assessed using UV absorbance at 280 nm with NanoDrop™ One (Thermo Scientific™).

Tm and Tagg analyses were performed on UnCle from Unchained labs using a standard protocol.

Briefly, 10 uL of fusion protein was used to determine unfolding and aggregation events during a temperature ramp (from 25° C. till 95° C.).

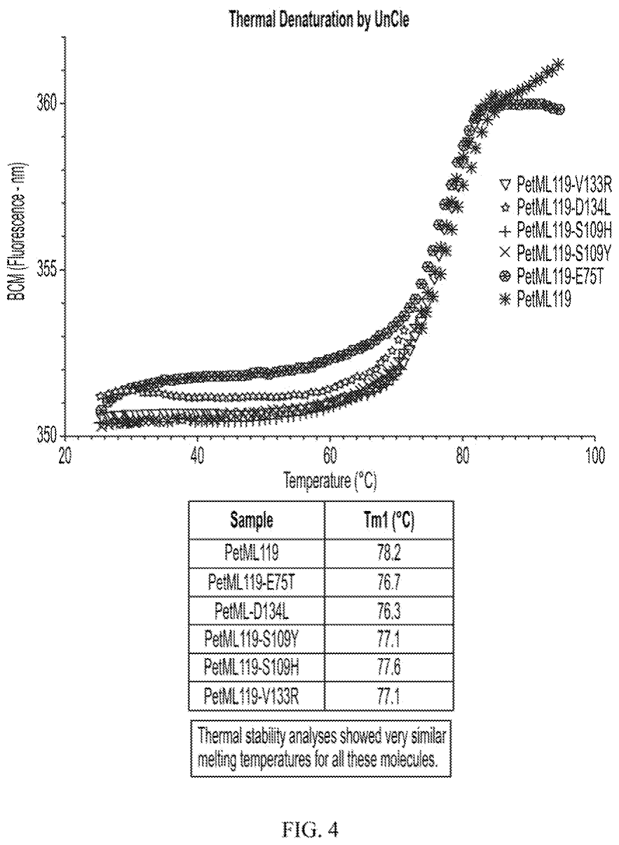

As anticipated from previous results, PetML119 showed no aggregation up to 95° C. with Tm1 around 78° C. Similarly, all mutants showed no aggregation up to 95° C. with Tm1 around 76° C. (FIG. 4).

### Example 8—In vitro NGF Inhibition Assay

To assess biological activity of the p75 fusion protein, an NGF-dependent cell line was used. Proficient sequestration of NGF by our fusion protein will result in slower proliferation in comparison to control.

TF-1 cell line was bought from ATCC (CRL-2003) and kept in culture using standard aseptic methods using complete RPMI (10% FBS+2 mM 1-Gln+10 ng/ml hNGF).

2 million TF-1 cells were labelled with 2.5 uM CFSE cell trace (Invitrogen—C34554) in 1 mL of RPMI only for 30 minutes at RT in the dark. Cells were then washed 2× in complete RPMI media, counted again and seeded at 10000 cells/mL (1 mL total volume per well) in 24-well plate.

Dilution of PetML119wt and variants or controls (Bedinvetmab—anti-NGF IgG control, RPMI without NGF as negative control) were diluted in 100 uL of RPMI media from 3 uM concentration till 91.25 nM. 100 uL of protein dilution was added to each well.

Plates were analysed after 3 days. Briefly, 1 mL cell suspension was centrifuged for 5 minutes at 300 g RT, washed 2 times with FACS buffer (PBS+3% FBS+3 mM EDTA) and finally resuspended in 100 uL of FACS buffer. Cells were acquired using CytoFLEX Flow Cytometer using following parameters (FSC:20; SSC:50; FITC:1; threshold:1313131). Cells were gated based on FITC fluorescence (more fluorescence less proliferation) and % of proliferation inhibition was calculated considering 100% inhibition TF-1 cells cultured in RPMI without NGF and 0% inhibition cells cultured with complete RPMI media. Graph pad was used to calculate IC50 values.

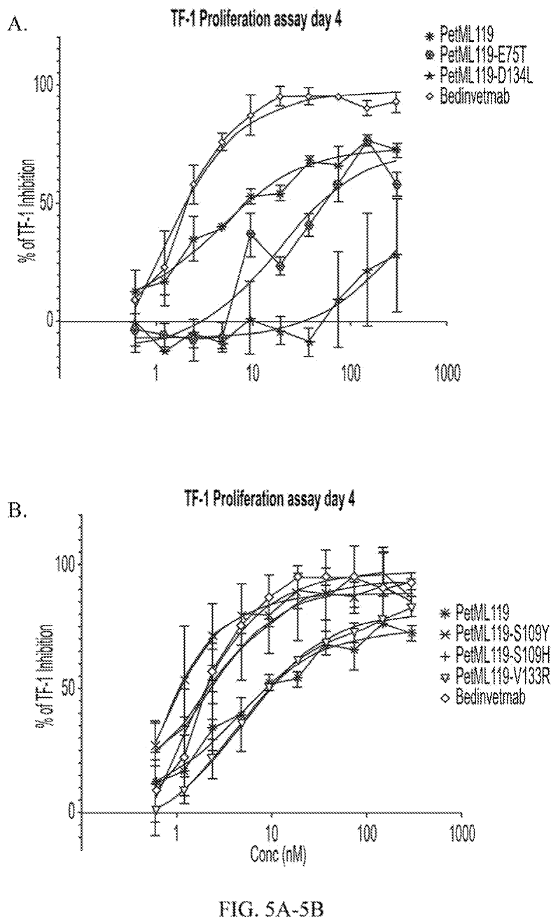

PetML 119wt, variants and Bedinvetmab were able to inhibit TF-1 proliferation in a dose dependent manner (FIG. 5, Table 2). Incubation with Bedinvetmab showed complete inhibition, whilst incubation with PetML119wt had an IC MAX around 60-70%. Low affinity NGF binding molecules showed a reduced inhibition ability (particularly D134L mutant) relative to PetML119wt. High affinity NGF binding molecules showed an improved inhibition ability relative to PetML 119wt, similar to Bedinvetmab. Importantly, the IC MAX of all variants did not reach 100, indicating partial inhibition. This contrasts with anti-NGF Bedinvetmab which shows an IC Max of 100, indicating complete inhibition.

TABLE 2

| | | PetML119- | PetML119- | |
|---|---|---|---|---|
| | PetML119 | E75T | D134L | Bedinvetmab |
| IC50 (nM) | 4.455 | 18.16 | 328.8 | 0.8899 |
| ICMAX (%) | 73.1 | 72.51 | 70.54 | 96.83 |
| | | PetML119- | PetML119- | PetML119- |
| | PetML119 | S109H | S109Y | V133R |
| IC50 (nM) | 4.455 | 0.1701 | 2.146 | 5.061 |
| ICMAX (%) | 73.1 | 88.51 | 93.45 | 80.49 |

### Example 9—PathHunter® eXpress Receptor Tyrosine Kinase Functional Assay Kit

To further prove that our molecules are able to inhibit NGF pathway activation in-vitro, the PathHunter® eXpress Receptor Tyrosine Kinase Functional Assay Kit was used. This included a cell line engineered to have TrkA intracellular domain fused with a small complementing fragment of β-Gal.

The larger portion of β-gal, termed EA for ‘enzyme acceptor’, was fused to proteins containing phospho-tyrosine binding domains. Ligand-induced activation of the receptor causes either homo or hetero-dimerization of the receptor which results in cross-phosphorylation. The SH2-EA fusion protein then specifically binds the phosphorylated receptor resulting in complementation of the two fragments of β-gal and formation of a functional enzyme. β-gal activity is then quantitatively detected using the chemiluminescent substrate in the PathHunter Detection Kit. Briefly, 3 uM concentration till 91.25 nM of PetML119wt, var and positive control anti-NGF mAB (Bedinvetmab) was used while NGF was used at 2 nM.

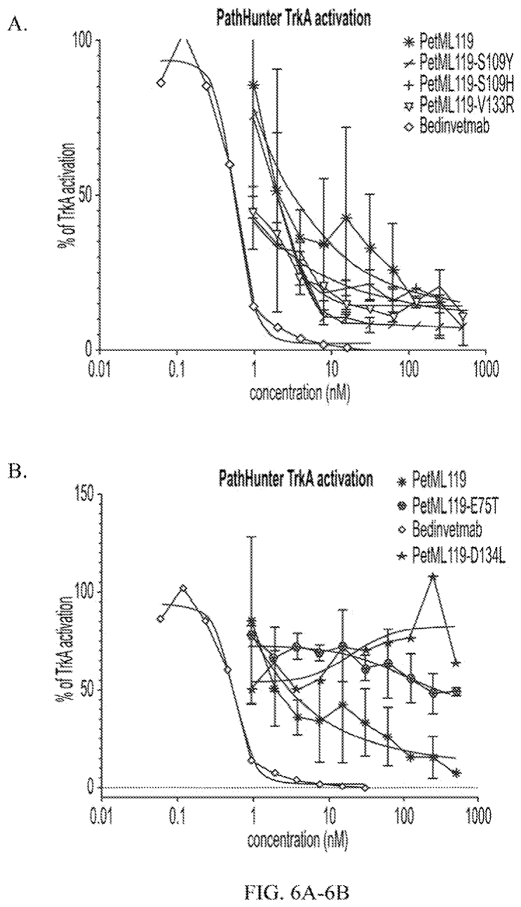

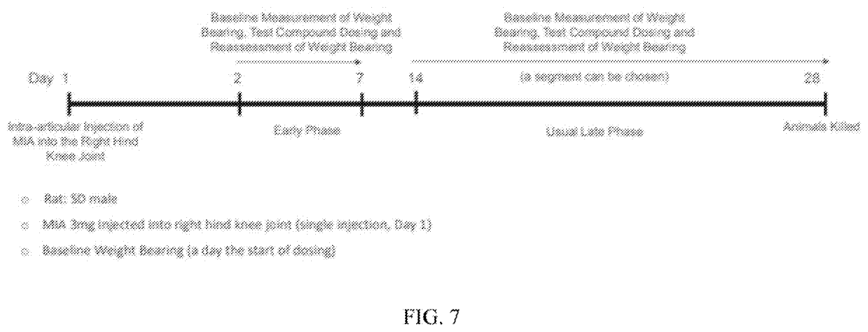

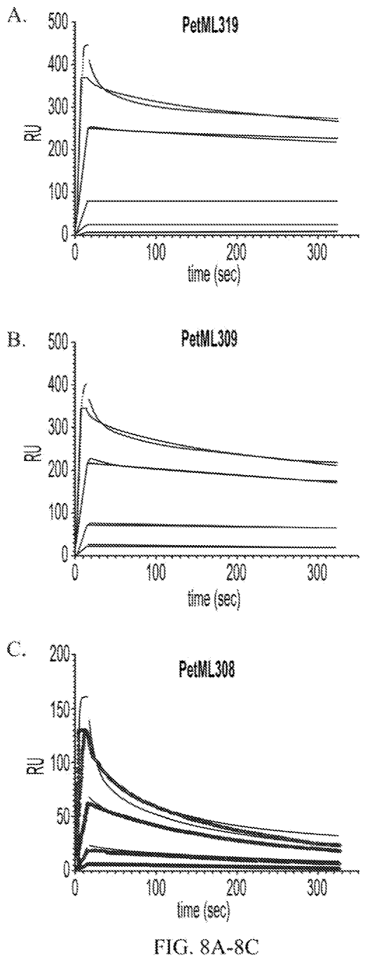

Luminescence readings showed that, similarly to that seen in the TF-1 assay, anti-NGF mAb has a distinct sigmoidal shape inhibition curve reaching 100% TrkA inhibition (FIG. 6). In contrast to anti-NGF but consistent with the TF-1 assay, PetML119 molecules showed shallower inhibition curve that did not reach 100% inhibition. Also, high affinity NGF PetML119 molecules showed increased max inhibition in comparison to WT (although still not reaching 100% inhibition) as well as reduced IC50, whilst low affinity NGF PetML119 molecules showed reduced max inhibition in comparison to WT as well as increased IC50, again consistent with the TF-1 inhibition assay (Table 3).

Altogether these results suggest that anti-NGF mAb Bedinvetmab differs from PetML119 molecules both on inhibition concentration and amplitude of inhibition. Our new molecules of rationally engineered PetML119 mutants can either approach Bedinvetmab level NGF inhibition or further reduce PetML119wt NGF inhibition. Importantly, this is achieved without achieving 100% inhibition of NGF, which provides a mechanistic basis behind its expected DMOAD activity in-vivo.

TABLE 3

| | | PetML119- | PetML119- | PetML119- | PetML119- | PetML119- | |
|---|---|---|---|---|---|---|---|
| | PetML119 | S109Y | S109H | V133R | E75T | D134L | Bedinvetmab |
| Best-fit | | | | | | | |
| Bottom (%) | 9.5 | 7.794 | 10.35 | 14.33 | 44.45 | 53.68 | 5.352 |
| Top (%) | Unstable | 87.3 | 48.3 | 49.29 | 72.63 | 82.73 | 123 |
| IC50 (nM) | 8.04 | 2.154 | 2.201 | 2.56 | 82.62 | 20.84 | 4.238 |

### Example 10—MIA-induced OA in Rats (Efficacy and pK)

Efficacy and half-life for PetML119/122 were analysed using a rat model of OA. The detailed protocol is shown below. The model is shown in FIG. 6.

### Induction of Arthritis

Osteoarthritis was induced chemically by an intra-articular (I.A.) injection of 3 mg of monosodium-iodoacetate (MIA) (in 25 μL saline) into the right hind limb knee joint of the rat given under isoflurane anesthesia. While under anesthesia, ophthalmic ointment was applied to both eyes. The day of I.A. injection of MIA was counted as Day 0.

### Allocation to Treatment Groups

Baseline dynamic weight bearing (DWB) were measured for all rats. Body weight (BW) was also measured at the same time. Rats were anesthetized and MIA injected into the right Knee joint through the middle of the patellar tendon approximately perpendicular to the tibia (Intra-articular (I.A.)). Dose level for I.A. injection of MIA was selected based on previous literature report in rodents (Bove et al.: Weight bearing as a measure of disease progression and efficacy of anti-inflammatory compounds in a model of monosodium iodoacetate-induced osteoarthritis. Osteoarthritis Cartilage. 2003 Nov; 11(11):821-830). Animals showing a significant weight bearing difference between the MIA injected limb (right) and the healthy limb (left) were assigned to the study. Randomization was done based on both baseline DWB and BW (two variables randomization).

### Dynamic Weight Bearing (DWB) Evaluation

Dynamic Weight Bearing was evaluated using the BioSeb® automated DWB system according to the manufacturer's manual. A two-minute recording was done for each rat. Analysis of dynamic weight bearing data was done off-line using the BioSeb® software. The system automatically calculated the weight borne by each limb and the tail. Body weight was measured for each rat immediately before the DWB for each time of testing. DWB measurement was done at different time points as per schedule in Study Design. Total distance travelled was also noted during DWB data analysis.

Dosing with Test Items

Group 1-2 rats received intravenous (IV) injections of vehicle and Group 3-7 rats received IV injections with the test items at designated doses once on Day 3 as depicted in the table below. Group 8 rats received oral gavage dosing with dexamethasone once daily from Day 3-21 as depicted in the table below.

### Monosodium-Iodoacetate (MIA)-Induced Rat Study

Study design

TABLE 4

| | | TI Dose | TI Route, | TI Dosing | | |
|---|---|---|---|---|---|---|
| *Gr | Group Treatment | level | Volume | schedule | DWB testing | N |
| 1 | Naive/ Vehicle PBS | 0 | IV, 5 mL/kg | Day 3, | Day 1 (baseline), | 10 |
| | | | | QD | Days 3, 6, 14, 21 | |
| 2 | 3 mg MIA/ Vehicle | 0 | IV, 5 mL/kg | Day 3, | Day 1 (baseline), | 10 |
| | PBS | | | QD | Days 3, 6, 14, 21 | |
| 3 | 3 mg MIA/ Sample 1 | 0.5 mg/kg | IV, 5 mL/kg | Day 3, | Day 1 (baseline), | 10 |
| | (PetML119) | | | QD | Days 3, 6, 14, 21 | |
| 4 | 3 mg MIA/ Sample 2 | 0.5 mg/kg | IV, 5 mL/kg | Day 3, | Day 1 (baseline), | 10 |
| | (PetML119-E75T) | | | QD | Days 3, 6, 14, 21 | |
| 5 | 3 mg MIA/ Sample 3 | 0.5 mg/kg | IV, 5 mL/kg | Day 3, | Day 1 (baseline), | 10 |
| | (PetML119-S109Y) | | | QD | Days 3, 6, 14, 21 | |
| 6 | 3 mg MIA/ Sample 1 | 0.1 mg/kg | IV, 5 mL/kg | Day 3, | Day 1 (baseline), | 10 |
| | (PetML119) | | | QD | Days 3, 6, 14, 21 | |
| 7 | 3 mg MIA/ Sample 2 | 0.1 mg/kg | IV, 5 mL/kg | Day 3, | Day 1 (baseline), | 10 |
| | (PetML119-E75T) | | | QD | Days 3, 6, 14, 21 | |
| 8 | 3 mg MIA/ Sample 3 | 0.1 mg/kg | IV, 5 mL/kg | Day 3, | Day 1 (baseline), | 10 |
| | (PetML119-S109Y) | | | QD | Days 3, 6, 14, 21 | |

*Animals from Groups 2 to 8 plus spares receive a single intra-articular (IA) injection with MIA (3 mg/25 μL saline) and the day of MIA injection is considered as Day 0 (in the right knee joint).
IV: Intravenous (tail vein);
PO: oral gavage;
QD—once daily

Treated animals were observed for any clinical signs during the study. DWB was analyzed on Days 3, 6, 14 and 21. Joint diameter was measured using a caliper on the right knee joint (medio-laterally) on days 3, 6, 14 and 21.

PK bleeds from 5 rats per group were taken using standard procedures on alternative days.

### Serum pk Analysis

Sandwich ELISA to quantify serum levels of our fusion protein were set up as follows:
30 uL of 2 ug/mL of Capturing antibody (Mouse anti-canine p75 Ab -->MAB367-SP (Novus Bio)) diluted in PBS +0.1M sodium bicarbonate were immobilised on half-area ELISA plates (MICROPLATE, 96 WELL, PS, HALF AREA, CLEAR, Item No.: 675061) overnight at 4° C.
Plates were washed 2× with 200 uL blocking solution (PBS+5% DNFM+0.2% Tween20) and blocking have been performed with 150 uL of blocking solution across all wells for 3 hrs at RT.
Sera from different timepoints/groups of rat study were diluted 100× in blocking solution (2 uL serum+198 uL blocking solution) and 30 uL were added to relevant wells. Standards from PetML119 were prepared diluting fusion protein into rat serum from 100 ug/mL till Ing/mL with 1:5 dilutions. Standards were then diluted 100× in blocking solution and 30 uL have been added to relevant wells.
Sera were incubated for 1 hr at RT with 450 rpm shaking.
Plates were washed 2× with 200 uL blocking solution; detection antibody (SA5-10309 (ThermoFisher)) have been diluted 1:40000 in blocking solution and 30 uL were added to each well and left 30′ at RT with 450 rpm shaking.
Plates were washed 2× with 200 uL blocking solution; developing HRP-conjugated antibody (A16035 (ThermoFisher)) were diluted 1:10000 in blocking solution and 30 uL were added to each well and left 30′ at RT with 450 rpm shaking.
Plates were washed 2× with 200 uL blocking solution then 2× with 200 uL of PBS+0.2% Tween20 and finally 50 uL of TMB (TMB Chromogen Solution (for ELISA) -->002023) were added to each well. After 10′, when standard curve showed saturation in first two points, the reaction was stopped adding 50 uL of 1M Sulphuric acid.
All wells were read with CLARIOstar Plus (BMG LABTECH) using endpoint Absorbance at 650 nm and 450 nm. Values were imported in Graph Pad and one-phase decay fitting have been applied to estimate half-life of these.

### Example 11—Disease-Modifying Osteoarthritis Drug (DMOAD) in Rats

A model for assessing therapeutic effect of p75NTR-Fc molecules in OA rats have been optimised. Briefly, OA has been induced injecting a low dose of MIA (0.3 mg) in either right/left knee of a rat. After 4 weeks, when signs of arthritis where visible, animals have been treated IV with p75NTR-Fc molecules, anti-NGF or vehicle as control. These have been dosed 5 times every 5 days. Half of animals per group have been then sacrificed and affected knees have been subjected to IHC (H&E, Saffron-O staining) and scored for OA pathology grade. The remaining half animals have been kept for an extra 28 days without treatment and then sacrificed and subjected to IHC as above. Synovial fluid from affected knees at terminal day have been collected for pK (active molecule and NGF) as well as biomarkers analyses. Sera at different timepoints have been collected to assess active compound and NGF systemic levels.

### Example 12—Human p75NTR ECD Stalk Modification—Protein Constructs and CHO-s Transfection/Expression

The amino acid sequences for protein constructs are listed below. Amino acid sequences are provided in the Sequences section
PetML 308—Human p75NTR ECD full stalk IgGI (SEQ ID NO:81)
PetML 309 —Human p75NTR ECD partial stalk IgGI (SEQ ID NO:82)
PetML 319 —Human p75NTR ECD no stalk (SEQ ID NO:83)

For protein production, DNA constructs were generated to encode chimeric Fc fusion protein comprising selected human IgG constant regions (between hinge and C-terminus) fused to the extracellular domain of human p75 either containing (PetML308—full stalk) or lacking predicted γ-secretase sites only (PetML309—partial stalk) or lacking O-glycosylation and γ-secretase sites (PetML319—no stalk).

Both the human IgG1 Fc domain and the p75 extracellular domain (res 31-194 from UniProtKB) were synthesised. Both genes were PCR amplified using Q5 high fidelity DNA polymerase (using specific primers including overlapping regions to allow assembly) and assembled into mammalian expression vector PetML319var using NEBuilder HIFI DNA Assembly (New England Biolabs). In the expression vector, the fusion protein chain and the antibiotic resistant gene expression units are flanked by DNA transposon piggyBac terminal inverted repeats to mediate stable integration into host cells in the presence of piggyBac transposase. The expression vector was then transfected into a suitable mammalian cell line such as CHO cells together with PiggyBac transposase to obtain stable expression. For fusion protein production, 1×10⁶/mL selected CHO cells are seeded in 800 mL culture media (F17+4 mM 1-Gln+0.3% P188+1:500 ACA) and incubated at 32° C., 8% CO₂ with shaking at 130 rpm. 2% HyClone Cell Boost 7a supplement+0.2% HyClone Cell Boost 7b supplement 2 mM glucose is added to the media daily from the 4^{th} day of overproduction. Culture supernatants are collected on day 10 and the protein concentration is determined using surface plasmon resonance using protein A chip (Biacore 8K, Cytiva Life Sciences).

Typically, human p75-Fc molecules showed peak of expression at 10 day in production reaching between 20 to 60 mg/L, with no stalk molecule showing best performances followed by partial stalk and full stalk.
PetML308: 20 mg/L
PetML309: 30 mg/L
PetML319: 60 mg/L

### Example 13—Human p75-Fc Molecules Purification

Cell suspensions from PetML319, PetML308 or PetML309 stable transfected clones, cultured as described for at least 7 days, were filtered using 0.22 um filters after being incubated for 10 minutes with Sartoclear Dynamics® Lab V (SDLV-0500-20C—E). Cleared supernatants have been loaded into Mabselect sure LX prepacked 20 mL column (17547402), pre-equilibrated with PBS. Column has been washed with 40 mLs of PBS (2CV) and then fusion proteins have been eluted using gradient (0-100% in 2CV) of 0.1M Glycine pH2.7. Fractionations containing fusion proteins have been pooled together and neutralised with 100 mM TRIS pH8 (final concentration).

Neutralised fusion protein pooled fractions have been concentrated till 5 mL and loaded into PBS pre-equilibrated HiLoad 16/600 Superdex 200 pg (28989335) as second step purification. Monomeric fractions (based on previously analysed protein standards' retention times) were pooled and protein concentration was assessed using NanoDrop™ One (Thermo Scientific™).

Around 10-30 mg/L of purified product was obtained following the above mentioned protocol. No difference in terms of purification recovery between wt and variants have been reported, differences are due to different titers.
PetML308: 10 mg/L after 2 steps purification
PetML309: 12 mg/L after 2 steps purification
PetML319: 30 mg/L after 2 steps purification

### Example 14—HPLC Analytical Chromatography

Purified material purity was assessed using both Size Exclusion Chromatography (SEC), for oligomerisation analyses, and cation exchange chromatography (SCX) for charge variants analyses.

HPLC-SEC chromatography (column: BioResolve SEC mAb 200A, 2.5 um column WATERS) was performed using ACQUITY H-class Bio from WATERS using PBS as mobile phase with isocratic 0.575 mL/min flow rate.

HPLC-SCX chromatography (column: BioResolve SCX mAb Column, 3 μm, 4.6 mm×100 mm) was performed using ACQUITY H-class Bio from WATERS using MES pH5 as mobile phase with salt gradient used to separate charge variants at 0.9 mL/min flow rate.

10 uL of each samples have been injected into both H-SEC/H-SCX using the above-mentioned protocol. Percentage of monomeric species and Area (indicative of protein concentration) were determined for each molecule.

PetML319 showed very high purity (more than 99%) by HSEC and few charge variants (potentially corresponding to different glycoforms) were observed by HSCX, while PetML309 and 308 showed presence of aggregates, leading to the idea that this stalk region can negatively affect developability of these molecules.

### Example 15—Protein A Binding Affinity Validation

Purified fusion proteins in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to Protein A was assessed using Biacore 8K (Cytiva).

Briefly, Sensor Chip Protein A (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

Fusion protein dilutions were prepared diluting PetML319, PetML308 or PetML309from 1 uM to 4 nM (6 concentrations with 1:3 dilutions) in Running Buffer and kinetics was assessed using single cycle kinetics method (Biacore Assay Handbook, Cytiva). Kinetics and/or Affinity quantification have been performed using Biacore Insight following standard analyses methods.

The results show that all molecules have proficient protein A binding.

### Example 16—Human and Rat Nerve Growth Factor (h-rNGF) Binding Affinity Determination

Purified fusion proteins in PBS were concentrated using centrifugal concentrators (Sartorious—VS02H22) to 5 mg/mL Protein concentration was assessed using UV absorbance at 280 nm with NanoDrop™ One (Thermo Scientific™).

Binding affinity of fusion proteins to human and rat NGF was assessed using Biacore 8K (Cytiva).

Briefly, Protein A Sensor Chip (Cytiva) was docked into Biacore 8K, equilibrated for 30′ at RT and then Running Buffer (10 mM HEPES pH7.4 150 mM NaCl 3 mM EDTA and 0.005% Tween20) was applied to the SPR chip surface.

PetML319, PetML308 or PetML309 was diluted into running buffer at 6 nM concentration. These have been immobilised using 90 sec association at 10 uL/min as capturing step, followed by injection of running buffer to remove any unbound product.

Human and rat NGF (from Bio-Techne Ltd—556-NG/CF/256-GF-100/CF) was diluted in Running Buffer at 100 nM with 1:2 further dilutions down to 4.68 nM. Kinetics were assessed using multi-cycle kinetics with capture step method (30 sec association—300 sec dissociation) followed by regeneration step (0.1M Glycine pH2.2 contact time 60 sec FR 30 uL/min). Kinetics quantification have been performed using Biacore Insight following standard analyses methods (Table 5).

TABLE 5

| ECD stalk | | | | | Rmax |
|---|---|---|---|---|---|
| variant | ligand | ka (1/Ms) | kd (1/s) | KD (M) | (RU) |
| PetML308 | hNGF | 3.42E+02 | 1.94E−03 | 5.68E−06 | 15.9 |
| PetML309 | hNGF | 1.11E+06 | 2.75E−02 | 2.48E−08 | 18.4 |
| PetML319 | hNGF | 8.86E+06 | 6.59E−04 | 7.43E−11 | 76.1 |

Results showed subnanomolar KD for both human and rat NGF with PetML319, while slightly lower affinity for PetML309 and 308 respectively (Table 6).

TABLE 6

| Antibody/ | KD | |
|---|---|---|
| Fc-protein | (nM) | RMAX |
| Anti-NGF mAb | 0.0073 | 837.1 |
| PetML319 | 0.358 | 370.8 |
| PetML308 | 8.78 | 345.4 |
| PetML309 | 2.31 | 131.9 |
| PetML309 | 2.31 | 131.9 |

### Example 17—Unfolding and Oligomerisation Determination

Purified fusion proteins in PBS were concentrated using centrifugal concentrators (Sartorious—VS02H22) to 3 mg/mL Protein concentration was assessed using UV absorbance at 280 nm with NanoDrop™ One (Thermo Scientific™).

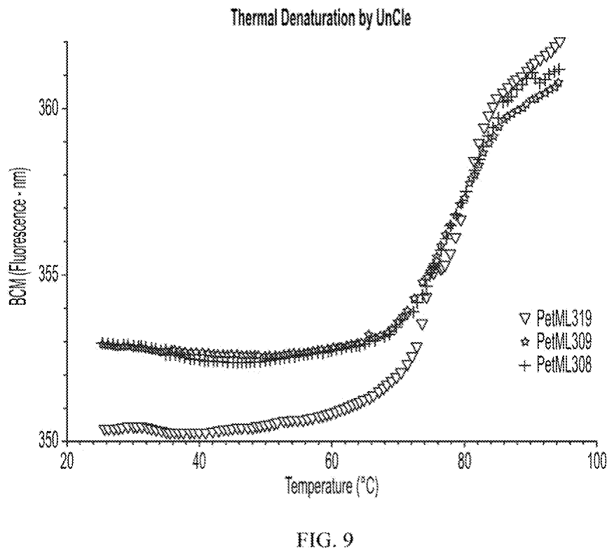

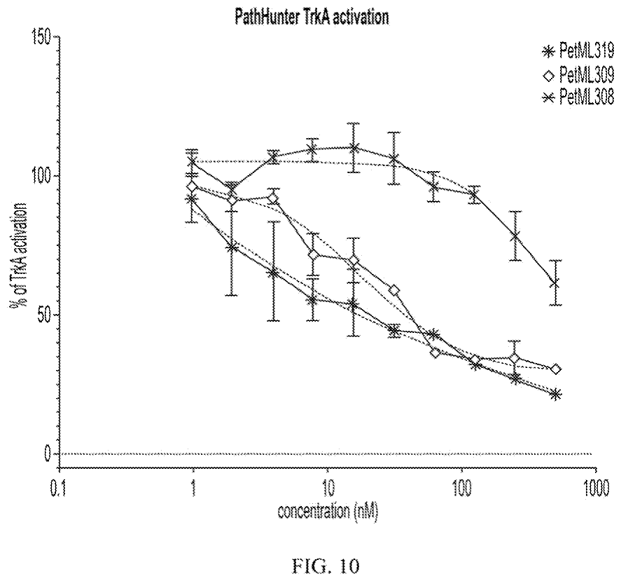

Tm and Tagg analyses have been performed on UnCle from Unchained labs using standard protocol. (FIG. 9)

Briefly, 10 uL of fusion protein have been used to determine unfolding and aggregation events during a temperature ramp (from 25° C. till 95° C.). All molecules showed no aggregation up to 95° C. with Tm1 around 76° C. (Table 7)

TABLE 7

| ECD stalk variant | Tm (° C.) |
|---|---|
| PetML308 | 75.6 |
| PetML309 | 73.6 |
| PetML319 | 81.04 |

### Example 18—PathHunter® eXpress Receptor Tyrosine Kinase Functional Assay Kit

To further prove that our molecules are able to inhibit NGF pathway activation in-vitro, we've taken advantage of this kit where a cell line has been engineered to have TrkA intracellular domain fused with a small complementing fragment of β-Gal. The larger portion of β-gal, termed EA for ‘enzyme acceptor’, is fused to proteins containing phospho-tyrosine binding domains. Ligand-induced activation of the receptor causes either homo or hetero-dimerization of the receptor which results in cross-phosphorylation. The SH2-EA fusion protein then specifically binds the phosphorylated receptor resulting in complementation of the two fragments of β-gal and formation of a functional enzyme. β-gal activity is then quantitatively detected using the chemiluminescent substrate in the PathHunter Detection Kit.

Briefly, 3 uM concentration till 91.25 nM of PetML119wt, var and positive control anti-NGF mAB (Bedinvetmab) have been used while NGF was used at 2 nM. Luminescence readings showed that, similarly to what have been seen in TF-1 assay, anti-NGF mAb has a distinct sigmoidal shape inhibition curve reaching 100% TrkA inhibition. In contrast to anti-NGF but consistently again with TF-1 assay, PetML119 molecules showed shallower inhibition curve that don't reach 100% inhibition. Also, high affinity NGF PetML119 molecules showed increased max inhibition (still not 100%) and reduced IC50, while low affinity NGF PetML119 molecules showed very little inhibition, again consistently with TF-1 inhibition assay.

Altogether these results suggest that anti-NGF mAb Bedinvetmab differs from human p75-Fc molecules both on inhibition concentration and amplitude of inhibition. Moreover, no stalk molecule showed better performances than partial and full stalk, probably due to reduced stability and binding capacity as described before.

TABLE 8

| Best-fit values | Bedinvetmab | PetML308 | PetML309 | PetML319 |
|---|---|---|---|---|
| Bottom (%) | 2.111 | 44.48 | 28.73 | 9.5 |
| Top (%) | 93.48 | 105.2 | 97.92 | Unstable |
| IC50 (nM) | 1.138 | 282.1 | 18.70 | 8.04 |

Sequences

Canine p75NTR protein (SEQ ID NO: 1)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVDPCLPCTVCEDTERQLRECTRWADAECEEIPGRWITRSTPSE
DSDSTAPSTEEPELPPDQEIIASTMADVVTTVMGSSQPVVTRGTADNLIPVYCSILAAVV
VGLVAYIAFKRWNSCKQNKQGANSRPVNQTPPPEGEKLHSDSGISVDSQSLHDQQPHTQ
TAAGQALKGDGGLYSSLPPAKREEVEKLLNGSAGDTWRHLAGELGYQPEHIDSFTHEA
CPARALLASWAAQDSATLDALLAALRRIQRADIVESLCSESTATSPV
ECD is underlined.
Canine p75NTR nucleic acid sequence (SEQ ID NO: 2)
ATGGACGGGCCGCGCCTGCTGCTGCTGCTGCTGCTGCTCCTGGGGGTGTCCCTTGGA
GGTGCCAAGGAGGCATGTCCCACTGGCCTGTACACCCACAGCGGCGAGTGCTGCAA
AGCCTGCAATCTGGGTGAGGGGGTGGCCCAGCCTTGCGGAGCCAACCAGACCGTGT
GTGAGCCCTGCCTGGACAGCGTGACCTTCTCGGACGTGGTGAGCGCCACCGAGCCG
TGCAAGCCGTGCACCGAGTGCGTGGGGCTGCAGAGCATGTCGGCGCCGTGCGTGGA
GGCGGACGACGCCGTGTGCCGCTGCGCCTACGGCTACTACCAGGACGAGACGACGG
GCCGCTGCGAGGCGTGCCGCGTGTGCGAGGCGGGCTCGGGGCTCGTGTTCTCGTGCC
AGGACAGGCAGAACACCGTGTGCGAGGAGTGTCCCGACGGCACGTACTCCGACGAG
GCCAACCACGTGGACCCGTGCCTGCCCTGCACCGTGTGCGAGGACACCGAGCGCCA
GCTGCGCGAGTGCACGCGCTGGGCCGACGCCGAGTGCGAGGAGATCCCTGGCCGTT
GGATTACCCGGTCCACACCCTCAGAGGACTCGGACAGCACCGCCCCCAGCACAGAG
GAGCCAGAGCTACCTCCAGATCAAGAAATCATAGCCAGCACCATGGCAGATGTGGT
GACCACAGTGATGGGCAGCTCTCAGCCTGTAGTGACCCGAGGAACCGCTGACAACC
TCATCCCTGTCTACTGCTCCATCCTGGCCGCCGTGGTTGTGGGCTTAGTGGCCTACAT
TGCCTTCAAGAGGTGGAACAGCTGCAAGCAGAACAAGCAAGGAGCCAACAGCCGG
CCCGTGAACCAGACGCCTCCGCCGGAGGGAGAAAAGCTCCACAGTGACAGTGGCAT
CTCTGTGGACAGCCAGAGCCTGCATGACCAGCAGCCCCACACACAGACGGCCGCAG
GCCAGGCCCTCAAGGGGGATGGAGGTCTCTACAGCAGCCTGCCACCAGCCAAGCGG
GAGGAGGTGGAGAAGCTGCTCAATGGCTCTGCGGGGGACACCTGGCGGCACCTGGC
AGGTGAGCTGGGCTACCAGCCTGAGCACATAGACTCCTTCACCCACGAGGCCTGCC
CAGCCCGAGCCCTGCTTGCCAGCTGGGCCGCCCAGGACAGCGCGACGCTCGACGCC
CTCCTGGCTGCTCTGCGCCGCATCCAGCGAGCCGACATCGTGGAGAGCCTGTGTAGC
GAGTCCACGGCCACGTCTCCAGTGTGA
Leader sequence is underlined.
Feline p75NTR protein (SEQ ID NO: 3)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVESCQDRQNT
VCEECPDGTYSDEANHVDPCLPCTVCEDTERQLRECTRWADAECEEIPGRWITRSTPSE
GSDSTAPSTEEPEVPPEQDLIASTVADVVTTVMGSSQPVVTRGTADNLIPVYCSILAAVV
VGLVAYIAFKRWNSCKQDKQGANSRPVNQTPPPEGEKLHSDSGISVDSQSLHDQQSHTQ
TAAGQALKGDGGLYSSLPSAKREEVEKLLNGSAGDTWRHLAGELGYQPEHIDSFTREA
CPARALLASWAAQDSATLDALLAALRRIQRADIVESLCSESTATSPV
ECD is underlined.
Feline p75NTR nucleic acid sequence (SEQ ID NO: 4)
ATGGACGGGCCGCGCCCGCTGCTGCTGCTGTTGCCGCTGCTCCTGGGGGTGTCCCTT
GGAGGTGCCAAGGAGGCATGTCCCACGGGCCTGTTCACCCACAGCGGCGAGTGCTG
TAAAGCCTGCAACCTGGGAGAGGGCGTAGCCCAGCCTTGCGGAGCCAACCAGACCG
TGTGTGAGCCCTGCCTGGACAGCGTGACCTTCTCGGACGTGGTGAGCGCCACGGAG
CCGTGCAAGCCGTGCACCGAGTGCGTGGGCCTGCAGAGCATGTCGGCGCCGTGCGT
GGAGGCCGACGACGCCGTGTGTCGCTGCGCCTACGGCTACTACCAGGACGAGACGA
CGGGCCGCTGCGAGGCGTGCCGCGTGTGCGAGGCGGGCTCCGGCCTGGTGTTCTCGT
GCCAGGACCGGCAGAATACCGTGTGCGAGGAGTGTCCCGACGGCACGTACTCGGAC
GAGGCCAACCACGTGGACCCGTGCCTGCCCTGCACCGTGTGCGAGGACACCGAGCG
CCAGCTGCGCGAGTGCACGCGCTGGGCCGACGCCGAGTGCGAGGAGATCCCTGGCC
GTTGGATTACTCGGTCTACACCTTCGGAGGGCTCGGACAGCACCGCCCCCAGCACGG
AGGAGCCAGAGGTACCTCCAGAGCAAGACCTCATAGCCAGCACGGTGGCAGATGTG
GTGACCACAGTGATGGGCAGCTCTCAGCCCGTAGTGACCCGAGGCACCGCCGACAA
CCTCATCCCTGTCTATTGTTCCATCCTGGCCGCTGTGGTTGTGGGCCTGGTGGCCTAC
ATTGCCTTCAAGAGGTGGAACAGCTGCAAACAGGACAAGCAAGGCGCCAACAGCCG
GCCCGTGAACCAGACGCCCCCGCCCGAGGGAGAAAAGCTCCACAGTGACAGTGGCA
TCTCTGTGGACAGCCAGAGCCTGCATGACCAGCAGTCCCACACGCAGACGGCCGCC
GGCCAGGCCCTCAAGGGGGACGGAGGTCTCTACAGCAGCCTGCCGTCAGCCAAGCG
GGAGGAGGTAGAGAAACTGCTCAACGGCTCTGCGGGGGACACGTGGCGGCACCTGG
CGGGCGAGCTGGGCTACCAGCCTGAGCACATAGACTCCTTCACCCGCGAGGCCTGC
CCAGCCCGGGCCCTGCTCGCCAGCTGGGCCGCCCAGGACAGCGCGACGCTCGACGC
CCTCCTGGCCGCCCTGCGCCGCATCCAGCGGGCCGACATCGTCGAGAGCCTGTGCAG
CGAGTCCACGGCCACGTCCCCGGTGTGA
Leader sequence is underlined.
Equine p75NTR protein (SEQ ID NO: 5)
KEVCPTDLYTHSGECCKACNLGEGVAQPCGANQTVCEPCLDSVTFSDVVSATEPCKPCT
ECVGLQSMSAPCVEADDAVCRCAYGYYQDETTGRCEACQVCEAGSGLVESCQDKQNT
VCEECPDGTYSDEANHVDPCLPCTVCEDTERQLRECTRWADAECEEIPSRWITRATPPE
GSDSTAPSTQEPEGPPEKDLVASTVADVVTTVMGSSQPVVTRGTTDNLIPVYCSILAAVV
VGLVAYIAFKRWNSCKQNKQGANSRPVNQTPPPEGEKLHSDSGISVDSQSLHDQQPHTQ
TAAGQALKGDGGLYSSLPLAKREEVEKLLNGSAGDTWRHLAGLVGQGLLRLELVSVFQ
GPAHGGMLPPATPSLQAPVWLGPEGCSEKWDQRGNAARRAGLRVWPMEGLSQV
ECD is underlined.
Equine p75NTR nucleic acid sequence (SEQ ID NO: 6)
ATGAGGGCAGGTGCCGCCGACTGCGCCATGGACGGACCGCGCCTTCTGCTGCTGCTT
CTGCTCTTGGGGGTGTGCCTGCTGGGAGGTGCCAAGGAGGTGTGCCCCACAGACCTG
TACACCCACAGCGGCGAGTGCTGCAAAGCCTGCAACCTGGGCGAGGGTGTGGCCCA
GCCTTGCGGAGCCAACCAGACTGTGTGTGAACCCTGCCTGGACAGCGTGACGTTCTC
GGACGTGGTGAGCGCCACAGAGCCATGTAAGCCGTGCACCGAGTGCGTGGGCCTGC
AGAGCATGTCGGCGCCATGCGTGGAGGCCGACGACGCGGTGTGCCGCTGCGCCTAT
GGCTACTACCAGGACGAGACGACGGGCCGCTGCGAGGCGTGCCAGGTGTGCGAGGC
GGGCTCGGGCCTCGTGTTCTCGTGCCAGGACAAGCAGAACACCGTGTGCGAGGAAT
GCCCCGACGGCACGTACTCCGACGAGGCCAACCACGTGGACCCGTGCCTGCCCTGC
ACCGTGTGCGAGGACACCGAGCGCCAGCTGCGAGAGTGCACGCGCTGGGCCGACGC
CGAGTGCGAGGAGATCCCCAGCCGTTGGATTACACGGGCCACGCCGCCGGAGGGCT
CAGACAGCACTGCCCCCAGCACCCAGGAGCCCGAGGGACCTCCAGAGAAAGACCTT
GTAGCCAGCACGGTGGCGGATGTGGTGACCACAGTGATGGGCAGCTCTCAGCCCGT
GGTGACCCGAGGCACCACGGACAACCTCATCCCCGTCTATTGCTCCATCCTGGCCGC
TGTGGTTGTGGGCCTTGTGGCCTACATCGCCTTCAAGAGGTGGAACAGCTGCAAGCA
GAACAAGCAAGGAGCCAACAGCCGACCCGTGAACCAGACACCACCACCCGAGGGA
GAAAAACTCCACAGCGACAGCGGCATCTCTGTGGACAGCCAGAGCCTGCATGACCA
GCAGCCTCACACACAGACAGCCGCAGGCCAGGCCCTCAAGGGAGATGGAGGCCTCT
ACAGCAGCCTGCCACTGGCCAAGAGGGAAGAGGTGGAGAAGCTACTCAATGGCTCC
GCAGGGGACACCTGGCGGCACCTGGCGGGTGAGCTGGGCTACCAGCCCGAGCACAT
AGACTCCTTCACCCACGAGGCCTGCCCCGTCCGCGCCCTGCTTGCCAGCTGGGCCGC
CCAGGACAGTGCGACATTCGATGCCCTCCTGACCGCCCTGCGCCGCATCCAGCGAGC
CGACATTGTCGAGAGCCTGTGCAGCGAGTCCACCGCCACATCCCCGGTGTGA
Leader sequence is underlined.
Canine p75NTR protein ECD (SEQ ID NO: 7)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVDPCLPCTVCEDTERQLRECTRWADAECEEIPGRWITRSTPSE
DSDSTAPSTEEPELPPDQEIIASTMADVVTTVMGSSQPVVTRGTADN
The wt ECD region includes the stalk region (underlined) and alpha and
gamma secretase cleavage 3′ of the stalk region (in bold)
Canine p75NTR ECD nucleic acid sequence (SEQ ID NO: 8)
AAGGAGGCATGTCCCACTGGCCTGTACACCCACAGCGGCGAGTGCTGCAAAGCCTG
CAATCTGGGTGAGGGGGTGGCCCAGCCTTGCGGAGCCAACCAGACCGTGTGTGAGC
CCTGCCTGGACAGCGTGACCTTCTCGGACGTGGTGAGCGCCACCGAGCCGTGCAAG
CCGTGCACCGAGTGCGTGGGGCTGCAGAGCATGTCGGCGCCGTGCGTGGAGGCGGA
CGACGCCGTGTGCCGCTGCGCCTACGGCTACTACCAGGACGAGACGACGGGCCGCT
GCGAGGCGTGCCGCGTGTGCGAGGCGGGCTCGGGGCTCGTGTTCTCGTGCCAGGAC
AGGCAGAACACCGTGTGCGAGGAGTGTCCCGACGGCACGTACTCCGACGAGGCCAA
CCACGTGGACCCGTGCCTGCCCTGCACCGTGTGCGAGGACACCGAGCGCCAGCTGC
GCGAGTGCACGCGCTGGGCCGACGCCGAGTGCGAGGAGATCCCTGGCCGTTGGATT
ACCCGGTCCACACCCTCAGAGGACTCGGACAGCACCGCCCCCAGCACAGAGGAGCC
AGAGCTACCTCCAGATCAAGAAATCATAGCCAGCACCATGGCAGATGTGGTGACCA
CAGTGATGGGCAGCTCTCAGCCTGTAGTGACCCGAGGAACCGCTGACAAC
Canine ECD of p75NTR stalk region protein (SEQ ID NO: 9)
WITRSTPSEDSDSTAPSTEEPELPPDQEIIASTMADVVTTVM
Canine ECD of p75NTR stalk region nucleic acid sequence (SEQ ID NO: 10)
TGGATTACCCGGTCCACACCCTCAGAGGACTCGGACAGCACCGCCCCCAGCACAGA
GGAGCCAGAGCTACCTCCAGATCAAGAAATCATAGCCAGCACCATGGCAGATGTGG
TGACCACAGTGATG
Canine p75NTR ECD-canine IgGB wt Fc protein fusion (SEQ ID NO: 11)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFSCQDRQNTVCEECPDGTYSDEANHVDPCLPCTVCEDTERQLRECTRW
ADAECEEIPGGGGRENGRVPRPPDCPKCPAPEMLGGPSVFIFPPKPKDTLLIARTPEVTC
VVVDLDPEDPEVQISWFVDGKQMQTAKTQPREEQFNGTYRVVSVLPIGHQDWLKGKQF
TCKVNNKALPSPIERTISKARGQAHQPSVYVLPPSREELSKNTVSLTCLIKDFFPPDIDVE
WQSNGQQEPESKYRTTPPQLDEDGSYFLYSKLSVDKSRWQRGDTFICAVMHEALHNHY
TQKSLSHSPGK
Signal peptide-Canine p75-ECD-Linker GGGG-Canine Fc-B-wt
Canine p75NTR ECD-canine IgGB wt Fc nucleic acid sequence (SEQ ID NO: 12)
ATGGAATGGTCCTGGGTGTTCCTGTTCTTCCTGTCCGTGACCACCGGCGTGCACTCCA
AAGAGGCTTGTCCTACCGGCCTGTACACCCACTCTGGCGAGTGTTGCAAGGCCTGTA
ATCTCGGCGAAGGCGTGGCACAACCTTGTGGCGCTAATCAGACAGTGTGCGAGCCTT
GCCTGGACTCCGTGACCTTCTCTGATGTGGTGTCTGCCACCGAGCCATGCAAGCCTT
GTACCGAGTGTGTGGGCCTGCAGTCCATGTCTGCCCCTTGTGTGGAAGCCGACGACG
CCGTGTGTAGATGTGCCTACGGCTACTACCAGGACGAGACAACCGGAAGATGCGAG
GCCTGCAGAGTGTGTGAAGCTGGCTCTGGACTGGTGTTCTCCTGCCAAGACAGACAG
AACACCGTGTGCGAGGAATGCCCTGACGGCACCTACTCTGATGAGGCCAATCACGT
GGACCCCTGCCTGCCTTGTACTGTGTGCGAAGATACCGAGCGGCAGCTGCGCGAGT
GTACCAGATGGGCTGATGCCGAGTGCGAAGAGATCCCTGGAGGTGGCGGACGCGAG
AATGGCAGAGTGCCTAGACCTCCTGACTGCCCTAAGTGCCCTGCTCCTGAAATGCTC
GGCGGACCCTCCGTGTTCATCTTCCCACCTAAGCCTAAGGACACCCTGCTGATCGCT
CGGACCCCTGAAGTGACATGCGTGGTGGTGGATCTGGACCCCGAGGATCCTGAGGT
GCAGATCAGTTGGTTCGTGGACGGCAAGCAGATGCAGACCGCTAAGACCCAGCCTA
GAGAGGAACAGTTCAACGGCACCTACAGAGTGGTGTCTGTGCTGCCTATCGGCCAC
CAGGATTGGCTGAAGGGCAAGCAGTTTACCTGCAAAGTGAACAACAAGGCCCTGCC
TTCTCCAATCGAGCGGACCATCTCTAAGGCCAGAGGCCAGGCTCATCAGCCTTCCGT
GTATGTCCTGCCACCTAGCCGCGAGGAACTGTCCAAGAACACCGTGTCTCTGACCTG
CCTGATCAAGGACTTCTTCCCTCCTGACATCGACGTGGAATGGCAGTCCAACGGCCA
GCAAGAGCCCGAGTCTAAGTACCGGACAACCCCTCCACAGCTGGACGAGGACGGCT
CCTACTTCCTGTACTCCAAGCTGTCCGTGGACAAGTCTCGGTGGCAGAGAGGCGACA
CCTTCATCTGTGCTGTGATGCACGAGGCCCTGCACAACCACTACACCCAGAAGTCCC
TGTCTCACTCCCCTGGCAAGTGA
Leader sequence is underlined.
Canine p75NTR ECD-canine Fc YTE protein fusion (SEQ ID NO: 13)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFSCQDRQNTVCEECPDGTYSDEANHVDPCLPCTVCEDTERQLRECTRW
ADAECEEIPGGGGRENGRVPRPPDCPKCPAPEMLGGPSVFIFPPKPKDTLYITREPEVTC
VVVDLDPEDPEVQISWFVDGKQMQTAKTQPREEQFNGTYRVVSVLPIGHQDWLKGKQF
TCKVNNKALPSPIERTISKARGQAHQPSVYVLPPSREELSKNTVSLTCLIKDFFPPDIDVE
WQSNGQQEPESKYRTTPPQLDEDGSYFLYSKLSVDKSRWQRGDTFICAVMHEALHNHY
TQKSLSHSPGK
Signal peptide-Canine p75-ECD-Linker GGGG-Canine Fc-B-YTE
Canine p75NTR ECD-Fc YTE nucleic acid sequence (SEQ ID NO: 14)
ATGGAATGGTCCTGGGTGTTCCTGTTCTTCCTGTCCGTGACCACCGGCGTGCACTCCA
AAGAGGCTTGTCCTACCGGCCTGTACACCCACTCTGGCGAGTGTTGCAAGGCCTGTA
ATCTCGGCGAAGGCGTGGCACAACCTTGTGGCGCTAATCAGACAGTGTGCGAGCCTT
GCCTGGACTCCGTGACCTTCTCTGATGTGGTGTCTGCCACCGAGCCATGCAAGCCTT
GTACCGAGTGTGTGGGCCTGCAGTCCATGTCTGCCCCTTGTGTGGAAGCCGACGACG
CCGTGTGTAGATGTGCCTACGGCTACTACCAGGACGAGACAACCGGAAGATGCGAG
GCCTGCAGAGTGTGTGAAGCTGGCTCTGGACTGGTGTTCTCCTGCCAAGACAGACAG
AACACCGTGTGCGAGGAATGCCCTGACGGCACCTACTCTGATGAGGCCAATCACGT
GGACCCCTGCCTGCCTTGTACTGTGTGCGAAGATACCGAGCGGCAGCTGCGCGAGT
GTACCAGATGGGCTGATGCCGAGTGCGAAGAGATCCCTGGAGGTGGCGGACGCGAG
AATGGCAGAGTGCCTAGACCTCCTGACTGCCCTAAGTGCCCTGCTCCTGAAATGCTC
GGCGGACCCTCCGTGTTCATCTTCCCACCTAAGCCTAAGGACACCCTGTATATCACT
CGGGAACCTGAAGTGACATGCGTGGTGGTGGATCTGGACCCCGAGGATCCTGAGGT
GCAGATCAGTTGGTTCGTGGACGGCAAGCAGATGCAGACCGCTAAGACCCAGCCTA
GAGAGGAACAGTTCAACGGCACCTACAGAGTGGTGTCTGTGCTGCCTATCGGCCAC
CAGGATTGGCTGAAGGGCAAGCAGTTTACCTGCAAAGTGAACAACAAGGCCCTGCC
TTCTCCAATCGAGCGGACCATCTCTAAGGCCAGAGGCCAGGCTCATCAGCCTTCCGT
GTATGTCCTGCCACCTAGCCGCGAGGAACTGTCCAAGAACACCGTGTCTCTGACCTG
CCTGATCAAGGACTTCTTCCCTCCTGACATCGACGTGGAATGGCAGTCCAACGGCCA
GCAAGAGCCCGAGTCTAAGTACCGGACAACCCCTCCACAGCTGGACGAGGACGGCT
CCTACTTCCTGTACTCCAAGCTGTCCGTGGACAAGTCTCGGTGGCAGAGAGGCGACA
CCTTCATCTGTGCTGTGATGCACGAGGCCCTGCACAACCACTACACCCAGAAGTCCC
TGTCTCACTCCCCTGGCAAGTGA
Leader sequence is underlined.
IgG-A (SEQ ID NO: 15)
MEFVLGWVFLVAILQGVQGEVQLVESGGDLVKPAGSLRLSCVASGFTFSNNAMNWVR
QAPGKGLQWVAGINSGGSTASADAVKGRFTISRDNAKNTVYLQMNSLTAEDTAVYYC
AKVIGNWIATSDLDYWGQGTLVIVSSASTTAPSVFPLAPSCGSTSGSTVALACLVSGYFP
EPVTVSWNSGSLTSGVHTFPSVLQSSGLYSLSSMVTVPSSRWPSETFTCNVVHPASNTKV
DKPVFNECRCTDTPPCPVPEPLGGPSVLIFPPKPKDILRITRTPEVTCVVLDLGREDPEVQI
SWFVDGKEVHTAKTQSREQQFNGTYRVVSVLPIEHQDWLTGKEFKCRVNHIDLPSPIER
TISKARGRAHKPSVYVLPPSPKELSSSDTVSITCLIKDFYPPDIDVEWQSNGQQEPERKHR
MTPPQLDEDGSYFLYSKLSVDKSRWQQGDPFTCAVMHETLQNHYTDLSLSHSPGK
IgG-B (SEQ ID NO: 16)
MEFVLGWVFLVAILQGVQGEVQLVESGGDLVKPAGSLRLSCVASGFTFSNNAMNWVR
QAPGKGLQWVAGINSGGSTASADAVKGRFTISRDNAKNTVYLQMNSLTAEDTAVYYC
AKVIGNWIATSDLDYWGQGTLVIVSSASTTAPSVFPLAPSCGSTSGSTVALACLVSGYFP
EPVTVSWNSGSLTSGVHTFPSVLQSSGLYSLSSMVTVPSSRWPSETFTCNVAHPASKTKV
DKPVPKRENGRVPRPPDCPKCPAPEMLGGPSVFIFPPKPKDTLLIARTPEVTCVVVDLDP
EDPEVQISWFVDGKQMQTAKTQPREEQFNGTYRVVSVLPIGHQDWLKGKQFTCKVNN
KALPSPIERTISKARGQAHQPSVYVLPPSREELSKNTVSLTCLIKDFFPPDIDVEWQSNGQ
QEPESKYRTTPPQLDEDGSYFLYSKLSVDKSRWQRGDTFICAVMHEALHNHYTQKSLSH
SPGK
IgG-C (SEQ ID NO: 17)
MEFVLGWVFLVAILQGVQGEVQLVESGGDLVKPAGSLRLSCVASGFTFSNNAMNWVR
QAPGKGLQWVAGINSGGSTASADAVKGRFTISRDNAKNTVYLQMNSLTAEDTAVYYC
AKVIGNWIATSDLDYWGQGTLVIVSSASTTAPSVFPLAPSCGSQSGSTVALACLVSGYIP
EPVTVSWNSGSLTSGVHTFPSILQSSGLYSLSSMVTVPSSRWPSETFTCNVAHPATNTKV
DKPVVKECECKCNCNNCPCPGCGLLGGPSVFIFPPKPKDILVTARTPTVTCVVVDLDPEN
PEVQISWFVDSKQVQTANTQPREEQSNGTYRVVSVLPIGHQDWLSGKQFKCKVNNKAL
PSPIEEIISKTPGQAHQPNVYVLPPSRDEMSKNTVTLTCLVKDFFPPEIDVEWQSNGQQEP
ESKYRMTPPQLDEDGSYFLYSKLSVDKSRWQRGDTFICAVMHEALHNHYTQKSLSHSP
GK
IgG-D (SEQ ID NO: 18)
MEFVLGWVFLVAILQGVQGEVQLVESGGDLVKPAGSLRLSCVASGFTFSNNAMNWVR
QAPGKGLQWVAGINSGGSTASADAVKGRFTISRDNAKNTVYLQMNSLTAEDTAVYYC
AKVIGNWIATSDLDYWGQGTLVIVSSASSTAPSVFPLAPSCGSTSGSTVALACLVSGYFP
EPVTVSWNSGSLTSGVHTFPSVLKSSGLYSLSSMVTVPSSRLPSETFTCNVVHPATNTKV
DKPVPKESTCKCISPCPVPESLGGPSVFIFPPKPKDILRITRTPEVTCVVLDLGREDPEVQIS
WFVDGKEVHTAKTQPREQQFNSTYRVVSVLPIEHQDWLTGKEFKCRVNHIGLPSPIERTI
SKARGQAHQPGVYVLPPSPKELSSSDTVTLTCLIKDFFPPEIDVEWQSNGQPEPESKYHT
TAPQLDEDGSYFLYSKLSVDKSRWQQGDPFTCAVMHEALQNHYTDLSLSHSPGK
DOGA constant region (SEQ ID NO: 19)
FNECRCTDTPPCPVPEPLGGPSVLIFPPKPKDILRITRTPEVTCVVLDLGREDPEVQISWFV
DGKEVHTAKTQSREQQFNGTYRVVSVLPIEHQDWLTGKEFKCRVNHIDLPSPIERTISKA
RGRAHKPSVYVLPPSPKELSSSDTVSITCLIKDFYPPDIDVEWQSNGQQEPERKHRMTPP
QLDEDGSYFLYSKLSVDKSRWQQGDPFTCAVMHETLQNHYTDLSLSHSPGK
DOGB constant region (SEQ ID NO: 20)
RENGRVPRPPDCPKCPAPEMLGGPSVFIFPPKPKDTLLIARTPEVTCVVVDLDPEDPEVQI
SWFVDGKQMQTAKTQPREEQFNGTYRVVSVLPIGHQDWLKGKQFTCKVNNKALPSPIE
RTISKARGQAHQPSVYVLPPSREELSKNTVSLTCLIKDFFPPDIDVEWQSNGQQEPESKY
RTTPPQLDEDGSYFLYSKLSVDKSRWQRGDTFICAVMHEALHNHYTQKSLSHSPGK
DOGB-YTE constant region (SEQ ID NO: 21)
RENGRVPRPPDCPKCPAPEMLGGPSVFIFPPKPKDTLYITREPEVTCVVVDLDPEDPEVQI
SWFVDGKQMQTAKTQPREEQFNGTYRVVSVLPIGHQDWLKGKQFTCKVNNKALPSPIE
RTISKARGQAHQPSVYVLPPSREELSKNTVSLTCLIKDFFPPDIDVEWQSNGQQEPESKY
RTTPPQLDEDGSYFLYSKLSVDKSRWQRGDTFICAVMHEALHNHYTQKSLSHSPGK
DOGC constant region (SEQ ID NO: 22)
ECECKCNCNNCPCPGCGLLGGPSVFIFPPKPKDILVTARTPTVTCVVVDLDPENPEVQIS
WFVDSKQVQTANTQPREEQSNGTYRVVSVLPIGHQDWLSGKQFKCKVNNKALPSPIEEI
ISKTPGQAHQPNVYVLPPSRDEMSKNTVTLTCLVKDFFPPEIDVEWQSNGQQEPESKYR
MTPPQLDEDGSYFLYSKLSVDKSRWQRGDTFICAVMHEALHNHYTQKSLSHSPGK
DOGD constant region (SEQ ID NO: 23)
ESTCKCISPCPVPESLGGPSVFIFPPKPKDILRITRTPEVTCVVLDLGREDPEVQISWFVDG
KEVHTAKTQPREQQFNSTYRVVSVLPIEHQDWLTGKEFKCRVNHIGLPSPIERTISKARG
QAHQPGVYVLPPSPKELSSSDTVTLTCLIKDFFPPEIDVEWQSNGQPEPESKYHTTAPQLD
EDGSYFLYSKLSVDKSRWQQGDPFTCAVMHEALQNHYTDLSLSHSPGK
CAT_IGG1V1 constant region (SEQ ID NO: 24)
TDHPPGPKPCDCPKCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNSKSLPSPIERTI
SKAKGQPHEPQVYVLPPAQEELSRNKVSVTCLIKSFHPPDIAVEWEITGQPEPENNYRTT
PPQLDSDGTYFVYSKLSVDRSHWQRGNTYTCSVSHEALHSHHTQKSLTQSPGK
CAT_IGG1V2 constant region (SEQ ID NO: 25)
TDHPPGPKPCDCPKCPPPEMLGGPSIFIFPPKPKDTLSISRTPEVTCLVVDLGPDDSDVQIT
WFVDNTQVYTAKTSPREEQFNSTYRVVSVLPILHQDWLKGKEFKCKVNSKSLPSPIERTI
SKAKGQPHEPQVYVLPPAQEELSRNKVSVTCLIKSFHPPDIAVEWEITGQPEPENNYRTT
PPQLDSDGTYFVYSKLSVDRSHWQRGNTYTCSVSHEALHSHHTQKSLTQSPGK
CAT_IGG2 constant region (SEQ ID NO: 26)
KTASTIESKTGEGPKCPVPEIPGAPSVFIFPPKPKDTLSISRTPEVTCLVVDLGPDDSNVQIT
WFVDNTEMHTAKTRPREEQFNSTYRVVSVLPILHQDWLKGKEFKCKVNSKSLPSAMER
TISKAKGQPHEPQVYVLPPTQEELSENKVSVTCLIKGFHPPDIAVEWEITGQPEPENNYQT
TPPQLDSDGTYFLYSRLSVDRSHWQRGNTYTCSVSHEALHSHHTQKSLTQSPGK
HORSE_IGHG1 constant region (SEQ ID NO: 27)
VIKECNGGCPAECLQVGPSVFIFPPKPKDVLMISRTPTVTCVVVDVGHDFPDVQFNWYV
DGVETHTATTEPKQEQFNSTYRVVSVLPIQHKDWLSGKEFKCKVNNKALPAPVERTISK
PTGQPREPQVYVLAPHRDELSKNKVSVTCLVKDFYPTDIDIEWKSNGQPEPETKYSTTPA
QLDSDGSYFLYSKLTVETNRWQQGTTFTCAVMHEALHNHYTEKSVSKSPGK
HORSE_IGHG2 constant region (SEQ ID NO: 28)
CVLSAEGVIPIPSVPKPQCPPYTHSKFLGGPSVFIFPPNPKDALMISRTPVVTCVVVNLSDQ
YPDVQFSWYVDNTEVHSAITKQREAQFNSTYRVVSVLPIQHQDWLSGKEFKCSVTNVG
VPQPISRAISRGKGPSRVPQVYVLPPHPDELAKSKVSVTCLVKDFYPPDISVEWQSNRWP
ELEGKYSTTPAQLDGDGSYFLYSKLSLETSRWQQVESFTCAVMHEALHNHFTKTDISES
LGK
HORSE_IGHG3 constant region (SEQ ID NO: 29)
TTPPCPCECPKCPAPELLGGPSVFIFPPKPKDVLMITRTPEVTCLVVDVSHDSSDVLFTWY
VDGTEVKTAKTMPNEEQNNSTYRVVSVLRIQHQDWLNGKKFKCKVNNQALPAPVERTI
SKATGQTRVPQVYVLAPHPDELSKNKVSVTCLVKDFLPTDITVEWQSNEHPEPEGKYRT
TEAQKDSDGSYFLYSKLTVETDRWQQGTTFTCVVMHEALHNHVMQKNVSHSPGK
HORSE_IGHG4 constant region (SEQ ID NO: 30)
VIKECNGGCPAECLQVGPSVFIFPPKPKDVLMISRTPTVTCVVVDVGHDFPDVQFNWYV
DGVETHTATTEPKQEQFNSTYRVVSVLPIQHKDWLSGKEFKCKVNNKALPAPVERTISK
PTGQPREPQVYVLAPHRDELSKNKVSVTCLVKDFYPTDIDIEWKSNGQPEPETKYSTTPA
QLDSDGSYFLYSKLTVETNRWQQGTTFTCAVMHEALHNHYTEKSVSKSPGK
HORSE_IGHG5 constant region (SEQ ID NO: 31)
VVKGSPCPKCPAPELPGGPSVFIFPPKPKDVLKISRKPEVTCVVVDLGHDDPDVQFTWFV
DGVETHTATTEPKEEQFNSTYRVVSVLPIQHQDWLSGKEFKCSVTNKALPAPVERTTSK
AKGQLRVPQVYVLAPHPDELAKNTVSVTCLVKDFYPPEIDVEWQSNEHPEPEGKYSTTP
AQLNSDGSYFLYSKLSVETSRWKQGESFTCGVMHEAVENHYTQKNVSHSPGK
HORSE_IGHG6 constant region (SEQ ID NO: 32)
KEPCCCPKCPGRPSVFIFPPNPKDTLMISRTPEVTCVVVDVSQENPDVKFNWYVDGVEA
HTATTKAKEKQDNSTYRVVSVLPIQHQDWRRGKEFKCKVNNRALPAPVERTITKAKGE
LQDPKVYILAPHREEVTKNTVSVTCLVKDFYPPDINVEWQSNEEPEPEVKYSTTPAQLD
GDGSYFLYSKLTVETDRWEQGESFTCVVMHEAIRHTYRQKSITNFPGK
HORSE_IGHG7 constant region (SEQ ID NO: 33)
VIKECGGCPTCPECLSVGPSVFIFPPKPKDVLMISRTPTVTCVVVDVGHDFPDVQFNWYV
DGVETHTATTEPKQEQNNSTYRVVSILAIQHKDWLSGKEFKCKVNNQALPAPVQKTISK
PTGQPREPQVYVLAPHRDELSKNKVSVTCLVKDFYPTDIDIEWKSNGQPEPETKYSTTPA
QLDSDGSYFLYSKLTVETNRWQQGTTFTCAVMHEALHNHYTEKSVSKSPGK
Portion of canine ECD (without stalk and without alpha and gamma
secretase cleavage 3′ of the stalk region) that can be used in the
fusion constructs with mutations described herein (SEQ ID NO: 34)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVDPCLPCTVCEDTERQLRECTRWADAECEEIP
Tandem repeat of portion of canine ECD (without stalk and without alpha
and gamma secretase cleavage 3′ of the stalk region) that can be used
in the fusion constructs with mutations described herein (SEQ ID NO: 117)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVDPCLPCTVCEDTERQLRECTRWADAECEEIPKEACPTGLYTH
SGECCKACNLGEGVAQPCGANQTVCEPCLDSVTFSDVVSATEPCKPCTECVGLQSMSAP
CVEADDAVCRCAYGYYQDETTGRCEACRVCEAGSGLVFSCQDRQNTVCEECPDGTYS
DEANHVDPCLPCTVCEDTERQLRECTRWADAECEEIP
Canine p75NTR ECD nucleic acid sequence as used in certain fusion
constructs (encoding only the first two stalk amino acids GR and without
alpha and gamma secretase cleavage 3′ of the stalk region) (SEQ ID NO: 35)
AAGGAGGCATGTCCCACTGGCCTGTACACCCACAGCGGCGAGTGCTGCAAAGCCTG
CAATCTGGGTGAGGGGGTGGCCCAGCCTTGCGGAGCCAACCAGACCGTGTGTGAGC
CCTGCCTGGACAGCGTGACCTTCTCGGACGTGGTGAGCGCCACCGAGCCGTGCAAG
CCGTGCACCGAGTGCGTGGGGCTGCAGAGCATGTCGGCGCCGTGCGTGGAGGCGGA
CGACGCCGTGTGCCGCTGCGCCTACGGCTACTACCAGGACGAGACGACGGGCCGCT
GCGAGGCGTGCCGCGTGTGCGAGGCGGGCTCGGGGCTCGTGTTCTCGTGCCAGGAC
AGGCAGAACACCGTGTGCGAGGAGTGTCCCGACGGCACGTACTCCGACGAGGCCAA
CCACGTGGACCCGTGCCTGCCCTGCACCGTGTGCGAGGACACCGAGCGCCAGCTGC
GCGAGTGCACGCGCTGGGCCGACGCCGAGTGCGAGGAGATCCCTGGCCGT
Bovine p75 NTR protein (SEQ ID NO: 36)
KEACLTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRATPPE
GSDSTDPSTQEPEVPPEQDLVTSTVSDVVTTVMGSSQPVVTRGTADNLIPVYCSILAAVV
VGLVAYIAFKRWNSCKQNKQGANSRPVNQTPPPEGEKLHSDSGISVDSQSLHDQQPHTQ
TAAGQALKGDGGLYSSLPLAKREEVEKLLNGSAGDTWRHLAGELGYQPEHIDSFTHEA
CPARALLASWAAQDSATLDTLLAALRRIQRADLVESLCSESTATSPV
ECD is underlined.
Bovine p75 NTR nucleic acid (SEQ ID NO: 37)
ATGGGGTCAGGTGCCGCCGGCCGCGCCATGGACGGGCCGCGCCTGCTGCTGCTGCT
GCTGCTGCTCCTGGGGGTGTCCCTTGGAGGTGCCAAGGAAGCATGCCTCACGGGCCT
GTACACCCACAGCGGAGAGTGCTGCAAAGCCTGCAACCTGGGCGAGGGTGTGGCCC
AGCCTTGTGGAGCCAACCAGACCGTGTGTGAACCCTGCCTGGACAGCGTGACCTTCT
CGGACGTGGTGAGCGCCACGGAGCCGTGTAAGCCGTGCACGGAGTGCGTGGGACTG
CAGAGCATGTCGGCGCCCTGCGTGGAGGCCGACGACGCCGTGTGCCGCTGCGCCTA
CGGCTATTACCAGGACGAGACGACCGGCCGCTGCGAGGCGTGCCGCGTGTGCGAGG
CGGGCTCGGGGCTCGTGTTCTCGTGCCAGGACAAGCAGAACACCGTCTGCGAGGAG
TGCCCCGACGGCACGTACTCCGACGAGGCCAACCACGTGGACCCCTGCCTGCCCTGC
ACGGTGTGCGAGGACACGGAGCGCCAGCTGCGCGAGTGCACGCGCTGGGCCGACGC
CGAGTGCGAGGAGATCCCTGGACGTTGGATTACACGGGCCACGCCCCCTGAGGGCT
CCGACAGCACAGACCCCAGCACCCAGGAGCCCGAGGTACCTCCAGAGCAAGATCTG
GTAACCAGCACTGTGTCAGATGTGGTGACCACGGTGATGGGCAGCTCCCAGCCTGTG
GTGACCCGAGGTACCGCCGACAACCTCATCCCTGTCTATTGCTCCATCCTGGCTGCT
GTGGTTGTGGGCCTTGTGGCCTACATCGCCTTCAAGAGGTGGAACAGCTGCAAGCAG
AACAAGCAAGGAGCCAACAGCCGACCTGTGAACCAGACACCCCCACCAGAGGGGG
AAAAGCTACACAGCGATAGCGGCATCTCTGTGGACAGCCAGAGCCTGCATGACCAG
CAGCCCCACACGCAGACTGCCGCAGGCCAGGCCCTCAAGGGTGATGGAGGCCTCTA
CAGCAGCCTGCCGCTGGCCAAGCGGGAGGAGGTGGAGAAGCTGCTCAACGGCTCTG
CGGGGGACACCTGGCGGCATCTGGCAGGCGAGTTGGGTTACCAGCCTGAGCACATA
GACTCCTTCACCCACGAGGCCTGCCCAGCCCGCGCCCTGCTGGCCAGCTGGGCTGCC
CAGGACAGCGCCACGCTCGACACCCTCCTTGCGGCCCTGCGCCGCATCCAGCGCGCC
GACATCGTGGAGAGCCTGTGCAGCGAGTCCACGGCCACGTCCCCCGTGTGA
Feline p75NTR protein ECD (SEQ ID NO: 38)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVDPCLPCTVCEDTERQLRECTRWADAECEEIPGRWITRSTPSE
GSDSTAPSTEEPEVPPEQDLIASTVADVVTTVMGSSQPVVTRGTADN
Feline p75NTR ECD-feline IgG2 wt Fc protein fusion (SEQ ID NO: 39)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFSCQDRQNTVCEECPDGTYSDEANHVDPCLPCTVCEDTERQLRECT
RWADAECEEIPGRGGGGVPKTASTIESKTCDCPKCPVPEIPGAPSVFIFPPKPKDTLSISR
TPEVTCLVVDLGPDDSNVQITWFVDNTEMHTAKTRPREEQFNSTYRVVSVLPILHQDWL
KGKEFKCKVSKSLPSAMERTISKAKGQPHEPQVYVLPPTQEELSENKVSVTCLIKGFHPP
DIAVEWEITGQPEPENNYQTTPPQLDSDGTYFLYSRLSVDRSHWQRGNTYTCSVSHEAL
HSHHTQKSLTQSPGK
Signal peptide-Feline p75-ECD-Linker GGGG-Feline Fc-IgG2
Feline p75NTR ECD- feline IgG2 wt Fc nucleic acid sequence (SEQ ID NO: 40)
ATGGATGGACCTAGACCTCTGCTGCTGCTCCTGCCTCTGCTGTTGGGAGTTTCTCTCG
GCGGAGCCAAAGAGGCTTGTCCTACCGGCCTGTTTACCCACTCTGGCGAGTGTTGCA
AGGCCTGTAATCTCGGCGAAGGCGTGGCACAACCTTGTGGCGCTAATCAGACAGTG
TGCGAGCCTTGCCTGGACTCCGTGACCTTCTCTGATGTGGTGTCTGCCACCGAGCCA
TGCAAGCCTTGTACCGAGTGTGTGGGCCTGCAGTCCATGTCTGCCCCTTGTGTGGAA
GCCGACGACGCCGTGTGTAGATGTGCCTACGGCTACTACCAGGACGAGACAACCGG
AAGATGCGAGGCCTGCAGAGTGTGTGAAGCTGGCTCTGGACTGGTGTTCTCCTGCCA
AGACAGACAGAACACCGTGTGCGAGGAATGCCCTGACGGCACCTACTCTGATGAGG
CCAATCACGTGGACCCCTGCCTGCCTTGTACTGTGTGCGAAGATACCGAGCGGCAGC
TGCGCGAGTGTACCAGATGGGCTGATGCCGAGTGCGAAGAGATCCCTGGAAGAGGC
GGAGGCGGAGTGCCTAAGACCGCTTCTACCATCGAGTCCAAGACCTGCGACTGCCC
TAAGTGCCCTGTGCCTGAAATTCCTGGCGCTCCCTCCGTGTTCATCTTCCCACCTAAG
CCTAAGGACACCCTGTCCATCTCTCGGACCCCTGAAGTGACCTGCCTGGTGGTTGAT
CTGGGCCCTGACGACTCCAACGTGCAGATCACTTGGTTTGTGGACAACACCGAGATG
CACACCGCCAAGACCAGACCTAGAGAGGAACAGTTCAACTCCACCTACAGAGTGGT
GTCCGTGCTGCCCATCCTGCACCAGGATTGGCTGAAGGGCAAAGAATTCAAGTGCA
AAGTGAACTCCAAGAGCCTGCCTTCCGCCATGGAACGGACCATCTCTAAGGCTAAG
GGCCAGCCTCATGAGCCCCAGGTGTACGTTCTGCCTCCTACACAAGAGGAACTGTCC
GAGAACAAAGTGTCCGTGACATGCCTGATCAAGGGCTTTCACCCTCCTGATATCGCC
GTGGAATGGGAGATCACCGGACAGCCTGAGCCTGAGAACAACTACCAGACCACACC
TCCTCAGCTGGACAGCGACGGAACCTACTTCCTGTACTCCCGGCTGTCCGTGGACAG
ATCCCATTGGCAGAGAGGCAACACCTACACCTGTTCCGTGTCTCACGAGGCCCTGCA
CTCTCATCACACCCAGAAGTCCCTGACACAGTCCCCTGGCAAG
Feline IgG3 (SEQ ID NO: 41)
LPPCKCPKCPVPEIPGGPSVFIFPPKPKDTLSISRTPEVTCLVVDLGPDDSNVQITWFVDNT
EMHTAKTRPREEQFNSTYRVVSVLPIVHQDWLTGKEFKCKVNSKALPSAIERTISKAKG
QPHEPQVYVLPPAQEELSENKVCVTCLIKGFYPPDIAVEWEITGQPEPENNYRTTPPQLD
SDGTYFVYSRLSMDRSRWQSGNTYTCSVSHEALHSHHTQKSLTQSPGK
Feline p75NTR ECD-feline IgG1 wt Fc protein fusion (SEQ ID NO: 42)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFSCQDRQNTVCEECPDGTYSDEANHVDPCLPCTVCEDTERQLRECT
RWADAECEEIPGGGGVRKTDHPPGPKPCDCPKCPAPEMLGGPSIFIFPPKPKDTLSISRTP
EVTCLVVDLGPDDSDVQITWFVDNTQVYTAKTSPREEQFNSTYRVVSVLPILHQDWLK
GKEFKCKVNSKSLPSPIERTISKAKGQPHEPQVYVLPPAQEELSRNKVSVTCLIKSFHPPDI
AVEWEITGQPEPENNYRTTPPQLDSDGTYFVYSKLSVDRSHWQRGNTYTCSVSHEALHS
HHTQKSLTQSPGK
Signal peptide-Feline p75-ECD-Linker GGGG-Feline Fc-IgG1
Feline p75NTR ECD-feline Ig1 wt Fc nucleic acid sequence (SEQ ID NO: 43)
ATGGATGGCCCTAGACCTCTGCTGCTGCTGTTGCCTCTGCTCCTGGGAGTTTCTCTCG
GCGGAGCCAAAGAGGCTTGTCCTACCGGCCTGTTTACCCACTCTGGCGAGTGTTGCA
AGGCCTGTAATCTCGGCGAAGGCGTGGCACAACCTTGTGGCGCTAATCAGACAGTG
TGCGAGCCTTGCCTGGACTCCGTGACCTTCTCTGATGTGGTGTCTGCCACCGAGCCA
TGCAAGCCTTGTACCGAGTGTGTGGGCCTGCAGTCCATGTCTGCCCCTTGTGTGGAA
GCCGACGACGCCGTGTGTAGATGTGCCTACGGCTACTACCAGGACGAGACAACCGG
AAGATGCGAGGCCTGCAGAGTGTGTGAAGCTGGCTCTGGACTGGTGTTCTCCTGCCA
AGACAGACAGAACACCGTGTGCGAGGAATGCCCTGACGGCACCTACTCTGATGAGG
CCAATCACGTGGACCCCTGCCTGCCTTGTACTGTGTGCGAAGATACCGAGCGGCAGC
TGCGCGAGTGTACCAGATGGGCTGATGCCGAGTGCGAAGAGATTCCTGGCGGAGGC
GGAGTGCGCAAGACAGATCATCCTCCTGGACCTAAGCCTTGCGACTGCCCTAAGTGT
CCCGCTCCTGAAATGCTCGGCGGACCCAGCATCTTCATCTTCCCACCTAAGCCAAAG
GACACCCTGTCCATCTCTCGGACCCCTGAAGTGACCTGCCTGGTGGTTGATCTGGGC
CCTGACGATTCCGACGTGCAGATCACTTGGTTTGTGGACAACACCCAGGTGTACACA
GCCAAGACCTCTCCAAGAGAGGAACAGTTCAACTCCACCTACAGAGTGGTGTCCGT
GCTGCCCATCCTGCACCAGGATTGGCTGAAGGGCAAAGAATTCAAGTGCAAAGTGA
ACTCCAAGAGCCTGCCTTCTCCAATCGAGCGGACCATCTCCAAGGCTAAGGGCCAG
CCTCATGAGCCTCAGGTGTACGTTCTGCCTCCTGCTCAAGAGGAACTGTCCCGGAAC
AAAGTGTCTGTGACCTGTCTGATCAAGAGCTTTCACCCTCCTGATATCGCCGTGGAA
TGGGAGATCACCGGACAGCCTGAGCCTGAGAACAACTACCGGACCACACCTCCTCA
GCTGGACAGCGACGGCACATACTTCGTGTACTCCAAGCTGTCCGTGGACAGATCCCA
CTGGCAGCGGGGCAATACCTACACCTGTTCCGTGTCTCACGAGGCCCTGCACTCTCA
TCACACCCAGAAGTCCCTGACACAGTCCCCTGGAAAGTGATGA
Feline p75NTR ECD-feline IgG3 wt Fc protein fusion (SEQ ID NO: 44)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFSCQDRQNTVCEECPDGTYSDEANHVDPCLPCTVCEDTERQLRECT
RWADAECEEIPGGGGVLPPCKCPKCPVPEIPGGPSVFIFPPKPKDTLSISRTPEVTCLVVD
LGPDDSNVQITWFVDNTEMHTAKTRPREEQFNSTYRVVSVLPIVHQDWLTGKEFKCKV
NSKALPSAIERTISKAKGQPHEPQVYVLPPAQEELSENKVCVTCLIKGFYPPDIAVEWEIT
GQPEPENNYRTTPPQLDSDGTYFVYSRLSMDRSRWQSGNTYTCSVSHEALHSHHTQKSL
TQSPGK
Signal peptide-Feline p75-ECD-Linker GGGG-Feline Fc-IgG3
Feline p75NTR ECD-feline IgG3 wt Fc nucleic acid sequence (SEQ ID NO: 45)
ATGGATGGCCCTAGACCTCTGCTGCTGCTGTTGCCTCTGCTCCTGGGAGTTTCTCTCG
GCGGAGCCAAAGAGGCTTGTCCTACCGGCCTGTTTACCCACTCTGGCGAGTGTTGCA
AGGCCTGTAATCTCGGCGAAGGCGTGGCACAACCTTGTGGCGCTAATCAGACAGTG
TGCGAGCCTTGCCTGGACTCCGTGACCTTCTCTGATGTGGTGTCTGCCACCGAGCCA
TGCAAGCCTTGTACCGAGTGTGTGGGCCTGCAGTCCATGTCTGCCCCTTGTGTGGAA
GCCGACGACGCCGTGTGTAGATGTGCCTACGGCTACTACCAGGACGAGACAACCGG
AAGATGCGAGGCCTGCAGAGTGTGTGAAGCTGGCTCTGGACTGGTGTTCTCCTGCCA
AGACAGACAGAACACCGTGTGCGAGGAATGCCCTGACGGCACCTACTCTGATGAGG
CCAATCACGTGGACCCCTGCCTGCCTTGTACTGTGTGCGAAGATACCGAGCGGCAGC
TGCGCGAGTGTACCAGATGGGCTGATGCCGAGTGCGAAGAGATTCCTGGCGGAGGC
GGAGTTCTGCCTCCTTGCAAGTGTCCTAAGTGCCCCGTGCCTGAAATCCCTGGCGGC
CCTTCCGTGTTCATCTTCCCACCTAAGCCTAAGGACACCCTGTCCATCTCTCGGACCC
CTGAAGTGACCTGCCTGGTGGTTGATCTGGGCCCTGACGACTCCAACGTGCAGATCA
CTTGGTTTGTGGACAACACCGAGATGCACACCGCCAAGACCAGACCTAGAGAGGAA
CAGTTCAACTCCACCTACAGAGTGGTGTCCGTGCTGCCCATCGTGCACCAGGATTGG
CTGACCGGCAAAGAATTCAAGTGCAAAGTGAACAGCAAGGCCCTGCCTTCCGCCAT
CGAGCGGACAATCTCTAAGGCTAAGGGCCAGCCTCACGAGCCCCAGGTTTACGTTTT
GCCTCCTGCTCAAGAGGAACTGTCCGAGAACAAAGTGTGCGTGACCTGTCTGATCAA
GGGCTTCTACCCTCCTGATATCGCCGTGGAATGGGAGATCACCGGACAGCCTGAGCC
TGAGAACAACTACCGGACCACACCTCCTCAGCTGGATTCCGACGGCACATACTTCGT
GTACTCCCGGCTGAGCATGGACAGATCCAGATGGCAGTCCGGCAACACCTACACCT
GTTCCGTGTCTCACGAGGCCCTGCACTCTCATCACACCCAGAAGTCCCTGACACAGT
CCCCTGGCAAG
Variants
Canine p75NTR ECD E75T (SEQ ID NO: 46)
KEACPTGLYTHSGECCKACNLGEGVAQPCGANQTVCEPCLDSVTFSDVVSATEPCKPCT
ECVGLQSMSAPCVEATDAVCRCAYGYYQDETTGRCEACRVCEAGSGLVFSCQDRQNT
VCEECPDGTYSDEANHVDPCLPCTVCEDTERQLRECTRWADAECEEIPGR
Canine p75NTR ECD E75T-canine IgGB wt Fc (SEQ ID NO: 47)
MEWSWVFLFFLSVTTGVHSKEACPTGLYTHSGECCKACNLGEGVAQPCGANQTVCEPC
LDSVTFSDVVSATEPCKPCTECVGLQSMSAPCVEATDAVCRCAYGYYQDETTGRCEAC
RVCEAGSGLVFSCQDRQNTVCEECPDGTYSDEANHVDPCLPCTVCEDTERQLRECTRW
ADAECEEIPGRGGGGRENGRVPRPPDCPKCPAPEMLGGPSVFIFPPKPKDTLLIARTPEV
TCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDV
EWQSNGQQEPESKYRTTPPQLDEDGSYFLYSKLSVDKSRWQRGDTFICAVMHEALHNH
YTQKSLSHSPGK
Signal peptide-Canine p75-ECD-Linker GGGG-Canine Fc-B wt
Canine p75NTR ECD E75T-canine IgGB wt Fc nucleic acid sequence (SEQ
ID NO: 48)
ATGGAATGGAGCTGGGTGTTTCTGTTTTTTCTGAGCGTGACCACCGGCGTGCATAGC
AAAGAAGCGTGCCCGACCGGCCTGTATACCCATAGCGGCGAATGCTGCAAAGCGTG
CAACCTGGGCGAAGGCGTGGCGCAGCCGTGCGGCGCGAACCAGACCGTGTGCGAAC
CGTGCCTGGATAGCGTGACCTTTAGCGATGTGGTGAGCGCGACCGAACCGTGCAAA
CCGTGCACCGAATGCGTGGGCCTGCAGAGCATGAGCGCGCCGTGCGTGGAAGCGAC
CGATGCGGTGTGCCGCTGCGCGTATGGCTATTATCAGGATGAAACCACCGGCCGCTG
CGAAGCGTGCCGCGTGTGCGAAGCGGGCAGCGGCCTGGTGTTTAGCTGCCAGGATC
GCCAGAACACCGTGTGCGAAGAATGCCCGGATGGCACCTATAGCGATGAAGCGAAC
CATGTGGATCCGTGCCTGCCGTGCACCGTGTGCGAAGATACCGAACGCCAGCTGCG
CGAATGCACCCGCTGGGCGGATGCGGAATGCGAAGAAATTCCGGGCCGCGGCGGCG
GCGGCCGCGAAAACGGCCGCGTGCCGCGCCCGCCGGATTGCCCGAAATGCCCGGCG
CCGGAAATGCTGGGCGGCCCGAGCGTGTTTATTTTTCCGCCGAAACCGAAAGATACC
CTGCTGATTGCGCGCACCCCGGAAGTGACCTGCGTGGTGGTGGATCTGGATCCGGAA
GATCCGGAAGTGCAGATTAGCTGGTTTGTGGATGGCAAACAGATGCAGACCGCGAA
AACCCAGCCGCGCGAAGAACAGTTTAACGGCACCTATCGCGTGGTGAGCGTGCTGC
CGATTGGCCATCAGGATTGGCTGAAAGGCAAACAGTTTACCTGCAAAGTGAACAAC
AAAGCGCTGCCGAGCCCGATTGAACGCACCATTAGCAAAGCGCGCGGCCAGGCGCA
TCAGCCGAGCGTGTATGTGCTGCCGCCGAGCCGCGAAGAACTGAGCAAAAACACCG
TGAGCCTGACCTGCCTGATTAAAGATTTTTTTCCGCCGGATATTGATGTGGAATGGC
AGAGCAACGGCCAGCAGGAACCGGAAAGCAAATATCGCACCACCCCGCCGCAGCT
GGATGAAGATGGCAGCTATTTTCTGTATAGCAAACTGAGCGTGGATAAAAGCCGCT
GGCAGCGCGGCGATACCTTTATTTGCGCGGTGATGCATGAAGCGCTGCATAACCATT
ATACCCAGAAAAGCCTGAGCCATAGCCCGGGCAAA
Canine p75NTR ECD S109Y (SEQ ID NO: 49)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFYCQDRQNT
VCEECPDGTYSDEANHVDPCLPCTVCEDTERQLRECTRWADAECEEIPGR
Canine p75NTR ECD S109Y-canine IgGB wt Fc (SEQ ID NO: 50)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFYCQDRQNTVCEECPDGTYSDEANHVDPCLPCTVCEDTERQLRECTRW
ADAECEEIPGRGGGGRENGRVPRPPDCPKCPAPEMLGGPSVFIFPPKPKDTLLIARTPEV
TCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDV
EWQSNGQQEPESKYRTTPPQLDEDGSYFLYSKLSVDKSRWQRGDTFICAVMHEALHINH
YTQKSLSHSPGK
Signal peptide-Canine p75-ECD-Linker GGGG-Canine Fc-B wt
Canine p75NTR ECD S109Y-canine IgGB wt Fc nucleic acid sequence (SEQ
ID NO: 51)
ATGGAATGGAGCTGGGTGTTTCTGTTTTTTCTGAGCGTGACCACCGGCGTGCATAGC
AAAGAAGCGTGCCCGACCGGCCTGTATACCCATAGCGGCGAATGCTGCAAAGCGTG
CAACCTGGGCGAAGGCGTGGCGCAGCCGTGCGGCGCGAACCAGACCGTGTGCGAAC
CGTGCCTGGATAGCGTGACCTTTAGCGATGTGGTGAGCGCGACCGAACCGTGCAAA
CCGTGCACCGAATGCGTGGGCCTGCAGAGCATGAGCGCGCCGTGCGTGGAAGCGGA
TGATGCGGTGTGCCGCTGCGCGTATGGCTATTATCAGGATGAAACCACCGGCCGCTG
CGAAGCGTGCCGCGTGTGCGAAGCGGGCAGCGGCCTGGTGTTTTATTGCCAGGATC
GCCAGAACACCGTGTGCGAAGAATGCCCGGATGGCACCTATAGCGATGAAGCGAAC
CATGTGGATCCGTGCCTGCCGTGCACCGTGTGCGAAGATACCGAACGCCAGCTGCG
CGAATGCACCCGCTGGGCGGATGCGGAATGCGAAGAAATTCCGGGCCGCGGCGGCG
GCGGCCGCGAAAACGGCCGCGTGCCGCGCCCGCCGGATTGCCCGAAATGCCCGGCG
CCGGAAATGCTGGGCGGCCCGAGCGTGTTTATTTTTCCGCCGAAACCGAAAGATACC
CTGCTGATTGCGCGCACCCCGGAAGTGACCTGCGTGGTGGTGGATCTGGATCCGGAA
GATCCGGAAGTGCAGATTAGCTGGTTTGTGGATGGCAAACAGATGCAGACCGCGAA
AACCCAGCCGCGCGAAGAACAGTTTAACGGCACCTATCGCGTGGTGAGCGTGCTGC
CGATTGGCCATCAGGATTGGCTGAAAGGCAAACAGTTTACCTGCAAAGTGAACAAC
AAAGCGCTGCCGAGCCCGATTGAACGCACCATTAGCAAAGCGCGCGGCCAGGCGCA
TCAGCCGAGCGTGTATGTGCTGCCGCCGAGCCGCGAAGAACTGAGCAAAAACACCG
TGAGCCTGACCTGCCTGATTAAAGATTTTTTTCCGCCGGATATTGATGTGGAATGGC
AGAGCAACGGCCAGCAGGAACCGGAAAGCAAATATCGCACCACCCCGCCGCAGCT
GGATGAAGATGGCAGCTATTTTCTGTATAGCAAACTGAGCGTGGATAAAAGCCGCT
GGCAGCGCGGCGATACCTTTATTTGCGCGGTGATGCATGAAGCGCTGCATAACCATT
ATACCCAGAAAAGCCTGAGCCATAGCCCGGGCAAA
Canine p75NTR ECD S109H (SEQ ID NO: 52)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFHCQDRQNT
VCEECPDGTYSDEANHVDPCLPCTVCEDTERQLRECTRWADAECEEIPGR
Canine p75NTR ECD S109H-canine IgGB wt Fc (SEQ ID NO: 53)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFHCQDRQNTVCEECPDGTYSDEANHVDPCLPCTVCEDTERQLRECTRW
ADAECEEIPGRGGGGRENGRVPRPPDCPKCPAPEMLGGPSVFIFPPKPKDTLLIARTPEV
TCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDV
EWQSNGQQEPESKYRTTPPQLDEDGSYFLYSKLSVDKSRWQRGDTFICAVMHEALHNH
YTQKSLSHSPGK
Signal peptide-Canine p75-ECD-Linker GGGG-Canine Fc-B wt
Canine p75NTR ECD S109H-canine IgGB wt Fc nucleic acid sequence (SEQ
ID NO: 54)
ATGGAATGGAGCTGGGTGTTTCTGTTTTTTCTGAGCGTGACCACCGGCGTGCATAGC
AAAGAAGCGTGCCCGACCGGCCTGTATACCCATAGCGGCGAATGCTGCAAAGCGTG
CAACCTGGGCGAAGGCGTGGCGCAGCCGTGCGGCGCGAACCAGACCGTGTGCGAAC
CGTGCCTGGATAGCGTGACCTTTAGCGATGTGGTGAGCGCGACCGAACCGTGCAAA
CCGTGCACCGAATGCGTGGGCCTGCAGAGCATGAGCGCGCCGTGCGTGGAAGCGGA
TGATGCGGTGTGCCGCTGCGCGTATGGCTATTATCAGGATGAAACCACCGGCCGCTG
CGAAGCGTGCCGCGTGTGCGAAGCGGGCAGCGGCCTGGTGTTTCATTGCCAGGATC
GCCAGAACACCGTGTGCGAAGAATGCCCGGATGGCACCTATAGCGATGAAGCGAAC
CATGTGGATCCGTGCCTGCCGTGCACCGTGTGCGAAGATACCGAACGCCAGCTGCG
CGAATGCACCCGCTGGGCGGATGCGGAATGCGAAGAAATTCCGGGCCGCGGCGGCG
GCGGCCGCGAAAACGGCCGCGTGCCGCGCCCGCCGGATTGCCCGAAATGCCCGGCG
CCGGAAATGCTGGGCGGCCCGAGCGTGTTTATTTTTCCGCCGAAACCGAAAGATACC
CTGCTGATTGCGCGCACCCCGGAAGTGACCTGCGTGGTGGTGGATCTGGATCCGGAA
GATCCGGAAGTGCAGATTAGCTGGTTTGTGGATGGCAAACAGATGCAGACCGCGAA
AACCCAGCCGCGCGAAGAACAGTTTAACGGCACCTATCGCGTGGTGAGCGTGCTGC
CGATTGGCCATCAGGATTGGCTGAAAGGCAAACAGTTTACCTGCAAAGTGAACAAC
AAAGCGCTGCCGAGCCCGATTGAACGCACCATTAGCAAAGCGCGCGGCCAGGCGCA
TCAGCCGAGCGTGTATGTGCTGCCGCCGAGCCGCGAAGAACTGAGCAAAAACACCG
TGAGCCTGACCTGCCTGATTAAAGATTTTTTTCCGCCGGATATTGATGTGGAATGGC
AGAGCAACGGCCAGCAGGAACCGGAAAGCAAATATCGCACCACCCCGCCGCAGCT
GGATGAAGATGGCAGCTATTTTCTGTATAGCAAACTGAGCGTGGATAAAAGCCGCT
GGCAGCGCGGCGATACCTTTATTTGCGCGGTGATGCATGAAGCGCTGCATAACCATT
ATACCCAGAAAAGCCTGAGCCATAGCCCGGGCAAA
Canine p75NTR ECD V133R (SEQ ID NO: 55)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHRDPCLPCTVCEDTERQLRECTRWADAECEEIPGR
Canine p75NTR ECD V133R-canine IgGB wt Fc (SEQ ID NO: 56)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFSCQDRQNTVCEECPDGTYSDEANHRDPCLPCTVCEDTERQLRECTRW
ADAECEEIPGRGGGGRENGRVPRPPDCPKCPAPEMLGGPSVFIFPPKPKDTLLIARTPEV
TCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDV
EWQSNGQQEPESKYRTTPPQLDEDGSYFLYSKLSVDKSRWQRGDTFICAVMHEALHNH
YTQKSLSHSPGK
Signal peptide-Canine p75-ECD-Linker GGGG-Canine Fc-B wt
Canine p75NTR ECD V133R-canine IgGB wt Fc nucleic acid sequence (SEQ
ID NO: 57)
ATGGAATGGAGCTGGGTGTTTCTGTTTTTTCTGAGCGTGACCACCGGCGTGCATAGC
AAAGAAGCGTGCCCGACCGGCCTGTATACCCATAGCGGCGAATGCTGCAAAGCGTG
CAACCTGGGCGAAGGCGTGGCGCAGCCGTGCGGCGCGAACCAGACCGTGTGCGAAC
CGTGCCTGGATAGCGTGACCTTTAGCGATGTGGTGAGCGCGACCGAACCGTGCAAA
CCGTGCACCGAATGCGTGGGCCTGCAGAGCATGAGCGCGCCGTGCGTGGAAGCGGA
TGATGCGGTGTGCCGCTGCGCGTATGGCTATTATCAGGATGAAACCACCGGCCGCTG
CGAAGCGTGCCGCGTGTGCGAAGCGGGCAGCGGCCTGGTGTTTAGCTGCCAGGATC
GCCAGAACACCGTGTGCGAAGAATGCCCGGATGGCACCTATAGCGATGAAGCGAAC
CATCGCGATCCGTGCCTGCCGTGCACCGTGTGCGAAGATACCGAACGCCAGCTGCG
CGAATGCACCCGCTGGGCGGATGCGGAATGCGAAGAAATTCCGGGCCGCGGCGGCG
GCGGCCGCGAAAACGGCCGCGTGCCGCGCCCGCCGGATTGCCCGAAATGCCCGGCG
CCGGAAATGCTGGGCGGCCCGAGCGTGTTTATTTTTCCGCCGAAACCGAAAGATACC
CTGCTGATTGCGCGCACCCCGGAAGTGACCTGCGTGGTGGTGGATCTGGATCCGGAA
GATCCGGAAGTGCAGATTAGCTGGTTTGTGGATGGCAAACAGATGCAGACCGCGAA
AACCCAGCCGCGCGAAGAACAGTTTAACGGCACCTATCGCGTGGTGAGCGTGCTGC
CGATTGGCCATCAGGATTGGCTGAAAGGCAAACAGTTTACCTGCAAAGTGAACAAC
AAAGCGCTGCCGAGCCCGATTGAACGCACCATTAGCAAAGCGCGCGGCCAGGCGCA
TCAGCCGAGCGTGTATGTGCTGCCGCCGAGCCGCGAAGAACTGAGCAAAAACACCG
TGAGCCTGACCTGCCTGATTAAAGATTTTTTTCCGCCGGATATTGATGTGGAATGGC
AGAGCAACGGCCAGCAGGAACCGGAAAGCAAATATCGCACCACCCCGCCGCAGCT
GGATGAAGATGGCAGCTATTTTCTGTATAGCAAACTGAGCGTGGATAAAAGCCGCT
GGCAGCGCGGCGATACCTTTATTTGCGCGGTGATGCATGAAGCGCTGCATAACCATT
ATACCCAGAAAAGCCTGAGCCATAGCCCGGGCAAA
Canine p75NTR ECD D134L (SEQ ID NO: 58)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVLPCLPCTVCEDTERQLRECTRWADAECEEIPGR
Canine p75NTR ECD D134L-canine IgGB wt Fc (SEQ ID NO: 59)
MEWSWVFLFFLSVTTGVHSKEACPTGLYTHSGECCKACNLGEGVAQPCGANQTVCEPC
LDSVTFSDVVSATEPCKPCTECVGLQSMSAPCVEADDAVCRCAYGYYQDETTGRCEAC
RVCEAGSGLVFSCQDRQNTVCEECPDGTYSDEANHVLPCLPCTVCEDTERQLRECTRW
ADAECEEIPGRGGGGRENGRVPRPPDCPKCPAPEMLGGPSVFIFPPKPKDTLLIARTPEV
TCVVVDLDPEDPEVQISWFVDGKQMQTAKTQPREEQFNGTYRVVSVLPIGHQDWLKGK
QFTCKVNNKALPSPIERTISKARGQAHQPSVYVLPPSREELSKNTVSLTCLIKDFFPPDIDV
EWQSNGQQEPESKYRTTPPQLDEDGSYFLYSKLSVDKSRWQRGDTFICAVMHEALHNH
YTQKSLSHSPGK
Signal peptide-Canine p75-ECD-Linker GGGG-Canine Fc-B wt
Canine p75NTR ECD D134L-canine IgGB wt Fc nucleic acid sequence (SEQ
ID NO: 60)
ATGGAATGGAGCTGGGTGTTTCTGTTTTTTCTGAGCGTGACCACCGGCGTGCATAGC
AAAGAAGCGTGCCCGACCGGCCTGTATACCCATAGCGGCGAATGCTGCAAAGCGTG
CAACCTGGGCGAAGGCGTGGCGCAGCCGTGCGGCGCGAACCAGACCGTGTGCGAAC
CGTGCCTGGATAGCGTGACCTTTAGCGATGTGGTGAGCGCGACCGAACCGTGCAAA
CCGTGCACCGAATCGTGGGCCTGCAGAGCATGAGCGCGCCGTGCGTGGAAGCGGAT
GATGCGGTGTGCCGCTGCGCGTATGGCTATTATCAGGATGAAACCACCGGCCGCTGC
GAAGCGTGCCGCGTGTGCGAAGCGGGCAGCGGCCTGGTGTTTAGCTGCCAGGATCG
CCAGAACACCGTGTGCGAAGAATGCCCGGATGGCACCTATAGCGATGAAGCGAACC
ATGTGCTGCCGTGCCTGCCGTGCACCGTGTGCGAAGATACCGAACGCCAGCTGCGC
GAATGCACCCGCTGGGCGGATGCGGAATGCGAAGAAATTCCGGGCCGCGGCGGCGG
CGGCCGCGAAAACGGCCGCGTGCCGCGCCCGCCGGATTGCCCGAAATGCCCGGCGC
CGGAAATGCTGGGCGGCCCGAGCGTGTTTATTTTTCCGCCGAAACCGAAAGATACCC
TGCTGATTGCGCGCACCCCGGAAGTGACCTGCGTGGTGGTGGATCTGGATCCGGAA
GATCCGGAAGTGCAGATTAGCTGGTTTGTGGATGGCAAACAGATGCAGACCGCGAA
AACCCAGCCGCGCGAAGAACAGTTTAACGGCACCTATCGCGTGGTGAGCGTGCTGC
CGATTGGCCATCAGGATTGGCTGAAAGGCAAACAGTTTACCTGCAAAGTGAACAAC
AAAGCGCTGCCGAGCCCGATTGAACGCACCATTAGCAAAGCGCGCGGCCAGGCGCA
TCAGCCGAGCGTGTATGTGCTGCCGCCGAGCCGCGAAGAACTGAGCAAAAACACCG
TGAGCCTGACCTGCCTGATTAAAGATTTTTTTCCGCCGGATATTGATGTGGAATGGC
AGAGCAACGGCCAGCAGGAACCGGAAAGCAAATATCGCACCACCCCGCCGCAGCT
GGATGAAGATGGCAGCTATTTTCTGTATAGCAAACTGAGCGTGGATAAAAGCCGCT
GGCAGCGCGGCGATACCTTTATTTGCGCGGTGATGCATGAAGCGCTGCATAACCATT
ATACCCAGAAAAGCCTGAGCCATAGCCCGGGCAAA
Feline p75NTR ECD E75T (SEQ ID NO: 61)
KEACPTGLFTHSGECCKACNLGEGVAQPCGANQTVCEPCLDSVTFSDVVSATEPCKPCT
ECVGLQSMSAPCVEATDAVCRCAYGYYQDETTGRCEACRVCEAGSGLVFSCQDRQNT
VCEECPDGTYSDEANHVDPCLPCTVCEDTERQLRECTRWADAECEEIP
Feline p75NTR ECD E75T-Feline IgG1 (SEQ ID NO: 62)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEATDAVCRCAYGYYQDETTGRC
EACRVCEAGSGLVFSCQDRQNTVCEECPDGTYSDEANHVDPCLPCTVCEDTERQLRECT
RWADAECEEIPGGGGVRKTDHPPGPKPCDCPKCPAPEMLGGPSIFIFPPKPKDTLSISRTP
EVTCLVVDLGPDDSDVQITWFVDNTQVYTAKTSPREEQFNSTYRVVSVLPILHQDWLK
GKEFKCKVNSKSLPSPIERTISKAKGQPHEPQVYVLPPAQEELSRNKVSVTCLIKSFHPPDI
AVEWEITGQPEPENNYRTTPPQLDSDGTYFVYSKLSVDRSHWQRGNTYTCSVSHEALHS
HHTQKSLTQSPGK
Signal peptide-Feline p75-ECD-Linker GGGG-Feline Fc-IgG1
Feline p75NTR ECD S109Y (SEQ ID NO: 63)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFYCQDRQNT
VCEECPDGTYSDEANHVDPCLPCTVCEDTERQLRECTRWADAECEEIP
Feline p75NTR ECD S109Y-Feline Fc IgG1 (SEQ ID NO: 64)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFYCQDRQNTVCEECPDGTYSDEANHVDPCLPCTVCEDTERQLREC
TRWADAECEEIPGGGGVRKTDHPPGPKPCDCPKCPAPEMLGGPSIFIFPPKPKDTLSISR
TPEVTCLVVDLGPDDSDVQITWFVDNTQVYTAKTSPREEQFNSTYRVVSVLPILHQDWL
KGKEFKCKVNSKSLPSPIERTISKAKGQPHEPQVYVLPPAQEELSRNKVSVTCLIKSFHPP
DIAVEWEITGQPEPENNYRTTPPQLDSDGTYFVYSKLSVDRSHWQRGNTYTCSVSHEAL
HSHHTQKSLTQSPGK
Signal peptide-Feline p75-ECD-Linker GGGG-Feline Fc-IgG1
Feline p75NTR ECD S109H (SEQ ID NO: 65)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFHCQDRQNT
VCEECPDGTYSDEANHVDPCLPCTVCEDTERQLRECTRWADAECEEIP
Feline p75NTR ECD S109H-Feline Fc IgG1 (SEQ ID NO: 66)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFHCQDRQNTVCEECPDGTYSDEANHVDPCLPCTVCEDTERQLREC
TRWADAECEEIPGGGGVRKTDHPPGPKPCDCPKCPAPEMLGGPSIFIFPPKPKDTLSISR
TPEVTCLVVDLGPDDSDVQITWFVDNTQVYTAKTSPREEQFNSTYRVVSVLPILHQDWL
KGKEFKCKVNSKSLPSPIERTISKAKGQPHEPQVYVLPPAQEELSRNKVSVTCLIKSFHPP
DIAVEWEITGQPEPENNYRTTPPQLDSDGTYFVYSKLSVDRSHWQRGNTYTCSVSHEAL
HSHHTQKSLTQSPGK
Signal peptide-Feline p75-ECD-Linker GGGG-Feline Fc-IgG1
Feline p75NTR ECD V133R (SEQ ID NO: 67)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHRDPCLPCTVCEDTERQLRECTRWADAECEEIP
Feline p75NTR ECD V133R-Feline Fc IgG1 (SEQ ID NO: 68)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFSCQDRQNTVCEECPDGTYSDEANHRDPCLPCTVCEDTERQLREC
TRWADAECEEIPGGGGVRKTDHPPGPKPCDCPKCPAPEMLGGPSIFIFPPKPKDTLSISR
TPEVTCLVVDLGPDDSDVQITWFVDNTQVYTAKTSPREEQFNSTYRVVSVLPILHQDWL
KGKEFKCKVNSKSLPSPIERTISKAKGQPHEPQVYVLPPAQEELSRNKVSVTCLIKSFHPP
DIAVEWEITGQPEPENNYRTTPPQLDSDGTYFVYSKLSVDRSHWQRGNTYTCSVSHEAL
HSHHTQKSLTQSPGK
Signal peptide-Feline p75-ECD-Linker GGGG-Feline Fc-IgG1
Feline p75NTR ECD D134L (SEQ ID NO: 69)
KEACPTGLFTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDRQNT
VCEECPDGTYSDEANHVLPCLPCTVCEDTERQLRECTRWADAECEEIP
Feline p75NTR ECD D134L-Feline Fc IgG1 (SEQ ID NO: 70)
MDGPRPLLLLLPLLLGVSLGGAKEACPTGLFTHSGECCKACNLGEGVAQPCGANQTVC
EPCLDSVTFSDVVSATEPCKPCTECVGLQSMSAPCVEADDAVCRCAYGYYQDETTGRC
EACRVCEAGSGLVFSCQDRQNTVCEECPDGTYSDEANHVLPCLPCTVCEDTERQLREC
TRWADAECEEIPGGGGVRKTDHPPGPKPCDCPKCPAPEMLGGPSIFIFPPKPKDTLSISR
TPEVTCLVVDLGPDDSDVQITWFVDNTQVYTAKTSPREEQFNSTYRVVSVLPILHQDWL
KGKEFKCKVNSKSLPSPIERTISKAKGQPHEPQVYVLPPAQEELSRNKVSVTCLIKSFHPP
DIAVEWEITGQPEPENNYRTTPPQLDSDGTYFVYSKLSVDRSHWQRGNTYTCSVSHEAL
HSHHTQKSLTQSPGK
Signal peptide-Feline p75-ECD-Linker GGGG-Feline Fc-IgG1
Human p75NTR protein (SEQ ID NO: 71)
>sp|P08138|TNR16_HUMAN Tumor necrosis factor receptor superfamily member 16
OS = Homo sapiens OX = 9606 GN = NGFR PE = 1 SV = 1
MGAGATGRAMDGPRLLLLLLLGVSLGGAKEACPTGLYTHSGECCKACNLGEGVAQPC
GANQTVCEPCLDSVTFSDVVSATEPCKPCTECVGLQSMSAPCVEADDAVCRCAYGYYQ
DETTGRCEACRVCEAGSGLVFSCQDKQNTVCEECPDGTYSDEANHVDPCLPCTVCEDT
ERQLRECTRWADAECEEIPGRWITRSTPPEGSDSTAPSTQEPEAPPEQDLIASTVAGVVTT
VMGSSQPVVTRGTTDNLIPVYCSILAAVVVGLVAYIAFKRWNSCKQNKQGANSRPVNQ
TPPPEGEKLHSDSGISVDSQSLHDQQPHTQTASGQALKGDGGLYSSLPPAKREEVEKLLN
GSAGDTWRHLAGELGYQPEHIDSFTHEACPVRALLASWATQDSATLDALLAALRRIQR
ADLVESLCSESTATSPV
Full p75NTR aa sequence including signal peptide, ECD, transmembrane and
intracellular domains
Human p75NTR nucleic acid sequence (SEQ ID NO: 72)
ATGGGCGCCGGCGCCACCGGCAGGGCCATGGACGGCCCCAGGCTGCTGCTGCTGCT
GCTGCTGGGCGTGAGCCTGGGCGGCGCCAAGGAGGCCTGCCCCACCGGCCTGTACA
CCCACAGCGGCGAGTGCTGCAAGGCCTGCAACCTGGGCGAGGGCGTGGCCCAGCCC
TGCGGCGCCAACCAGACCGTGTGCGAGCCCTGCCTGGACAGCGTGACCTTCAGCGA
CGTGGTGAGCGCCACCGAGCCCTGCAAGCCCTGCACCGAGTGCGTGGGCCTGCAGA
GCATGAGCGCCCCCTGCGTGGAGGCCGACGACGCCGTGTGCAGGTGCGCCTACGGC
TACTACCAGGACGAGACCACCGGCAGGTGCGAGGCCTGCAGGGTGTGCGAGGCCGG
CAGCGGCCTGGTGTTCAGCTGCCAGGACAAGCAGAACACCGTGTGCGAGGAGTGCC
CCGACGGCACCTACAGCGACGAGGCCAACCACGTGGACCCCTGCCTGCCCTGCACC
GTGTGCGAGGACACCGAGAGGCAGCTGAGGGAGTGCACCAGGTGGGCCGACGCCG
AGTGCGAGGAGATCCCCGGCAGGTGGATCACCAGGAGCACCCCCCCCGAGGGCAGC
GACAGCACCGCCCCCAGCACCCAGGAGCCCGAGGCCCCCCCCGAGCAGGACCTGAT
CGCCAGCACCGTGGCCGGCGTGGTGACCACCGTGATGGGCAGCAGCCAGCCCGTGG
TGACCAGGGGCACCACCGACAACCTGATCCCCGTGTACTGCAGCATCCTGGCCGCC
GTGGTGGTGGGCCTGGTGGCCTACATCGCCTTCAAGAGGTGGAACAGCTGCAAGCA
GAACAAGCAGGGCGCCAACAGCAGGCCCGTGAACCAGACCCCCCCCCCCGAGGGC
GAGAAGCTGCACAGCGACAGCGGCATCAGCGTGGACAGCCAGAGCCTGCACGACC
AGCAGCCCCACACCCAGACCGCCAGCGGCCAGGCCCTGAAGGGCGACGGCGGCCTG
TACAGCAGCCTGCCCCCCGCCAAGAGGGAGGAGGTGGAGAAGCTGCTGAACGGCA
GCGCCGGCGACACCTGGAGGCACCTGGCCGGCGAGCTGGGCTACCAGCCCGAGCAC
ATCGACAGCTTCACCCACGAGGCCTGCCCCGTGAGGGCCCTGCTGGCCAGCTGGGC
CACCCAGGACAGCGCCACCCTGGACGCCCTGCTGGCCGCCCTGAGGAGGATCCAGA
GGGCCGACCTGGTGGAGAGCCTGTGCAGCGAGAGCACCGCCACCAGCCCCGTG
Human p75NTR protein ECD with stalk region (SEQ ID NO: 73)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTPPE
GSDSTAPSTQEPEAPPEQDLIASTVAGVVTTVM
The WT ECD region includes the stalk region (underlined) and alpha and
gamma secretase cleavage 3′ of the stalk region (in bold)
Human p75NTR ECD nucleic acid sequence (SEQ ID NO: 74)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCGA
CGACGCCGTGTGCAGGTGCGCCTACGGCTACTACCAGGACGAGACCACCGGCAGGT
GCGAGGCCTGCAGGGTGTGCGAGGCCGGCAGCGGCCTGGTGTTCAGCTGCCAGGAC
AAGCAGAACACCGTGTGCGAGGAGTGCCCCGACGGCACCTACAGCGACGAGGCCA
ACCACGTGGACCCCTGCCTGCCCTGCACCGTGTGCGAGGACACCGAGAGGCAGCTG
AGGGAGTGCACCAGGTGGGCCGACGCCGAGTGCGAGGAGATCCCCGGCAGGTGGA
TCACCAGGAGCACCCCCCCCGAGGGCAGCGACAGCACCGCCCCCAGCACCCAGGAG
CCCGAGGCCCCCCCCGAGCAGGACCTGATCGCCAGCACCGTGGCCGGCGTGGTGAC
CACCGTGATG
Human ECD of p75NTR stalk region protein (SEQ ID NO: 75)
GRWITRSTPPEGSDSTAPSTQEPEAPPEQDLIASTVAGVVTTVM
The WT ECD stalk region (underlined) and alpha and gamma secretase
cleavage 3′ of the stalk region (in bold)
Human ECD of p75NTR stalk region nucleic acid sequence (SEQ ID NO: 76)
GGCAGGTGGATCACCAGGAGCACCCCCCCCGAGGGCAGCGACAGCACCGCCCCCAG
CACCCAGGAGCCCGAGGCCCCCCCCGAGCAGGACCTGATCGCCAGCACCGTGGCCG
GCGTGGTGACCACCGTGATG
Human IgG1 (SEQ ID NO: 77)
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
Human IgG2 (SEQ ID NO: 78)
ERKCCVECPPCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGLPAPIEKTI
SKTKGQPREPQVYTLPPSREEMTKNQVSLTCLVKGFYPSDISVEWESNGQPENNYKTTP
PMLDSDGSFFLYSKLTVDKSRWQQGNVFSCSVMHEALHNHYTQKSLSLSPGK
Human IgG3 (SEQ ID NO: 79)
ELKTPLGDTTHTCPRCPEPKSCDTPPPCPRCPEPKSCDTPPPCPRCPEPKSCDTPPPCPRCP
APELLGGPSVFLFPPKPKDTLMISRTPEVTCVVVDVSHEDPEVQFKWYVDGVEVHNAKT
KPREEQYNSTFRVVSVLTVLHQDWLNGKEYKCKVSNKALPAPIEKTISKTKGQPREPQV
YTLPPSREEMTKNQVSLTCLVKGFYPSDIAVEWESSGQPENNYNTTPPMLDSDGSFFLYS
KLTVDKSRWQQGNIFSCSVMHEALHNRFTQKSLSLSPGK
Human IgG4 (SEQ ID NO: 80)
ESKYGPPCPSCPAPEFLGGPSVFLFPPKPKDTLMISRTPEVTCVVVDVSQEDPEVQFNWY
VDGVEVHNAKTKPREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESNGQPENNYKTTPP
VLDSDGSFFLYSRLTVDKSRWQEGNVFSCSVMHEALHNHYTQKSLSLSLGK
PetML308_Human p75NTR ECD full stalk IgG1 (SEQ ID NO: 81)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTPPE
GSDSTAPSTQEPEAPPEQDLIASTVAGVVTTVMGGGGEPKSCDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDK
SRWQQGNVFSCSVMHEALHNHYTQKSLSLSPGK
PetML309_Human p75NTR ECD partial stalk IgG1 (SEQ ID NO: 82)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGRWITRSTPPE
GGGGEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K
PetML319_Human p75NTR ECD no stalk IgG1 (SEQ ID NO: 83)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVDPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human p75NTR ECD E75T (SEQ ID NO: 84)
KEACPTGLYTHSGECCKACNLGEGVAQPCGANQTVCEPCLDSVTFSDVVSATEPCKPCT
ECVGLQSMSAPCVEATDAVCRCAYGYYQDETTGRCEACRVCEAGSGLVFSCQDKQNT
VCEECPDGTYSDEANHVDPCLPCTVCEDTERQLRECTRWADAECEEIP
Human p75NTR ECD E75T-human IgG1 Fc (SEQ ID NO: 85)
KEACPTGLYTHSGECCKACNLGEGVAQPCGANQTVCEPCLDSVTFSDVVSATEPCKPCT
ECVGLQSMSAPCVEATDAVCRCAYGYYQDETTGRCEACRVCEAGSGLVFSCQDKQNT
VCEECPDGTYSDEANHVDPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human p75NTR ECD E75T-human IgG1 Fc nucleic acid sequence (SEQ ID NO: 86)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCAC
CGACGCCGTGTGCGGTGCGCCTACGGCTACTACCAGGACGAGACCACCGGCAGGTG
CGAGGCCTGCAGGGTGTGCGAGGCCGGCAGCGGCCTGGTGTTCAGCTGCCAGGACA
AGCAGAACACCGTGTGCGAGGAGTGCCCCGACGGCACCTACAGCGACGAGGCCAAC
CACGTGGACCCCTGCCTGCCCTGCACCGTGTGCGAGGACACCGAGAGGCAGCTGAG
GGAGTGCACCAGGTGGGCCGACGCCGAGTGCGAGGAGATCCCCGGCGGCGGCGGC
GAGCCCAAGAGCTGCGACAAGACCCACACCTGCCCCCCCTGCCCCGCCCCCGAGCT
GCTGGGCGGCCCCAGCGTGTTCCTGTTCCCCCCCAAGCCCAAGGACACCCTGATGAT
CAGCAGGACCCCCGAGGTGACCTGCGTGGTGGTGGACGTGAGCCACGAGGACCCCG
AGGTGAAGTTCAACTGGTACGTGGACGGCGTGGAGGTGCACAACGCCAAGACCAAG
CCCAGGGAGGAGCAGTACAACAGCACCTACAGGGTGGTGAGCGTGCTGACCGTGCT
GCACCAGGACTGGCTGAACGGCAAGGAGTACAAGTGCAAGGTGAGCAACAAGGCC
CTGCCCGCCCCCATCGAGAAGACCATCAGCAAGGCCAAGGGCCAGCCCAGGGAGCC
CCAGGTGTACACCCTGCCCCCCAGCAGGGACGAGCTGACCAAGAACCAGGTGAGCC
TGACCTGCCTGGTGAAGGGCTTCTACCCCAGCGACATCGCCGTGGAGTGGGAGAGC
AACGGCCAGCCCGAGAACAACTACAAGACCACCCCCCCCGTGCTGGACAGCGACGG
CAGCTTCTTCCTGTACAGCAAGCTGACCGTGGACAAGAGCAGGTGGCAGCAGGGCA
ACGTGTTCAGCTGCAGCGTGATGCACGAGGCCCTGCACAACCACTACACCCAGAAG
AGCCTGAGCCTGAGCCCCGGCAAG
Human p75NTR ECD S109Y (SEQ ID NO: 87)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFYCQDKQNT
VCEE CPDGTYSDEANHVDPCLPCTVCEDTERQLRECTRWADAECEEIP
Human p75NTR ECD S109Y-human IgG1 Fc (SEQ ID NO: 88)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFYCQDKQNT
VCEECPDGTYSDEANHVDPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human p75NTR ECD S109Y-human IgG1 Fc nucleic acid sequence (SEQ ID NO: 89)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCGA
CGACGCCGTGTGCAGGTGCGCCTACGGCTACTACCAGGACGAGACCACCGGCAGGT
GCGAGGCCTGCAGGGTGTGCGAGGCCGGCAGCGGCCTGGTGTTCTACTGCCAGGAC
AAGCAGAACACCGTGTGCGAGGAGTGCCCCGACGGCACCTACAGCGACGAGGCCA
ACCACGTGGACCCCTGCCTGCCCTGCACCGTGTGCGAGGACACCGAGAGGCAGCTG
AGGGAGTGCACCAGGTGGGCCGACGCCGAGTGCGAGGAGATCCCCGGCGGCGGCG
GCGAGCCCAAGAGCTGCGACAAGACCCACACCTGCCCCCCCTGCCCCGCCCCCGAG
CTGCTGGGCGGCCCCAGCGTGTTCCTGTTCCCCCCCAAGCCCAAGGACACCCTGATG
ATCAGCAGGACCCCCGAGGTGACCTGCGTGGTGGTGGACGTGAGCCACGAGGACCC
CGAGGTGAAGTTCAACTGGTACGTGGACGGCGTGGAGGTGCACAACGCCAAGACCA
AGCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCGCCCCCATCGAGAAGACCATCAGCAAGGCCAAGGGCCAGCCCAGGGAG
CCCCAGGTGTACACCCTGCCCCCCAGCAGGGACGAGCTGACCAAGAACCAGGTGAG
CCTGACCTGCCTGGTGAAGGGCTTCTACCCCAGCGACATCGCCGTGGAGTGGGAGA
GCAACGGCCAGCCCGAGAACAACTACAAGACCACCCCCCCCGTGCTGGACAGCGAC
GGCAGCTTCTTCCTGTACAGCAAGCTGACCGTGGACAAGAGCAGGTGGCAGCAGGG
CAACGTGTTCAGCTGCAGCGTGATGCACGAGGCCCTGCACAACCACTACACCCAGA
AGAGCCTGAGCCTGAGCCCCGGCAAG
Human p75NTR ECD S109H (SEQ ID NO: 90)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFHCQDKQNT
VCEECPDGTYSDEANHVDPCLPCTVCEDTERQLRECTRWADAECEEIP
Human p75NTR ECD S109H-human IgG1 Fc (SEQ ID NO: 91)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFHCQDKQNT
VCEECPDGTYSDEANHVDPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAEWESNGQPENNYKTTPPVL
DSDGSFFLYSKLTVDKSRWQQGNVFSCSVMHEALHNHYTQKSLSLSPGK
Human p75NTR ECD S109H-human IgG1 Fc nucleic acid sequence (SEQ ID NO: 92)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCGA
CGACGCCGTGTGCAGGTGCGCCTACGGCTACTACCAGGACGAGACCACCGGCAGGT
GCGAGGCCTGCAGGGTGTGCGAGGCCGGCAGCGGCCTGGTGTTCCACTGCCAGGAC
AAGCAGAACACCGTGTGCGAGGAGTGCCCCGACGGCACCTACAGCGACGAGGCCA
ACCACGTGGACCCCTGCCTGCCCTGCACCGTGTGCGAGGACACCGAGAGGCAGCTG
AGGGAGTGCACCAGGTGGGCCGACGCCGAGTGCGAGGAGATCCCCGGCGGCGGCG
GCGAGCCCAAGAGCTGCGACAAGACCCACACCTGCCCCCCCTGCCCCGCCCCCGAG
CTGCTGGGCGGCCCCAGCGTGTTCCTGTTCCCCCCCAAGCCCAAGGACACCCTGATG
ATCAGCAGGACCCCCGAGGTGACCTGCGTGGTGGTGGACGTGAGCCACGAGGACCC
CGAGGTGAAGTTCAACTGGTACGTGGACGGCGTGGAGGTGCACAACGCCAAGACCA
AGCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCGCCCCCATCGAGAAGACCATCAGCAAGGCCAAGGGCCAGCCCAGGGAG
CCCCAGGTGTACACCCTGCCCCCCAGCAGGGACGAGCTGACCAAGAACCAGGTGAG
CCTGACCTGCCTGGTGAAGGGCTTCTACCCCAGCGACATCGCCGTGGAGTGGGAGA
GCAACGGCCAGCCCGAGAACAACTACAAGACCACCCCCCCCGTGCTGGACAGCGAC
GGCAGCTTCTTCCTGTACAGCAAGCTGACCGTGGACAAGAGCAGGTGGCAGCAGGG
CAACGTGTTCAGCTGCAGCGTGATGCACGAGGCCCTGCACAACCACTACACCCAGA
AGAGCCTGAGCCTGAGCCCCGGCAAG
Human p75NTR ECD V133R (SEQ ID NO: 93)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHRDPCLPCTVCEDTERQLRECTRWADAECEEIP
Human p75NTR ECD V133R-human IgG1 Fc (SEQ ID NO: 94)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHRDPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human p75NTR ECD V133R-human IgG1 Fc nucleic acid sequence (SEQ ID NO: 95)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCGA
CGACGCCGTGTGCAGGTGCGCCTACGGCTACTACCAGGACGAGACCACCGGCAGGT
GCGAGGCCTGCAGGGTGTGCGAGGCCGGCAGCGGCCTGGTGTTCAGCTGCCAGGAC
AAGCAGAACACCGTGTGCGAGGAGTGCCCCGACGGCACCTACAGCGACGAGGCCA
ACCACAGGGACCCCTGCCTGCCCTGCACCGTGTGCGAGGACACCGAGAGGCAGCTG
AGGGAGTGCACCAGGTGGGCCGACGCCGAGTGCGAGGAGATCCCCGGCGGCGGCG
GCGAGCCCAAGAGCTGCGACAAGACCCACACCTGCCCCCCCTGCCCCGCCCCCGAG
CTGCTGGGCGGCCCCAGCGTGTTCCTGTTCCCCCCCAAGCCCAAGGACACCCTGATG
ATCAGCAGGACCCCCGAGGTGACCTGCGTGGTGGTGGACGTGAGCCACGAGGACCC
CGAGGTGAAGTTCAACTGGTACGTGGACGGCGTGGAGGTGCACAACGCCAAGACCA
AGCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCGCCCCCATCGAGAAGACCATCAGCAAGGCCAAGGGCCAGCCCAGGGAG
CCCCAGGTGTACACCCTGCCCCCCAGCAGGGACGAGCTGACCAAGAACCAGGTGAG
CCTGACCTGCCTGGTGAAGGGCTTCTACCCCAGCGACATCGCCGTGGAGTGGGAGA
GCAACGGCCAGCCCGAGAACAACTACAAGACCACCCCCCCCGTGCTGGACAGCGAC
GGCAGCTTCTTCCTGTACAGCAAGCTGACCGTGGACAAGAGCAGGTGGCAGCAGGG
CAACGTGTTCAGCTGCAGCGTGATGCACGAGGCCCTGCACAACCACTACACCCAGA
AGAGCCTGAGCCTGAGCCCCGGCAAG
Human p75NTR ECD D134L (SEQ ID NO: 96)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVLPCLPCTVCEDTERQLRECTRWADAECEEIP
Human p75NTR ECD D134L-human IgG1 Fc (SEQ ID NO: 97)
KEACPTGLYTHSGECCKACNLGEGVAQPCGANQTVCEPCLDSVTFSDVVSATEPCKPCT
ECVGLQSMSAPCVEADDAVCRCAYGYYQDETTGRCEACRVCEAGSGLVFSCQDKQNT
VCEECPDGTYSDEANHVLPCLPCTVCEDTERQLRECTRWADAECEEIPGGGGEPKSCDK
THTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
Human p75NTR ECD D134L-human IgG1 Fc nucleic acid sequence (SEQ ID NO: 98)
AAGGAGGCCTGCCCCACCGGCCTGTACACCCACAGCGGCGAGTGCTGCAAGGCCTG
CAACCTGGGCGAGGGCGTGGCCCAGCCCTGCGGCGCCAACCAGACCGTGTGCGAGC
CCTGCCTGGACAGCGTGACCTTCAGCGACGTGGTGAGCGCCACCGAGCCCTGCAAG
CCCTGCACCGAGTGCGTGGGCCTGCAGAGCATGAGCGCCCCCTGCGTGGAGGCCGA
CGACGCCGTGTGCAGGTGCGCCTACGGCTACTACCAGGACGAGACCACCGGCAGGT
GCGAGGCCTGCAGGGTGTGCGAGGCCGGCAGCGGCCTGGTGTTCAGCTGCCAGGAC
AAGCAGAACACCGTGTGCGAGGAGTGCCCCGACGGCACCTACAGCGACGAGGCCA
ACCACGTGCTGCCCTGCCTGCCCTGCACCGTGTGCGAGGACACCGAGAGGCAGCTG
AGGGAGTGCACCAGGTGGGCCGACGCCGAGTGCGAGGAGATCCCCGGCGGCGGCG
GCGAGCCCAAGAGCTGCGACAAGACCCACACCTGCCCCCCCTGCCCCGCCCCCGAG
CTGCTGGGCGGCCCCAGCGTGTTCCTGTTCCCCCCCAAGCCCAAGGACACCCTGATG
ATCAGCAGGACCCCCGAGGTGACCTGCGTGGTGGTGGACGTGAGCCACGAGGACCC
CGAGGTGAAGTTCAACTGGTACGTGGACGGCGTGGAGGTGCACAACGCCAAGACCA
AGCCCAGGGAGGAGCAGTACAACAGCACCTACAGGGTGGTGAGCGTGCTGACCGTG
CTGCACCAGGACTGGCTGAACGGCAAGGAGTACAAGTGCAAGGTGAGCAACAAGG
CCCTGCCCGCCCCCATCGAGAAGACCATCAGCAAGGCCAAGGGCCAGCCCAGGGAG
CCCCAGGTGTACACCCTGCCCCCCAGCAGGGACGAGCTGACCAAGAACCAGGTGAG
CCTGACCTGCCTGGTGAAGGGCTTCTACCCCAGCGACATCGCCGTGGAGTGGGAGA
GCAACGGCCAGCCCGAGAACAACTACAAGACCACCCCCCCCGTGCTGGACAGCGAC
GGCAGCTTCTTCCTGTACAGCAAGCTGACCGTGGACAAGAGCAGGTGGCAGCAGGG
CAACGTGTTCAGCTGCAGCGTGATGCACGAGGCCCTGCACAACCACTACACCCAGA
AGAGCCTGAGCCTGAGCCCCGGCAAG

Examples of p75NTR proteins of the invention from camels and pigs are incorporated in the Sequence Listing XML as follows:
Camel p75NTR protein (SEQ ID NO:99)
Camel p75NTR nucleic acid sequence (SEQ ID NO:100)
Camel p75NTR protein ECD (SEQ ID NO:101)
Camel p75NTR protein ECD nucleic acid sequence (SEQ ID NO:102)
Camel p75NTR ECD E75T (SEQ ID NO:103)
Camel p75NTR ECD S109Y (SEQ ID NO:104)
Camel p75NTR ECD S109H (SEQ ID NO:105)
Camel p75NTR ECD V133R (SEQ ID NO:106)
Camel p75NTR ECD D134L (SEQ ID NO:107)
Pig p75NTR protein (SEQ ID NO:108)
Pig p75NTR nucleic acid sequence (SEQ ID NO:109)
Pig p75NTR protein ECD (SEQ ID NO:110)
Pig p75NTR protein ECD nucleic acid sequence (SEQ ID NO:111)
Pig p75NTR ECD E75T (SEQ ID NO:112)
Pig p75NTR ECD S109Y (SEQ ID NO:113)
Pig p75NTR ECD S109H (SEQ ID NO:114)
Pig p75NTR ECD V133R (SEQ ID NO:115)
Pig p75NTR ECD D134L (SEQ ID NO:116)
Equine p75NTR protein ECD (SEQ ID NO:118)
Equine p75NTR protein ECD nucleic acid sequence (SEQ ID NO:119)
Equine p75NTR ECD E75T (SEQ ID NO:120)
Equine p75NTR ECD S109Y (SEQ ID NO:121)
Equine p75NTR ECD S109H (SEQ ID NO:122)
Equine p75NTR ECD V133R (SEQ ID NO:123)
Equine p75NTR ECD D134L (SEQ ID NO:124)
Bovine p75NTR protein ECD (SEQ ID NO:125)
Bovine p75NTR protein ECD nucleic acid sequence (SEQ ID NO:126)
Bovine p75NTR ECD E75T (SEQ ID NO:127)
Bovine p75NTR ECD S109Y (SEQ ID NO:128)
Bovine p75NTR ECD S109H (SEQ ID NO:129)
Bovine p75NTR ECD V133R (SEQ ID NO:130)
Bovine p75NTR ECD D134L (SEQ ID NO:131)

1. An isolated polypeptide comprising a companion animal p75 neurotrophin receptor (p75NTR) extracellular domain, wherein said p75NTR comprises a variant amino acid at one or more of positions 109 and 133.

2. The isolated polypeptide of claim 1, wherein the variant amino acid at position 109 of p75NTR comprises an aromatic side chain, histidine, tyrosine, phenylalanine, or tryptophan and/or the variant amino acid at position 133 of p75NTR comprises a charged side chain, arginine, histidine, or lysine.

3. The isolated polypeptide of claim 1, wherein the companion animal is a cat, dog, pig, cow, horse, or camel.

4. The isolated polypeptide of claim 1,
wherein the companion animal is a dog and the p75NTR comprises or consists of a sequence at least 95% identical to SEQ ID NO:49, SEQ ID NO:52, or SEQ ID NO:55, or
wherein the companion animal is a cat and the p75NTR comprises or consists of a sequence at least 95% identical to SEQ ID NO:63, SEQ ID NO:65, or SEQ ID NO:67, or
wherein the companion animal is a horse and the p75NTR comprises or consists of a sequence at least 95% identical to SEQ ID NO:121, SEQ ID NO:122, or SEQ ID NO:123, or
wherein the companion animal is a cow and the p75NTR comprises or consists of a sequence at least 95% identical to SEQ ID NO:128, SEQ ID NO:129, or SEQ ID NO:130, or
wherein the companion animal is a pig and the p75NTR comprises or consists of a sequence at least 95% identical to SEQ ID NO:113, SEQ ID NO:114, or SEQ ID NO:115, or
wherein the companion animal is a camel and the p75NTR comprises or consists of a sequence at least 95% identical to SEQ ID NO:104, SEQ ID NO:105, SEQ ID NO:106.

5. The isolated polypeptide of claim 1, wherein the p75NTR extracellular domain is truncated.

6. An isolated nucleic acid encoding the isolated polypeptide of claim 1.

7. A vector comprising the nucleic acid of claim 6.

8. A host cell comprising the vector of claim 7.

9. A fusion protein comprising a companion animal p75NTR extracellular domain, wherein said p75NTR comprises a variant amino acid at one or more of position 109 and 133 and a half-life extending moiety.

10. The fusion protein of claim 9, wherein the half-life extending moiety comprises a wild type Fc domain, a mutant Fc domain, a serum albumin binder or PEG.

11. The fusion protein of claim 9, wherein the half-life extending moiety comprises an Fc domain, and the p75NTR extracellular domain or portion thereof and the Fc domain are linked with a linker.

12. The fusion protein of claim 11, wherein the linker comprises (GGGG)ₙ or (G₄S)ₙ wherein n is 1 to 4.

13. The fusion protein of claim 9, wherein the companion animal is a cat, dog, pig, cow, horse or camel.

14. The fusion protein of claim 9, wherein the companion animal is a dog and the Fc domain comprises a canine Fc domain.

15. The fusion protein of claim 14, wherein the fusion protein comprises or consists of a sequence at least 95% identical to SEQ ID NO:50, SEQ ID NO:53, or SEQ ID NO:56.

16. The fusion protein of claim 9, wherein the Fc domain comprises a feline Fc domain.

17. The fusion protein of claim 16, wherein the fusion protein comprises or consists of a sequence at least 95% identical to SEQ ID NO:64, SEQ ID NO:66, or SEQ ID NO:68.

18. A nucleic acid encoding the fusion protein of claim 9.

19. A vector comprising the nucleic acid of claim 18.

20. A host cell comprising the vector of claim 19.

21. A pharmaceutical composition comprising the isolated polypeptide of claim 1.

22. A pharmaceutical composition comprising the fusion protein of claim 9.

23. A method for treating an NGF-related disorder in a companion animal comprising administering to the companion animal an effective amount of the p75NTR polypeptide of claim 1.

24. A method for treating an NGF-related disorder in a companion animal comprising administering to the companion animal an effective amount of the fusion protein of claim 9.

25. The method of claim 24, wherein the NGF-related disorder comprises cardiovascular diseases, atherosclerosis, obesity, type 2 diabetes, metabolic syndrome, inflammation, pain, osteoarthritis pain, rheumatoid arthritis pain, surgical and postsurgical pain, incisional pain, general inflammatory pain, cancer pain, pain from trauma, neuropathic pain, neuralgia, diabetic neuropathy pain, pain associated with rheumatic diseases, pain associated with musculoskeletal diseases, visceral pain, or gastrointestinal pain.

26. A method of inhibiting NGF activity in a companion animal, which comprises administering to the companion animal a p75NTR polypeptide of claim 1.

27. A method of inhibiting NGF activity in a companion animal, which comprises administering to the companion animal the fusion protein of claim 9.

28. The method of claim 23, wherein the fusion protein is administered with a second agent that treats pain.

29. A kit comprising the isolated companion animal p75NTR polypeptide of claim 1, and instructions for use.

30. A kit comprising the fusion protein of claim 9, and instructions for use.


SEQUENCE LISTING (131 sequences)

SEQ ID NO: 1 - protein, 399 aa, Canis lupus
  source 1..399: note = subspecies familiaris
  DOMAIN 1..222: note = Extracellular Domain
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEDSDSTAPSTEEPELPPDQEIIASTMADVVTTVMGSSQPVVTRGTADNLIPVYCSILAAVVVGLVAYIAFKRWNSCKQNKQGANSRPVNQTPPPEGEKLHSDSGISVDSQSLHDQQPHTQTAAGQALKGDGGLYSSLPPAKREEVEKLLNGSAGDTWRHLAGELGYQPEHIDSFTHEACPARALLASWAAQDSATLDALLAALRRIQRADIVESLCSESTATSPV

SEQ ID NO: 2 - DNA, 1263 bp, Canis lupus
  source 1..1263: mol_type = other DNA; sub_species = familiaris
  sig_peptide 1..63
atggacgggccgcgcctgctgctgctgctgctgctgctcctgggggtgtcccttggaggtgccaaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgttggattacccggtccacaccctcagaggactcggacagcaccgcccccagcacagaggagccagagctacctccagatcaagaaatcatagccagcaccatggcagatgtggtgaccacagtgatgggcagctctcagcctgtagtgacccgaggaaccgctgacaacctcatccctgtctactgctccatcctggccgccgtggttgtgggcttagtggcctacattgccttcaagaggtggaacagctgcaagcagaacaagcaaggagccaacagccggcccgtgaaccagacgcctccgccggagggagaaaagctccacagtgacagtggcatctctgtggacagccagagcctgcatgaccagcagccccacacacagacggccgcaggccaggccctcaagggggatggaggtctctacagcagcctgccaccagccaagcgggaggaggtggagaagctgctcaatggctctgcgggggacacctggcggcacctggcaggtgagctgggctaccagcctgagcacatagactccttcacccacgaggcctgcccagcccgagccctgcttgccagctgggccgcccaggacagcgcgacgctcgacgccctcctggctgctctgcgccgcatccagcgagccgacatcgtggagagcctgtgtagcgagtccacggccacgtctccagtgtga

SEQ ID NO: 3 - protein, 399 aa, Felis catus
  DOMAIN 1..222: note = Extracellular Domain
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEGSDSTAPSTEEPEVPPEQDLIASTVADVVTTVMGSSQPVVTRGTADNLIPVYCSILAAVVVGLVAYIAFKRWNSCKQDKQGANSRPVNQTPPPEGEKLHSDSGISVDSQSLHDQQSHTQTAAGQALKGDGGLYSSLPSAKREEVEKLLNGSAGDTWRHLAGELGYQPEHIDSFTREACPARALLASWAAQDSATLDALLAALRRIQRADIVESLCSESTATSPV

SEQ ID NO: 4 - DNA, 1266 bp, Felis catus
  source 1..1266: mol_type = other DNA
  sig_peptide 1..66
atggacgggccgcgcccgctgctgctgctgttgccgctgctcctgggggtgtcccttggaggtgccaaggaggcatgtcccacgggcctgttcacccacagcggcgagtgctgtaaagcctgcaacctgggagagggcgtagcccagccttgcggagccaaccagaccgtgtgtgagccctgcctggacagcgtgaccttctcggacgtggtgagcgccacggagccgtgcaagccgtgcaccgagtgcgtgggcctgcagagcatgtcggcgccgtgcgtggaggccgacgacgccgtgtgtcgctgcgcctacggctactaccaggacgagacgacgggccgctgcgaggcgtgccgcgtgtgcgaggcgggctccggcctggtgttctcgtgccaggaccggcagaataccgtgtgcgaggagtgtcccgacggcacgtactcggacgaggccaaccacgtggacccgtgcctgccctgcaccgtgtgcgaggacaccgagcgccagctgcgcgagtgcacgcgctgggccgacgccgagtgcgaggagatccctggccgttggattactcggtctacaccttcggagggctcggacagcaccgcccccagcacggaggagccagaggtacctccagagcaagacctcatagccagcacggtggcagatgtggtgaccacagtgatgggcagctctcagcccgtagtgacccgaggcaccgccgacaacctcatccctgtctattgttccatcctggccgctgtggttgtgggcctggtggcctacattgccttcaagaggtggaacagctgcaaacaggacaagcaaggcgccaacagccggcccgtgaaccagacgcccccgcccgagggagaaaagctccacagtgacagtggcatctctgtggacagccagagcctgcatgaccagcagtcccacacgcagacggccgccggccaggccctcaagggggacggaggtctctacagcagcctgccgtcagccaagcgggaggaggtagagaaactgctcaacggctctgcgggggacacgtggcggcacctggcgggcgagctgggctaccagcctgagcacatagactccttcacccgcgaggcctgcccagcccgggccctgctcgccagctgggccgcccaggacagcgcgacgctcgacgccctcctggccgccctgcgccgcatccagcgggccgacatcgtcgagagcctgtgcagcgagtccacggccacgtccccggtgtga

SEQ ID NO: 5 - protein, 405 aa, Equus caballus
  DOMAIN 1..222: note = Extracellular Domain
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFSCQDKQNTVCEECPDGTYSDEANHVDPCLPCTVCEDTERQLRECTRWADAECEEIPSRWITRATPPEGSDSTAPSTQEPEGPPEKDLVASTVADVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTAAGQALKGDGGLYSSLPLAKREEVEKLLNGSAGDTWRHLAGLVGQGLLRLELVSVFQGPAHGGMLPPATPSLQAPVWLGPEGCSEKWDQRGNAARRAGLRVWPMEGLSQV

SEQ ID NO: 6 - DNA, 1290 bp, Equus caballus
  source 1..1290: mol_type = other DNA
  sig_peptide 1..90
atgagggcaggtgccgccgactgcgccatggacggaccgcgccttctgctgctgcttctgctcttgggggtgtgcctgctgggaggtgccaaggaggtgtgccccacagacctgtacacccacagcggcgagtgctgcaaagcctgcaacctgggcgagggtgtggcccagccttgcggagccaaccagactgtgtgtgaaccctgcctggacagcgtgacgttctcggacgtggtgagcgccacagagccatgtaagccgtgcaccgagtgcgtgggcctgcagagcatgtcggcgccatgcgtggaggccgacgacgcggtgtgccgctgcgcctatggctactaccaggacgagacgacgggccgctgcgaggcgtgccaggtgtgcgaggcgggctcgggcctcgtgttctcgtgccaggacaagcagaacaccgtgtgcgaggaatgccccgacggcacgtactccgacgaggccaaccacgtggacccgtgcctgccctgcaccgtgtgcgaggacaccgagcgccagctgcgagagtgcacgcgctgggccgacgccgagtgcgaggagatccccagccgttggattacacgggccacgccgccggagggctcagacagcactgcccccagcacccaggagcccgagggacctccagagaaagaccttgtagccagcacggtggcggatgtggtgaccacagtgatgggcagctctcagcccgtggtgacccgaggcaccacggacaacctcatccccgtctattgctccatcctggccgctgtggttgtgggccttgtggcctacatcgccttcaagaggtggaacagctgcaagcagaacaagcaaggagccaacagccgacccgtgaaccagacaccaccacccgagggagaaaaactccacagcgacagcggcatctctgtggacagccagagcctgcatgaccagcagcctcacacacagacagccgcaggccaggccctcaagggagatggaggcctctacagcagcctgccactggccaagagggaagaggtggagaagctactcaatggctccgcaggggacacctggcggcacctggcgggtgagctgggctaccagcccgagcacatagactccttcacccacgaggcctgccccgtccgcgccctgcttgccagctgggccgcccaggacagtgcgacattcgatgccctcctgaccgccctgcgccgcatccagcgagccgacattgtcgagagcctgtgcagcgagtccaccgccacatccccggtgtga

SEQ ID NO: 7 - protein, 222 aa, Canis lupus
  source 1..222: note = subspecies familiaris
  REGION 167..208: note = stalk region
  REGION 208..222: note = alpha and gamma secretase cleavage
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEDSDSTAPSTEEPELPPDQEIIASTMADVVTTVMGSSQPVVTRGTADN

SEQ ID NO: 8 - DNA, 666 bp, Canis lupus
  source 1..666: mol_type = other DNA; sub_species = familiaris
aaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgttggattacccggtccacaccctcagaggactcggacagcaccgcccccagcacagaggagccagagctacctccagatcaagaaatcatagccagcaccatggcagatgtggtgaccacagtgatgggcagctctcagcctgtagtgacccgaggaaccgctgacaac

SEQ ID NO: 9 - protein, 42 aa, Canis lupus
  source 1..42: note = subspecies familiaris
WITRSTPSEDSDSTAPSTEEPELPPDQEIIASTMADVVTTVM

SEQ ID NO: 10 - DNA, 126 bp, Canis lupus
  source 1..126: mol_type = other DNA; sub_species = familiaris
tggattacccggtccacaccctcagaggactcggacagcaccgcccccagcacagaggagccagagctacctccagatcaagaaatcatagccagcaccatggcagatgtggtgaccacagtgatg

SEQ ID NO: 11 - protein, 422 aa, Canis lupus
  source 1..422: note = subspecies familiaris
  SIGNAL 1..19
  REGION 188..422: note = canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 12 - DNA, 1269 bp, Canis lupus
  source 1..1269: mol_type = other DNA; sub_species = familiaris
  sig_peptide 1..57
atggaatggtcctgggtgttcctgttcttcctgtccgtgaccaccggcgtgcactccaaagaggcttgtcctaccggcctgtacacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaggtggcggacgcgagaatggcagagtgcctagacctcctgactgccctaagtgccctgctcctgaaatgctcggcggaccctccgtgttcatcttcccacctaagcctaaggacaccctgctgatcgctcggacccctgaagtgacatgcgtggtggtggatctggaccccgaggatcctgaggtgcagatcagttggttcgtggacggcaagcagatgcagaccgctaagacccagcctagagaggaacagttcaacggcacctacagagtggtgtctgtgctgcctatcggccaccaggattggctgaagggcaagcagtttacctgcaaagtgaacaacaaggccctgccttctccaatcgagcggaccatctctaaggccagaggccaggctcatcagccttccgtgtatgtcctgccacctagccgcgaggaactgtccaagaacaccgtgtctctgacctgcctgatcaaggacttcttccctcctgacatcgacgtggaatggcagtccaacggccagcaagagcccgagtctaagtaccggacaacccctccacagctggacgaggacggctcctacttcctgtactccaagctgtccgtggacaagtctcggtggcagagaggcgacaccttcatctgtgctgtgatgcacgaggccctgcacaaccactacacccagaagtccctgtctcactcccctggcaagtga

SEQ ID NO: 13 - protein, 422 aa, Canis lupus
  source 1..422: note = subspecies familiaris
  SIGNAL 1..19
  REGION 188..422: note = canine Fc-B-YTE
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGRENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 14 - DNA, 1269 bp, Canis lupus
  source 1..1269: mol_type = other DNA; sub_species = familiaris
  sig_peptide 1..57
atggaatggtcctgggtgttcctgttcttcctgtccgtgaccaccggcgtgcactccaaagaggcttgtcctaccggcctgtacacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaggtggcggacgcgagaatggcagagtgcctagacctcctgactgccctaagtgccctgctcctgaaatgctcggcggaccctccgtgttcatcttcccacctaagcctaaggacaccctgtatatcactcgggaacctgaagtgacatgcgtggtggtggatctggaccccgaggatcctgaggtgcagatcagttggttcgtggacggcaagcagatgcagaccgctaagacccagcctagagaggaacagttcaacggcacctacagagtggtgtctgtgctgcctatcggccaccaggattggctgaagggcaagcagtttacctgcaaagtgaacaacaaggccctgccttctccaatcgagcggaccatctctaaggccagaggccaggctcatcagccttccgtgtatgtcctgccacctagccgcgaggaactgtccaagaacaccgtgtctctgacctgcctgatcaaggacttcttccctcctgacatcgacgtggaatggcagtccaacggccagcaagagcccgagtctaagtaccggacaacccctccacagctggacgaggacggctcctacttcctgtactccaagctgtccgtggacaagtctcggtggcagagaggcgacaccttcatctgtgctgtgatgcacgaggccctgcacaaccactacacccagaagtccctgtctcactcccctggcaagtga

SEQ ID NO: 15 - protein, 471 aa, Canis lupus
  source 1..471: note = subspecies familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVFNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 16 - protein, 475 aa, Canis lupus
  source 1..475: note = subspecies familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 17 - protein, 473 aa, Canis lupus
  source 1..473: note = subspecies familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASTTAPSVFPLAPSCGSQSGSTVALACLVSGYIPEPVTVSWNSGSLTSGVHTFPSILQSSGLYSLSSMVTVPSSRWPSETFTCNVAHPATNTKVDKPVVKECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 18 - protein, 471 aa, Canis lupus
  source 1..471: note = subspecies familiaris
MEFVLGWVFLVAILQGVQGEVQLVESGGDLVKPAGSLRLSCVASGFTFSNNAMNWVRQAPGKGLQWVAGINSGGSTASADAVKGRFTISRDNAKNTVYLQMNSLTAEDTAVYYCAKVIGNWIATSDLDYWGQGTLVIVSSASSTAPSVFPLAPSCGSTSGSTVALACLVSGYFPEPVTVSWNSGSLTSGVHTFPSVLKSSGLYSLSSMVTVPSSRLPSETFTCNVVHPATNTKVDKPVPKESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 19 - protein, 233 aa, Canis lupus
  source 1..233: note = subspecies familiaris
FNECRCTDTPPCPVPEPLGGPSVLIFPPKPKDILRITRTPEVTCVVLDLGREDPEVQISWFVDGKEVHTAKTQSREQQFNGTYRVVSVLPIEHQDWLTGKEFKCRVNHIDLPSPIERTISKARGRAHKPSVYVLPPSPKELSSSDTVSITCLIKDFYPPDIDVEWQSNGQQEPERKHRMTPPQLDEDGSYFLYSKLSVDKSRWQQGDPFTCAVMHETLQNHYTDLSLSHSPGK

SEQ ID NO: 20 - protein, 235 aa, Canis lupus
  source 1..235: note = subspecies familiaris
RENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 21 - protein, 235 aa, Canis lupus
  source 1..235: note = subspecies familiaris
RENGRVPRPPDCPKCPAPEMLGGPSVFIFPPKPKDTLYITREPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 22 - protein, 233 aa, Canis lupus
  source 1..233: note = subspecies familiaris
ECECKCNCNNCPCPGCGLLGGPSVFIFPPKPKDILVTARTPTVTCVVVDLDPENPEVQISWFVDSKQVQTANTQPREEQSNGTYRVVSVLPIGHQDWLSGKQFKCKVNNKALPSPIEEIISKTPGQAHQPNVYVLPPSRDEMSKNTVTLTCLVKDFFPPEIDVEWQSNGQQEPESKYRMTPPQLDEDGSYFLYSKLSVDKSRWQRGDTFICAVMHEALHNHYTQKSLSHSPGK

SEQ ID NO: 23 - protein, 231 aa, Canis lupus
  source 1..231: note = subspecies familiaris
ESTCKCISPCPVPESLGGPSVFIFPPKPKDILRITRTPEVTCVVLDLGREDPEVQISWFVDGKEVHTAKTQPREQQFNSTYRVVSVLPIEHQDWLTGKEFKCRVNHIGLPSPIERTISKARGQAHQPGVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 24 - protein, 235 aa, Felis catus
TDHPPGPKPCDCPKCPPPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 25 - protein, 235 aa, Felis catus; identical to SEQ ID NO: 24

SEQ ID NO: 26 - protein, 236 aa, Felis catus
KTASTIESKTGEGPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVNSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 27 - protein, 229 aa, Equus caballus
VIKECNGGCPAECLQVGPSVFIFPPKPKDVLMISRTPTVTCVVVDVGHDFPDVQFNWYVDGVETHTATTEPKQEQFNSTYRVVSVLPIQHKDWLSGKEFKCKVNNKALPAPVERTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 28 - protein, 242 aa, Equus caballus
CVLSAEGVIPIPSVPKPQCPPYTHSKFLGGPSVFIFPPNPKDALMISRTPVVTCVVVNLSDQYPDVQFSWYVDNTEVHSAITKQREAQFNSTYRVVSVLPIQHQDWLSGKEFKCSVTNVGVPQPISRAISRGKGPSRVPQVYVLPPHPDELAKSKVSVTCLVKDFYPPDISVEWQSNRWPELEGKYSTTPAQLDGDGSYFLYSKLSLETSRWQQVESFTCAVMHEALHNHFTKTDISESLGK

SEQ ID NO: 29 - protein, 232 aa, Equus caballus
TTPPCPCECPKCPAPELLGGPSVFIFPPKPKDVLMITRTPEVTCLVVDVSHDSSDVLFTWYVDGTEVKTAKTMPNEEQNNSTYRVVSVLRIQHQDWLNGKKFKCKVNNQALPAPVERTISKATGQTRVPQVYVLAPHPDELSKNKVSVTCLVKDFLPTDITVEWQSNEHPEPEGKYRTTEAQKDSDGSYFLYSKLTVETDRWQQGTTFTCVVMHEALHNHVMQKNVSHSPGK

SEQ ID NO: 30 - protein, 229 aa, Equus caballus; identical to SEQ ID NO: 27

SEQ ID NO: 31 - protein, 230 aa, Equus caballus
VVKGSPCPKCPAPELPGGPSVFIFPPKPKDVLKISRKPEVTCVVVDLGHDDPDVQFTWFVDGVETHTATTEPKEEQFNSTYRVVSVLPIQHQDWLSGKEFKCSVTNKALPAPVERTTSKAKGQLRVPQVYVLAPHPDELAKNTVSVTCLVKDFYPPEIDVEWQSNEHPEPEGKYSTTPAQLNSDGSYFLYSKLSVETSRWKQGESFTCGVMHEAVENHYTQKNVSHSPGK

SEQ ID NO: 32 - protein, 224 aa, Equus caballus
KEPCCCPKCPGRPSVFIFPPNPKDTLMISRTPEVTCVVVDVSQENPDVKFNWYVDGVEAHTATTKAKEKQDNSTYRVVSVLPIQHQDWRRGKEFKCKVNNRALPAPVERTITKAKGELQDPKVYILAPHREEVTKNTVSVTCLVKDFYPPDINVEWQSNEEPEPEVKYSTTPAQLDGDGSYFLYSKLTVETDRWEQGESFTCVVMHEAIRHTYRQKSITNFPGK

SEQ ID NO: 33 - protein, 230 aa, Equus caballus
VIKECGGCPTCPECLSVGPSVFIFPPKPKDVLMISRTPTVTCVVVDVGHDFPDVQFNWYVDGVETHTATTEPKQEQNNSTYRVVSILAIQHKDWLSGKEFKCKVNNQALPAPVQKTISKPTGQPREPQVYVLAPHRDELSKNKVSVTCLVKDFYPTDIDIEWKSNGQPEPETKYSTTPAQLDSDGSYFLYSKLTVETNRWQQGTTFTCAVMHEALHNHYTEKSVSKSPGK

SEQ ID NO: 34 - protein, 164 aa, Canis lupus
  source 1..164: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIP

SEQ ID NO: 35 - DNA, 498 bp, Canis lupus
  source 1..498: mol_type = other DNA; sub_species = familiaris
aaggaggcatgtcccactggcctgtacacccacagcggcgagtgctgcaaagcctgcaatctgggtgagggggtggcccagccttgcggagccaaccagaccgtgtgtgagccctgcctggacagcgtgaccttctcggacgtggtgagcgccaccgagccgtgcaagccgtgcaccgagtgcgtggggctgcagagcatgtcggcgccgtgcgtggaggcggacgacgccgtgtgccgctgcgcctacggctactaccaggacgagacgacgggccgctgcgaggcgtgccgcgtgtgcgaggcgggctcggggctcgtgttctcgtgccaggacaggcagaacaccgtgtgcgaggagtgtcccgacggcacgtactccgacgaggccaaccacgtggacccgtgcctgccctgcaccgtgtgcgaggacaccgagcgccagctgcgcgagtgcacgcgctgggccgacgccgagtgcgaggagatccctggccgt

SEQ ID NO: 36 - protein, 399 aa, Bos taurus
  DOMAIN 1..222: note = Extracellular domain
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRATPPEGSDSTDPSTQEPEVPPEQDLVTSTVSDVVTTVMGSSQPVVTRGTADNLIPVYCSILAAVVVGLVAYIAFKRWNSCKQNKQGANSRPVNQTPPPEGEKLHSDSGISVDSQSLHDQQPHTQTAAGQALKGDGGLYSSLPLAKREEVEKLLNGSAGDTWRHLAGELGYQPEHIDSFTHEACPARALLASWAAQDSATLDTLLAALRRIQRADLVESLCSESTATSPV

SEQ ID NO: 37 - DNA, 1290 bp, Bos taurus
  source 1..1290: mol_type = other DNA
atggggtcaggtgccgccggccgcgccatggacgggccgcgcctgctgctgctgctgctgctgctcctgggggtgtcccttggaggtgccaaggaagcatgcctcacgggcctgtacacccacagcggagagtgctgcaaagcctgcaacctgggcgagggtgtggcccagccttgtggagccaaccagaccgtgtgtgaaccctgcctggacagcgtgaccttctcggacgtggtgagcgccacggagccgtgtaagccgtgcacggagtgcgtgggactgcagagcatgtcggcgccctgcgtggaggccgacgacgccgtgtgccgctgcgcctacggctattaccaggacgagacgaccggccgctgcgaggcgtgccgcgtgtgcgaggcgggctcggggctcgtgttctcgtgccaggacaagcagaacaccgtctgcgaggagtgccccgacggcacgtactccgacgaggccaaccacgtggacccctgcctgccctgcacggtgtgcgaggacacggagcgccagctgcgcgagtgcacgcgctgggccgacgccgagtgcgaggagatccctggacgttggattacacgggccacgccccctgagggctccgacagcacagaccccagcacccaggagcccgaggtacctccagagcaagatctggtaaccagcactgtgtcagatgtggtgaccacggtgatgggcagctcccagcctgtggtgacccgaggtaccgccgacaacctcatccctgtctattgctccatcctggctgctgtggttgtgggccttgtggcctacatcgccttcaagaggtggaacagctgcaagcagaacaagcaaggagccaacagccgacctgtgaaccagacacccccaccagagggggaaaagctacacagcgatagcggcatctctgtggacagccagagcctgcatgaccagcagccccacacgcagactgccgcaggccaggccctcaagggtgatggaggcctctacagcagcctgccgctggccaagcgggaggaggtggagaagctgctcaacggctctgcgggggacacctggcggcatctggcaggcgagttgggttaccagcctgagcacatagactccttcacccacgaggcctgcccagcccgcgccctgctggccagctgggctgcccaggacagcgccacgctcgacaccctccttgcggccctgcgccgcatccagcgcgccgacatcgtggagagcctgtgcagcgagtccacggccacgtcccccgtgtga

SEQ ID NO: 38 - protein, 222 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRWITRSTPSEGSDSTAPSTEEPEVPPEQDLIASTVADVVTTVMGSSQPVVTRGTADN

SEQ ID NO: 39 - protein, 429 aa, Felis catus
  SIGNAL 1..22
  REGION 193..428: note = Feline Fc-IgG2
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGRGGGGVPKTASTIESKTCDCPKCPVPEIPGAPSVFIFPPKPKDTLSISRTPEVTCLVVDLGPDDSNVQITWFVDNTEMHTAKTRPREEQFNSTYRVVSVLPILHQDWLKGKEFKCKVSKSLPSAMERTISKAKGQPHEPQVYVLPPTQEELSENKVSVTCLIKGFHPPDIAVEWEITGQPEPENNYQTTPPQLDSDGTYFLYSRLSVDRSHWQRGNTYTCSVSHEALHSHHTQKSLTQSPGK

SEQ ID NO: 40 - DNA, 1290 bp, Felis catus
  source 1..1290: mol_type = other DNA
atggatggacctagacctctgctgctgctcctgcctctgctgttgggagtttctctcggcggagccaaagaggcttgtcctaccggcctgtttacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagatccctggaagaggcggaggcggagtgcctaagaccgcttctaccatcgagtccaagacctgcgactgccctaagtgccctgtgcctgaaattcctggcgctccctccgtgttcatcttcccacctaagcctaaggacaccctgtccatctctcggacccctgaagtgacctgcctggtggttgatctgggccctgacgactccaacgtgcagatcacttggtttgtggacaacaccgagatgcacaccgccaagaccagacctagagaggaacagttcaactccacctacagagtggtgtccgtgctgcccatcctgcaccaggattggctgaagggcaaagaattcaagtgcaaagtgaactccaagagcctgccttccgccatggaacggaccatctctaaggctaagggccagcctcatgagccccaggtgtacgttctgcctcctacacaagaggaactgtccgagaacaaagtgtccgtgacatgcctgatcaagggctttcaccctcctgatatcgccgtggaatgggagatcaccggacagcctgagcctgagaacaactaccagaccacacctcctcagctggacagcgacggaacctacttcctgtactcccggctgtccgtggacagatcccattggcagagaggcaacacctacacctgttccgtgtctcacgaggccctgcactctcatcacacccagaagtccctgacacagtcccctggcaag

SEQ ID NO: 41 - protein, 229 aa, Felis catus
LPPCKCPKCPVPEIPGGPSVFIFPPKPKDTLSISRTPEVTCLVVDLGPDDSNVQITWFVDNTEMHTAKTRPREEQFNSTYRVVSVLPIVHQDWLTGKEFKCKVNSKALPSAIERTISKAKGQPHEPQVYVLPPAQEELSENKVCVTCLIKGFYPPDIAVEWEITGQPEPENNYRTTPPQLDSDGTYFVYSRLSMDRSRWQSGNTYTCSVSHEALHSHHTQKSLTQSPGK

SEQ ID NO: 42 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 43 - DNA, 1290 bp, Felis catus
  source 1..1290: mol_type = genomic DNA
atggatggccctagacctctgctgctgctgttgcctctgctcctgggagtttctctcggcggagccaaagaggcttgtcctaccggcctgtttacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagattcctggcggaggcggagtgcgcaagacagatcatcctcctggacctaagccttgcgactgccctaagtgtcccgctcctgaaatgctcggcggacccagcatcttcatcttcccacctaagccaaaggacaccctgtccatctctcggacccctgaagtgacctgcctggtggttgatctgggccctgacgattccgacgtgcagatcacttggtttgtggacaacacccaggtgtacacagccaagacctctccaagagaggaacagttcaactccacctacagagtggtgtccgtgctgcccatcctgcaccaggattggctgaagggcaaagaattcaagtgcaaagtgaactccaagagcctgccttctccaatcgagcggaccatctccaaggctaagggccagcctcatgagcctcaggtgtacgttctgcctcctgctcaagaggaactgtcccggaacaaagtgtctgtgacctgtctgatcaagagctttcaccctcctgatatcgccgtggaatgggagatcaccggacagcctgagcctgagaacaactaccggaccacacctcctcagctggacagcgacggcacatacttcgtgtactccaagctgtccgtggacagatcccactggcagcggggcaatacctacacctgttccgtgtctcacgaggccctgcactctcatcacacccagaagtccctgacacagtcccctggaaagtgatga

SEQ ID NO: 44 - protein, 420 aa, Felis catus
  SIGNAL 1..22
  REGION 191..420: note = Feline Fc-IgG3
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPGGGGVLPPCKCPKCPVPEIPGGPSVFIFPPKPKDTLSISRTPEVTCLVVDLGPDDSNVQITWFVDNTEMHTAKTRPREEQFNSTYRVVSVLPIVHQDWLTGKEFKCKVNSKALPSAIERTISKAKGQPHEPQVYVLPPAQEELSENKVCVTCLIKGFYPPDIAVEWEITGQPEPENNYRTTPPQLDSDGTYFVYSRLSMDRSRWQSGNTYTCSVSHEALHSHHTQKSLTQSPGK

SEQ ID NO: 45 - DNA, 1260 bp, Felis catus
  source 1..1260: mol_type = genomic DNA
atggatggccctagacctctgctgctgctgttgcctctgctcctgggagtttctctcggcggagccaaagaggcttgtcctaccggcctgtttacccactctggcgagtgttgcaaggcctgtaatctcggcgaaggcgtggcacaaccttgtggcgctaatcagacagtgtgcgagccttgcctggactccgtgaccttctctgatgtggtgtctgccaccgagccatgcaagccttgtaccgagtgtgtgggcctgcagtccatgtctgccccttgtgtggaagccgacgacgccgtgtgtagatgtgcctacggctactaccaggacgagacaaccggaagatgcgaggcctgcagagtgtgtgaagctggctctggactggtgttctcctgccaagacagacagaacaccgtgtgcgaggaatgccctgacggcacctactctgatgaggccaatcacgtggacccctgcctgccttgtactgtgtgcgaagataccgagcggcagctgcgcgagtgtaccagatgggctgatgccgagtgcgaagagattcctggcggaggcggagttctgcctccttgcaagtgtcctaagtgccccgtgcctgaaatccctggcggcccttccgtgttcatcttcccacctaagcctaaggacaccctgtccatctctcggacccctgaagtgacctgcctggtggttgatctgggccctgacgactccaacgtgcagatcacttggtttgtggacaacaccgagatgcacaccgccaagaccagacctagagaggaacagttcaactccacctacagagtggtgtccgtgctgcccatcgtgcaccaggattggctgaccggcaaagaattcaagtgcaaagtgaacagcaaggccctgccttccgccatcgagcggacaatctctaaggctaagggccagcctcacgagccccaggtttacgttttgcctcctgctcaagaggaactgtccgagaacaaagtgtgcgtgacctgtctgatcaagggcttctaccctcctgatatcgccgtggaatgggagatcaccggacagcctgagcctgagaacaactaccggaccacacctcctcagctggattccgacggcacatacttcgtgtactcccggctgagcatggacagatccagatggcagtccggcaacacctacacctgttccgtgtctcacgaggccctgcactctcatcacacccagaagtccctgacacagtcccctggcaag

SEQ ID NO: 46 - protein, 166 aa, Canis lupus
  source 1..166: note = subspecies familiaris
KEACPTGLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDRQNTVCEECPDGTYSDEANHVDPCLPCTVCEDTERQLRECTRWADAECEEIPGR

SEQ ID NO: 47 - protein, 424 aa, Canis lupus
  source 1..424: note = subspecies familiaris
  SIGNAL 1..19
  REGION 190..424: note = Canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDRQNTVCEECPDGTYSDEANHVDPCLPCTVCEDTERQLRECTRWADAECEEIPGRGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 48 - DNA, 1272 bp, Canis lupus
  source 1..1272: mol_type = other DNA; sub_species = familiaris
atggaatggagctgggtgtttctgttttttctgagcgtgaccaccggcgtgcatagcaaagaagcgtgcccgaccggcctgtatacccatagcggcgaatgctgcaaagcgtgcaacctgggcgaaggcgtggcgcagccgtgcggcgcgaaccagaccgtgtgcgaaccgtgcctggatagcgtgacctttagcgatgtggtgagcgcgaccgaaccgtgcaaaccgtgcaccgaatgcgtgggcctgcagagcatgagcgcgccgtgcgtggaagcgaccgatgcggtgtgccgctgcgcgtatggctattatcaggatgaaaccaccggccgctgcgaagcgtgccgcgtgtgcgaagcgggcagcggcctggtgtttagctgccaggatcgccagaacaccgtgtgcgaagaatgcccggatggcacctatagcgatgaagcgaaccatgtggatccgtgcctgccgtgcaccgtgtgcgaagataccgaacgccagctgcgcgaatgcacccgctgggcggatgcggaatgcgaagaaattccgggccgcggcggcggcggccgcgaaaacggccgcgtgccgcgcccgccggattgcccgaaatgcccggcgccggaaatgctgggcggcccgagcgtgtttatttttccgccgaaaccgaaagataccctgctgattgcgcgcaccccggaagtgacctgcgtggtggtggatctggatccggaagatccggaagtgcagattagctggtttgtggatggcaaacagatgcagaccgcgaaaacccagccgcgcgaagaacagtttaacggcacctatcgcgtggtgagcgtgctgccgattggccatcaggattggctgaaaggcaaacagtttacctgcaaagtgaacaacaaagcgctgccgagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccagaaaagcctgagccatagcccgggcaaa

SEQ ID NO: 49 - protein, 166 aa, Canis lupus
  source 1..166: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDRQNTVCEECPDGTYSDEANHVDPCLPCTVCEDTERQLRECTRWADAECEEIPGR

SEQ ID NO: 50 - protein, 424 aa, Canis lupus
  source 1..424: note = subspecies familiaris
  SIGNAL 1..19
  REGION 190..424: note = Canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDRQNTVCEECPDGTYSDEANHVDPCLPCTVCEDTERQLRECTRWADAECEEIPGRGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 51 - DNA, 1272 bp, Canis lupus
  source 1..1272: mol_type = other DNA; sub_species = familiaris
atggaatggagctgggtgtttctgttttttctgagcgtgaccaccggcgtgcatagcaaagaagcgtgcccgaccggcctgtatacccatagcggcgaatgctgcaaagcgtgcaacctgggcgaaggcgtggcgcagccgtgcggcgcgaaccagaccgtgtgcgaaccgtgcctggatagcgtgacctttagcgatgtggtgagcgcgaccgaaccgtgcaaaccgtgcaccgaatgcgtgggcctgcagagcatgagcgcgccgtgcgtggaagcggatgatgcggtgtgccgctgcgcgtatggctattatcaggatgaaaccaccggccgctgcgaagcgtgccgcgtgtgcgaagcgggcagcggcctggtgttttattgccaggatcgccagaacaccgtgtgcgaagaatgcccggatggcacctatagcgatgaagcgaaccatgtggatccgtgcctgccgtgcaccgtgtgcgaagataccgaacgccagctgcgcgaatgcacccgctgggcggatgcggaatgcgaagaaattccgggccgcggcggcggcggccgcgaaaacggccgcgtgccgcgcccgccggattgcccgaaatgcccggcgccggaaatgctgggcggcccgagcgtgtttatttttccgccgaaaccgaaagataccctgctgattgcgcgcaccccggaagtgacctgcgtggtggtggatctggatccggaagatccggaagtgcagattagctggtttgtggatggcaaacagatgcagaccgcgaaaacccagccgcgcgaagaacagtttaacggcacctatcgcgtggtgagcgtgctgccgattggccatcaggattggctgaaaggcaaacagtttacctgcaaagtgaacaacaaagcgctgccgagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccagaaaagcctgagccatagcccgggcaaa

SEQ ID NO: 52 - protein, 166 aa, Canis lupus
  source 1..166: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDRQNTVCEECPDGTYSDEANHVDPCLPCTVCEDTERQLRECTRWADAECEEIPGR

SEQ ID NO: 53 - protein, 424 aa, Canis lupus
  source 1..424: note = subspecies familiaris
  SIGNAL 1..19
  REGION 190..424: note = Canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDRQNTVCEECPDGTYSDEANHVDPCLPCTVCEDTERQLRECTRWADAECEEIPGRGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 54 - DNA, 1272 bp, Canis lupus
  source 1..1272: mol_type = other DNA; sub_species = familiaris
atggaatggagctgggtgtttctgttttttctgagcgtgaccaccggcgtgcatagcaaagaagcgtgcccgaccggcctgtatacccatagcggcgaatgctgcaaagcgtgcaacctgggcgaaggcgtggcgcagccgtgcggcgcgaaccagaccgtgtgcgaaccgtgcctggatagcgtgacctttagcgatgtggtgagcgcgaccgaaccgtgcaaaccgtgcaccgaatgcgtgggcctgcagagcatgagcgcgccgtgcgtggaagcggatgatgcggtgtgccgctgcgcgtatggctattatcaggatgaaaccaccggccgctgcgaagcgtgccgcgtgtgcgaagcgggcagcggcctggtgtttcattgccaggatcgccagaacaccgtgtgcgaagaatgcccggatggcacctatagcgatgaagcgaaccatgtggatccgtgcctgccgtgcaccgtgtgcgaagataccgaacgccagctgcgcgaatgcacccgctgggcggatgcggaatgcgaagaaattccgggccgcggcggcggcggccgcgaaaacggccgcgtgccgcgcccgccggattgcccgaaatgcccggcgccggaaatgctgggcggcccgagcgtgtttatttttccgccgaaaccgaaagataccctgctgattgcgcgcaccccggaagtgacctgcgtggtggtggatctggatccggaagatccggaagtgcagattagctggtttgtggatggcaaacagatgcagaccgcgaaaacccagccgcgcgaagaacagtttaacggcacctatcgcgtggtgagcgtgctgccgattggccatcaggattggctgaaaggcaaacagtttacctgcaaagtgaacaacaaagcgctgccgagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccagaaaagcctgagccatagcccgggcaaa

SEQ ID NO: 55 - protein, 166 aa, Canis lupus
  source 1..166: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHRDPCLPCTVCEDTERQLRECTRWADAECEEIPGR

SEQ ID NO: 56 - protein, 424 aa, Canis lupus
  source 1..424: note = subspecies familiaris
  SIGNAL 1..19
  REGION 190..424: note = Canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHRDPCLPCTVCEDTERQLRECTRWADAECEEIPGRGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 57 - DNA, 1272 bp, Canis lupus
  source 1..1272: mol_type = other DNA; sub_species = familiaris
atggaatggagctgggtgtttctgttttttctgagcgtgaccaccggcgtgcatagcaaagaagcgtgcccgaccggcctgtatacccatagcggcgaatgctgcaaagcgtgcaacctgggcgaaggcgtggcgcagccgtgcggcgcgaaccagaccgtgtgcgaaccgtgcctggatagcgtgacctttagcgatgtggtgagcgcgaccgaaccgtgcaaaccgtgcaccgaatgcgtgggcctgcagagcatgagcgcgccgtgcgtggaagcggatgatgcggtgtgccgctgcgcgtatggctattatcaggatgaaaccaccggccgctgcgaagcgtgccgcgtgtgcgaagcgggcagcggcctggtgtttagctgccaggatcgccagaacaccgtgtgcgaagaatgcccggatggcacctatagcgatgaagcgaaccatcgcgatccgtgcctgccgtgcaccgtgtgcgaagataccgaacgccagctgcgcgaatgcacccgctgggcggatgcggaatgcgaagaaattccgggccgcggcggcggcggccgcgaaaacggccgcgtgccgcgcccgccggattgcccgaaatgcccggcgccggaaatgctgggcggcccgagcgtgtttatttttccgccgaaaccgaaagataccctgctgattgcgcgcaccccggaagtgacctgcgtggtggtggatctggatccggaagatccggaagtgcagattagctggtttgtggatggcaaacagatgcagaccgcgaaaacccagccgcgcgaagaacagtttaacggcacctatcgcgtggtgagcgtgctgccgattggccatcaggattggctgaaaggcaaacagtttacctgcaaagtgaacaacaaagcgctgccgagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccagaaaagcctgagccatagcccgggcaaa

SEQ ID NO: 58 - protein, 166 aa, Canis lupus
  source 1..166: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVLPCLPCTVCEDTERQLRECTRWADAECEEIPGR

SEQ ID NO: 59 - protein, 424 aa, Canis lupus
  source 1..424: note = subspecies familiaris
  SIGNAL 1..19
  REGION 190..424: note = Canine Fc-B
MEWSWVFLFFLSVTTGVHSKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVLPCLPCTVCEDTERQLRECTRWADAECEEIPGRGGGGRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 60 - DNA, 1271 bp, Canis lupus
  source 1..1271: mol_type = other DNA; sub_species = familiaris
atggaatggagctgggtgtttctgttttttctgagcgtgaccaccggcgtgcatagcaaagaagcgtgcccgaccggcctgtatacccatagcggcgaatgctgcaaagcgtgcaacctgggcgaaggcgtggcgcagccgtgcggcgcgaaccagaccgtgtgcgaaccgtgcctggatagcgtgacctttagcgatgtggtgagcgcgaccgaaccgtgcaaaccgtgcaccgaatcgtgggcctgcagagcatgagcgcgccgtgcgtggaagcggatgatgcggtgtgccgctgcgcgtatggctattatcaggatgaaaccaccggccgctgcgaagcgtgccgcgtgtgcgaagcgggcagcggcctggtgtttagctgccaggatcgccagaacaccgtgtgcgaagaatgcccggatggcacctatagcgatgaagcgaaccatgtgctgccgtgcctgccgtgcaccgtgtgcgaagataccgaacgccagctgcgcgaatgcacccgctgggcggatgcggaatgcgaagaaattccgggccgcggcggcggcggccgcgaaaacggccgcgtgccgcgcccgccggattgcccgaaatgcccggcgccggaaatgctgggcggcccgagcgtgtttatttttccgccgaaaccgaaagataccctgctgattgcgcgcaccccggaagtgacctgcgtggtggtggatctggatccggaagatccggaagtgcagattagctggtttgtggatggcaaacagatgcagaccgcgaaaacccagccgcgcgaagaacagtttaacggcacctatcgcgtggtgagcgtgctgccgattggccatcaggattggctgaaaggcaaacagtttacctgcaaagtgaacaacaaagcgctgccgagcccgattgaacgcaccattagcaaagcgcgcggccaggcgcatcagccgagcgtgtatgtgctgccgccgagccgcgaagaactgagcaaaaacaccgtgagcctgacctgcctgattaaagatttttttccgccggatattgatgtggaatggcagagcaacggccagcaggaaccggaaagcaaatatcgcaccaccccgccgcagctggatgaagatggcagctattttctgtatagcaaactgagcgtggataaaagccgctggcagcgcggcgatacctttatttgcgcggtgatgcatgaagcgctgcataaccattatacccagaaaagcctgagccatagcccgggcaaa

SEQ ID NO: 61 - protein, 164 aa, Felis catus
KEACPTGLFTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDRQNTVCEECPDGTYSDEANHVDPCLPCTVCEDTERQLRECTRWADAECEEIP

SEQ ID NO: 62 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDRQNTVCEECPDGTYSDEANHVDPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 63 - protein, 164 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDRQNTVCEECPDGTYSDEANHVDPCLPCTVCEDTERQLRECTRWADAECEEIP

SEQ ID NO: 64 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDRQNTVCEECPDGTYSDEANHVDPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 65 - protein, 164 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDRQNTVCEECPDGTYSDEANHVDPCLPCTVCEDTERQLRECTRWADAECEEIP

SEQ ID NO: 66 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDRQNTVCEECPDGTYSDEANHVDPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 67 - protein, 164 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHRDPCLPCTVCEDTERQLRECTRWADAECEEIP

SEQ ID NO: 68 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHRDPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 69 - protein, 164 aa, Felis catus
KEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVLPCLPCTVCEDTERQLRECTRWADAECEEIP

SEQ ID NO: 70 - protein, 428 aa, Felis catus
  SIGNAL 1..22
  REGION 191..428: note = Feline Fc-IgG1
MDGPRPLLLLLPLLLGVSLGGAKEACPTGLFTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVLPCLPCTVCEDTERQLRECTRWADAECEEIPGGGGVRKTDHPPGPKPCDCPKCPAPEMLGGPSIFIFPPKPKDTLSISRTPEVTCLVVDLGPDDSDVQITWFVDNTQVYTAKTSPREEQFNSTYRVVSVLPILHQDWLKGKEFKCKVNSKSLPSPIERTISKAKGQPHEPQVYVLPPAQEELSRNKVSVTCLIKSFHPPDIAVEWEITGQPEPENNYRTTPPQLDSDGTYFVYSKLSVDRSHWQRGNTYTCSVSHEALHSHHTQKSLTQSPGK

SEQ ID NO: 71 - protein, 427 aa, Homo sapiens
  SIGNAL 1..28
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 72 - DNA, 1281 bp, Homo sapiens
  source 1..1281: mol_type = other DNA
atgggcgccggcgccaccggcagggccatggacggccccaggctgctgctgctgctgctgctgggcgtgagcctgggcggcgccaaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttcagctgccaggacaagcagaacaccgtgtgcgaggagtgccccgacggcacctacagcgacgaggccaaccacgtggacccctgcctgccctgcaccgtgtgcgaggacaccgagaggcagctgagggagtgcaccaggtgggccgacgccgagtgcgaggagatccccggcaggtggatcaccaggagcaccccccccgagggcagcgacagcaccgcccccagcacccaggagcccgaggccccccccgagcaggacctgatcgccagcaccgtggccggcgtggtgaccaccgtgatgggcagcagccagcccgtggtgaccaggggcaccaccgacaacctgatccccgtgtactgcagcatcctggccgccgtggtggtgggcctggtggcctacatcgccttcaagaggtggaacagctgcaagcagaacaagcagggcgccaacagcaggcccgtgaaccagacccccccccccgagggcgagaagctgcacagcgacagcggcatcagcgtggacagccagagcctgcacgaccagcagccccacacccagaccgccagcggccaggccctgaagggcgacggcggcctgtacagcagcctgccccccgccaagagggaggaggtggagaagctgctgaacggcagcgccggcgacacctggaggcacctggccggcgagctgggctaccagcccgagcacatcgacagcttcacccacgaggcctgccccgtgagggccctgctggccagctgggccacccaggacagcgccaccctggacgccctgctggccgccctgaggaggatccagagggccgacctggtggagagcctgtgcagcgagagcaccgccaccagccccgtg

SEQ ID NO: 73 - protein, 208 aa, Homo sapiens
  REGION 165..192: note = stalk region
  REGION 193..208: note = alpha and gamma secretase sites
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVM

SEQ ID NO: 74 - DNA, 624 bp, Homo sapiens
  source 1..624: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttcagctgccaggacaagcagaacaccgtgtgcgaggagtgccccgacggcacctacagcgacgaggccaaccacgtggacccctgcctgccctgcaccgtgtgcgaggacaccgagaggcagctgagggagtgcaccaggtgggccgacgccgagtgcgaggagatccccggcaggtggatcaccaggagcaccccccccgagggcagcgacagcaccgcccccagcacccaggagcccgaggccccccccgagcaggacctgatcgccagcaccgtggccggcgtggtgaccaccgtgatg

SEQ ID NO: 75 - protein, 44 aa, Homo sapiens
  REGION 1..28: note = stalk region
  REGION 29..44: note = alpha and gamma secretase cleavage sites
GRWITRSTPPEGSDSTAPSTQEPEAPPEQDLIASTVAGVVTTVM

SEQ ID NO: 76 - DNA, 132 bp, Homo sapiens
  source 1..132: mol_type = other DNA
ggcaggtggatcaccaggagcaccccccccgagggcagcgacagcaccgcccccagcacccaggagcccgaggccccccccgagcaggacctgatcgccagcaccgtggccggcgtggtgaccaccgtgatg

SEQ ID NO: 77 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 79 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 80 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 444 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 411 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 400 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 164 aa, Homo sapiens
KEACPTGLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDKQNTVCEECPDGTYSDEANHVDPCLPCTVCEDTERQLRECTRWADAECEEIP

SEQ ID NO: 85 - protein, 400 aa, Homo sapiens
KEACPTGLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDKQNTVCEECPDGTYSDEANHVDPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - DNA, 1199 bp, Homo sapiens
  source 1..1199: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccaccgacgccgtgtgcggtgcgcctacggctactaccaggacgagaccaccggcaggtgcgaggcctgcagggtgtgcgaggccggcagcggcctggtgttcagctgccaggacaagcagaacaccgtgtgcgaggagtgccccgacggcacctacagcgacgaggccaaccacgtggacccctgcctgccctgcaccgtgtgcgaggacaccgagaggcagctgagggagtgcaccaggtgggccgacgccgagtgcgaggagatccccggcggcggcggcgagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 87 - protein, 164 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDKQNTVCEECPDGTYSDEANHVDPCLPCTVCEDTERQLRECTRWADAECEEIP

SEQ ID NO: 88 - protein, 400 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDKQNTVCEECPDGTYSDEANHVDPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1200 bp, Homo sapiens
  source 1..1200: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttctactgccaggacaagcagaacaccgtgtgcgaggagtgccccgacggcacctacagcgacgaggccaaccacgtggacccctgcctgccctgcaccgtgtgcgaggacaccgagaggcagctgagggagtgcaccaggtgggccgacgccgagtgcgaggagatccccggcggcggcggcgagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 90 - protein, 164 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDKQNTVCEECPDGTYSDEANHVDPCLPCTVCEDTERQLRECTRWADAECEEIP

SEQ ID NO: 91 - protein, 400 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDKQNTVCEECPDGTYSDEANHVDPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - DNA, 1200 bp, Homo sapiens
  source 1..1200: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttccactgccaggacaagcagaacaccgtgtgcgaggagtgccccgacggcacctacagcgacgaggccaaccacgtggacccctgcctgccctgcaccgtgtgcgaggacaccgagaggcagctgagggagtgcaccaggtgggccgacgccgagtgcgaggagatccccggcggcggcggcgagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 93 - protein, 164 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHRDPCLPCTVCEDTERQLRECTRWADAECEEIP

SEQ ID NO: 94 - protein, 400 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHRDPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1200 bp, Homo sapiens
  source 1..1200: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttcagctgccaggacaagcagaacaccgtgtgcgaggagtgccccgacggcacctacagcgacgaggccaaccacagggacccctgcctgccctgcaccgtgtgcgaggacaccgagaggcagctgagggagtgcaccaggtgggccgacgccgagtgcgaggagatccccggcggcggcggcgagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 96 - protein, 164 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVLPCLPCTVCEDTERQLRECTRWADAECEEIP

SEQ ID NO: 97 - protein, 400 aa, Homo sapiens
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVLPCLPCTVCEDTERQLRECTRWADAECEEIPGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1200 bp, Homo sapiens
  source 1..1200: mol_type = other DNA
aaggaggcctgccccaccggcctgtacacccacagcggcgagtgctgcaaggcctgcaacctgggcgagggcgtggcccagccctgcggcgccaaccagaccgtgtgcgagccctgcctggacagcgtgaccttcagcgacgtggtgagcgccaccgagccctgcaagccctgcaccgagtgcgtgggcctgcagagcatgagcgccccctgcgtggaggccgacgacgccgtgtgcaggtgcgcctacggctactaccaggacgagaccaccggcaggtgcgaggcctgcagggtgtgcgaggccggcagcggcctggtgttcagctgccaggacaagcagaacaccgtgtgcgaggagtgccccgacggcacctacagcgacgaggccaaccacgtgctgccctgcctgccctgcaccgtgtgcgaggacaccgagaggcagctgagggagtgcaccaggtgggccgacgccgagtgcgaggagatccccggcggcggcggcgagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 99 - protein, 427 aa, Camelus dromedarius
  SIGNAL 1..28
MGAGAAGCAMDGPRLLLLLLLGVSLGGAKESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRATPPEGSFSTDPSTQEPEVPPDQELVTSTVSDVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTAAGQALKGDGGLYSSLPLAKREEVEKLLNGSAGDTWRHLAGELGYQPEHIDSFTREACPVRALLASWATQDSATLDTLLAALRRIQRADIVESLCSESTATSPV

SEQ ID NO: 100 - DNA, 1284 bp, Camelus dromedarius
  source 1..1284: mol_type = genomic DNA
atgggggcaggtgccgccggctgcgccatggacgggccgcgcctgcttttgctgctgctcctgggggtgtcccttggaggtgccaaagagtcatgccccacggacctgtacacccacagcggcgagtgctgcaaagcctgcaacctgggcgagggcgtggcccagccttgtggagccaaccagaccgtgtgtgagccctgcctggacagcgtgaccttttcggacgtggtgagcgctaccgagccgtgtaagccgtgcaccgagtgcgtggggctgcagagcatgtcggcgccgtgcgtggaggccgacgatgccgtgtgccgctgcgcctacggctactaccaggacgagacgaccggccgctgcgaggcgtgccgcgtgtgcgaggcgggctcggggctcgtgttctcctgccaggacaagcagaacacggtgtgcgaggagtgccccgacggcacctactccgacgaggccaaccacgtggacccgtgcctgccctgcacggtgtgcgaggacaccgagcgccagctgcgcgagtgcacgcgctgggccgacgccgagtgcgaggagatccctggccgttggatcacacgggccacacccccggagggctcattcagcacagaccccagcacccaagagcctgaggtacctccagaccaagagcttgtaaccagcacggtgtccgatgtggtgactacagtgatgggcagctcccagcctgtggtgacccgaggtaccaccgacaacctcatcccggtctattgctccatcctggctgctgtggttgtgggcctcgtggcctacatcgccttcaagaggtggaacagctgcaagcagaacaagcaaggagccaacagccggcctgtgaaccagacgcccccacccgagggagaaaaactccacagcgacagcggcatctccgtggacagccagagcctgcatgaccagcagccccacacgcagacggctgcaggccaggccctcaagggtgatggaggactctacagcagcctgccactggccaagcgggaggaggtggagaagctgctcaatggctctgcgggggacacctggcggcacctggcgggcgagctgggctaccagcccgaacacatagactccttcacccgtgaggcctgcccggtccgcgccctgctggctagctgggccacccaggacagcgccacgcttgacaccctcctggctgccttgcgccgtatccagcgagctgacattgtggagagcctgtgcagcgagtccacggccacatccccggtgtga

SEQ ID NO: 101 - protein, 208 aa, Camelus dromedarius
  REGION 165..192: note = stalk region
  REGION 193..208: note = alpha and gamma secretase sites
KESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRATPPEGSFSTDPSTQEPEVPPDQELVTSTVSDVVTTVM

SEQ ID NO: 102 - DNA, 624 bp, Camelus dromedarius
  source 1..624: mol_type = other DNA
aaagagtcatgccccacggacctgtacacccacagcggcgagtgctgcaaagcctgcaacctgggcgagggcgtggcccagccttgtggagccaaccagaccgtgtgtgagccctgcctggacagcgtgaccttttcggacgtggtgagcgctaccgagccgtgtaagccgtgcaccgagtgcgtggggctgcagagcatgtcggcgccgtgcgtggaggccgacgatgccgtgtgccgctgcgcctacggctactaccaggacgagacgaccggccgctgcgaggcgtgccgcgtgtgcgaggcgggctcggggctcgtgttctcctgccaggacaagcagaacacggtgtgcgaggagtgccccgacggcacctactccgacgaggccaaccacgtggacccgtgcctgccctgcacggtgtgcgaggacaccgagcgccagctgcgcgagtgcacgcgctgggccgacgccgagtgcgaggagatccctggccgttggatcacacgggccacacccccggagggctcattcagcacagaccccagcacccaagagcctgaggtacctccagaccaagagcttgtaaccagcacggtgtccgatgtggtgactacagtgatg

SEQ ID NO: 103 - protein, 164 aa, Camelus dromedarius
KESCPTDLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDKQNTVCEECPDGTYSDEANHVDPCLPCTVCEDTERQLRECTRWADAECEEIP

SEQ ID NO: 104 - protein, 164 aa, Camelus dromedarius
KESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDKQNTVCEECPDGTYSDEANHVDPCLPCTVCEDTERQLRECTRWADAECEEIP

SEQ ID NO: 105 - protein, 164 aa, Camelus dromedarius
KESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDKQNTVCEECPDGTYSDEANHVDPCLPCTVCEDTERQLRECTRWADAECEEIP

SEQ ID NO: 106 - protein, 164 aa, Camelus dromedarius
KESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHRDPCLPCTVCEDTERQLRECTRWADAECEEIP

SEQ ID NO: 107 - protein, 164 aa, Camelus dromedarius
KESCPTDLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVLPCLPCTVCEDTERQLRECTRWADAECEEIP

SEQ ID NO: 108 - protein, 428 aa, Sus scrofa
  SIGNAL 1..29
MGAGAAGCAMDWPRLLLLLLLQGVSLGGAKETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWMTRATPAEGSDSTEPSTQEPEVPPEQDFVTSTVSDVVTTVMGSSQPVVTRGTTDNLIPVYCSILAAVVMGLVAYIAFKRWNSCKQNKQGANSRPVNQTPPPEGEKLHSDSGISVDSQSLHDQQPHTQTAASQGLKGDGGLYSSLPLAKREEVEKLLNGSAGDTWRHLAGELGYQPEHIDSFTHEACPVRALLASWAAQDSATLDTLLAALRRIQRADIVESLCSESTATSPV

SEQ ID NO: 109 - DNA, 1287 bp, Sus scrofa
  source 1..1287: mol_type = genomic DNA
atgggggcaggtgccgccggctgcgccatggactggccgcgcctgctgctgctgctgctgctccagggggtgtcccttggaggtgccaaggagacctgtcccacaggcatgtacactcacagcggcgagtgctgcaaagcctgcaacctgggtgaaggcgtggcccagccttgcggagccaaccagaccgtgtgtgagccctgcctggacagcgtgaccttctcggacgtggtgagcgccaccgagccgtgtaagccgtgcaccgagtgcgtggggctgcagagcatgtcagcgccctgcgtggaggcggacgacgccgtgtgccgctgcgcctacggctactaccaggacgagacgaccggccgctgcgaggcgtgtcgcgtgtgcgaggcgggctcggggctggtgttctcgtgccaggataagcagaacaccgtgtgcgaggagtgccccgacggcacgtactccgacgaggccaaccacgtggacccgtgcctgccctgcacggtgtgcgaggacaccgagcgccagcttcgcgagtgcacgcgctgggccgacgcggagtgcgaggagatccctggccgctggatgacacgggctacacccgccgagggctccgacagcacagagcccagcacccaggagcctgaggtacctccagagcaagactttgtaaccagcacggtgtccgatgtggtgaccacagtgatggggagctcccagcccgtggtgacccgaggcaccaccgacaacctcatccctgtctattgctccatcctggctgccgtggtcatgggcctggtggcctacatcgccttcaagaggtggaacagctgcaagcagaacaagcaaggagccaacagccggcccgtgaaccagacgccgccacccgagggagagaaactccacagtgacagtggcatctctgtggacagccagagcctgcatgaccagcagccccacacgcagacggccgcaagccagggcctcaagggtgacggaggcctctacagcagcctgccgctggccaagcgggaggaggtggagaagctgctgaatggctccgcaggagacacctggcggcacctggcgggcgagctgggctaccagcccgagcacatagactccttcacccacgaggcctgcccggtccgcgccctgctggccagctgggccgcccaggacagcgccacgctcgacaccctcctggccgccctgcgccgtatccagcgagccgacatcgtggagagcctgtgcagcgagtccacggccacgtccccagtgtga

SEQ ID NO: 110 - protein, 208 aa, Sus scrofa
  REGION 165..192: note = stalk region
  REGION 193..208: note = alpha and gamma secretase sites
KETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWMTRATPAEGSDSTEPSTQEPEVPPEQDFVTSTVSDVVTTVM

SEQ ID NO: 111 - DNA, 624 bp, Sus scrofa
  source 1..624: mol_type = other DNA
aaggagacctgtcccacaggcatgtacactcacagcggcgagtgctgcaaagcctgcaacctgggtgaaggcgtggcccagccttgcggagccaaccagaccgtgtgtgagccctgcctggacagcgtgaccttctcggacgtggtgagcgccaccgagccgtgtaagccgtgcaccgagtgcgtggggctgcagagcatgtcagcgccctgcgtggaggcggacgacgccgtgtgccgctgcgcctacggctactaccaggacgagacgaccggccgctgcgaggcgtgtcgcgtgtgcgaggcgggctcggggctggtgttctcgtgccaggataagcagaacaccgtgtgcgaggagtgccccgacggcacgtactccgacgaggccaaccacgtggacccgtgcctgccctgcacggtgtgcgaggacaccgagcgccagcttcgcgagtgcacgcgctgggccgacgcggagtgcgaggagatccctggccgctggatgacacgggctacacccgccgagggctccgacagcacagagcccagcacccaggagcctgaggtacctccagagcaagactttgtaaccagcacggtgtccgatgtggtgaccacagtgatg

SEQ ID NO: 112 - protein, 164 aa, Sus scrofa
KETCPTGMYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDKQNTVCEECPDGTYSDEANHVDPCLPCTVCEDTERQLRECTRWADAECEEIP

SEQ ID NO: 113 - protein, 164 aa, Sus scrofa
KETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDKQNTVCEECPDGTYSDEANHVDPCLPCTVCEDTERQLRECTRWADAECEEIP

SEQ ID NO: 114 - protein, 164 aa, Sus scrofa
KETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDKQNTVCEECPDGTYSDEANHVDPCLPCTVCEDTERQLRECTRWADAECEEIP

SEQ ID NO: 115 - protein, 164 aa, Sus scrofa
KETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHRDPCLPCTVCEDTERQLRECTRWADAECEEIP

SEQ ID NO: 116 - protein, 164 aa, Sus scrofa
KETCPTGMYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVLPCLPCTVCEDTERQLRECTRWADAECEEIP

SEQ ID NO: 117 - protein, 328 aa, Canis lupus
  source 1..328: note = subspecies familiaris
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDRQNTVCEECPDGTYSDEANHVDPCLPCTVCEDTERQLRECTRWADAECEEIP

SEQ ID NO: 118 - protein, 208 aa, Equus caballus
  REGION 165..192: note = stalk region
  REGION 193..208: note = alpha and gamma secretase sites
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFSCQDKQNTVCEECPDGTYSDEANHVDPCLPCTVCEDTERQLRECTRWADAECEEIPSRWITRATPPEGSDSTAPSTQEPEGPPEKDLVASTVADVVTTVM

SEQ ID NO: 119 - DNA, 624 bp, Equus caballus
  source 1..624: mol_type = other DNA
atgagggcaggtgccgccgactgcgccatggacggaccgcgccttctgctgctgcttctgctcttgggggtgtgcctgctgggaggtgccaaggaggtgtgccccacagacctgtacacccacagcggcgagtgctgcaaagcctgcaacctgggcgagggtgtggcccagccttgcggagccaaccagactgtgtgtgaaccctgcctggacagcgtgacgttctcggacgtggtgagcgccacagagccatgtaagccgtgcaccgagtgcgtgggcctgcagagcatgtcggcgccatgcgtggaggccgacgacgcggtgtgccgctgcgcctatggctactaccaggacgagacgacgggccgctgcgaggcgtgccaggtgtgcgaggcgggctcgggcctcgtgttctcgtgccaggacaagcagaacaccgtgtgcgaggaatgccccgacggcacgtactccgacgaggccaaccacgtggacccgtgcctgccctgcaccgtgtgcgaggacaccgagcgccagctgcgagagtgcacgcgctgggccgacgccgagtgcgaggagatccccagccgttggattacacgggccacgccgccggagggctcagac

SEQ ID NO: 120 - protein, 164 aa, Equus caballus
KEVCPTDLYTHSGECCKACNLGEGVAQPCGANQTVCEPCLDSVTFSDVVSATEPCKPCTECVGLQSMSAPCVEATDAVCRCAYGYYQDETTGRCEACQVCEAGSGLVFSCQDKQNTVCEECPDGTYSDEANHVDPCLPCTVCEDTERQLRECTRWADAECEEIP

SEQ ID NO: 121 - protein, 164 aa, Equus caballus
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFYCQDKQNTVCEECPDGTYSDEANHVDPCLPCTVCEDTERQLRECTRWADAECEEIP

SEQ ID NO: 122 - protein, 164 aa, Equus caballus
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFHCQDKQNTVCEECPDGTYSDEANHVDPCLPCTVCEDTERQLRECTRWADAECEEIP

SEQ ID NO: 123 - protein, 164 aa, Equus caballus
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFSCQDKQNTVCEECPDGTYSDEANHRDPCLPCTVCEDTERQLRECTRWADAECEEIP

SEQ ID NO: 124 - protein, 164 aa, Equus caballus
KEVCPTDLYTHSGECCKACNLGEGVAQPCGANQTVCEPCLDSVTFSDVVSATEPCKPCTECVGLQSMSAPCVEADDAVCRCAYGYYQDETTGRCEACQVCEAGSGLVFSCQDKQNTVCEECPDGTYSDEANHVLPCLPCTVCEDTERQLRECTRWADAECEEIP

SEQ ID NO: 125 - protein, 208 aa, Bos taurus
  REGION 165..192: note = stalk region
  REGION 193..208: note = alpha and gamma secretase sites
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRATPPEGSDSTDPSTQEPEVPPEQDLVTSTVSDVVTTVM

SEQ ID NO: 126 - DNA, 624 bp, Bos taurus
  source 1..624: mol_type = other DNA
atggggtcaggtgccgccggccgcgccatggacgggccgcgcctgctgctgctgctgctgctgctcctgggggtgtcccttggaggtgccaaggaagcatgcctcacgggcctgtacacccacagcggagagtgctgcaaagcctgcaacctgggcgagggtgtggcccagccttgtggagccaaccagaccgtgtgtgaaccctgcctggacagcgtgaccttctcggacgtggtgagcgccacggagccgtgtaagccgtgcacggagtgcgtgggactgcagagcatgtcggcgccctgcgtggaggccgacgacgccgtgtgccgctgcgcctacggctattaccaggacgagacgaccggccgctgcgaggcgtgccgcgtgtgcgaggcgggctcggggctcgtgttctcgtgccaggacaagcagaacaccgtctgcgaggagtgccccgacggcacgtactccgacgaggccaaccacgtggacccctgcctgccctgcacggtgtgcgaggacacggagcgccagctgcgcgagtgcacgcgctgggccgacgccgagtgcgaggagatccctggacgttggattacacgggccacgccccctgagggctccgac

SEQ ID NO: 127 - protein, 164 aa, Bos taurus
KEACLTGLYTHSGECCKACNLGEGVAQPCGANQTVCEPCLDSVTFSDVVSATEPCKPCTECVGLQSMSAPCVEATDAVCRCAYGYYQDETTGRCEACRVCEAGSGLVFSCQDKQNTVCEECPDGTYSDEANHVDPCLPCTVCEDTERQLRECTRWADAECEEIP

SEQ ID NO: 128 - protein, 164 aa, Bos taurus
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFYCQDKQNTVCEECPDGTYSDEANHVDPCLPCTVCEDTERQLRECTRWADAECEEIP

SEQ ID NO: 129 - protein, 164 aa, Bos taurus
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFHCQDKQNTVCEECPDGTYSDEANHVDPCLPCTVCEDTERQLRECTRWADAECEEIP

SEQ ID NO: 130 - protein, 164 aa, Bos taurus
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHRDPCLPCTVCEDTERQLRECTRWADAECEEIP

SEQ ID NO: 131 - protein, 164 aa, Bos taurus
KEACLTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVLPCLPCTVCEDTERQLRECTRWADAECEEIP